SEQUENCE LISTING (84 sequences)

SEQ ID NO: 1 - DNA, 482 bp, Artificial Sequence
  Humanized 2G1 VH-11801
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 2 - protein, 144 aa, Artificial Sequence
  Humanized 2G1 VH-11801 (amino acid)
MDWTWVFLFLLSVTAGVHSQVQLVQSGAEVKKPGASVKVSCKASGYTITDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 3 - protein, 10 aa, Artificial Sequence
  CDR1 Humanized 2G1 VH-11801 (amino acid)
GYTITDYNLD

SEQ ID NO: 4 - protein, 16 aa, Artificial Sequence
  CDR2 Humanized 2G1 VH-11801 (amino acid)
DISPNYGYTIYNQKFK

SEQ ID NO: 5 - protein, 16 aa, Artificial Sequence
  CDR3 Humanized 2G1 VH-11801 (amino acid)
RDIRYSGNSYKWYFDV

SEQ ID NO: 6 - DNA, 428 bp, Artificial Sequence
  Humanized 2G1 VL-1901
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 7 - protein, 127 aa, Artificial Sequence
  Humanized 2G1 VL-1901 (amino acid)
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 8 - protein, 11 aa, Artificial Sequence
  CDR1 Humanized 2G1 VL-1901 (amino acid)
RTSKSISKYLA

SEQ ID NO: 9 - protein, 7 aa, Artificial Sequence
  CDR2 Humanized 2G1 VL-1901 (amino acid)
SGSTLQS

SEQ ID NO: 10 - protein, 9 aa, Artificial Sequence
  CDR3 Humanized 2G1 VL-1901 (amino acid)
QQHNEYPYT

SEQ ID NO: 11 - protein, 9 aa, Artificial Sequence
  CDR3 Humanized 2G1 VL-1901 variant (Q->H mutation in CDR3) (amino acid)
HQHNEYPYT

SEQ ID NO: 12 - DNA, 428 bp, Artificial Sequence
  Humanized 2G1 VL-1901 variant (Q->H mutation in CDR3)
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcatcaacataatgaatacccgtacacgtttggccaggggaccaagctggagatcaaa

SEQ ID NO: 13 - protein, 127 aa, Artificial Sequence
  Humanized 2G1 VL-1901 variant (Q->H mutation in CDR3) (amino acid)
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCHQHNEYPYTFGQGTKLEIK

SEQ ID NO: 14 - protein, 11 aa, Artificial Sequence
  CDR1 Humanized 2G1 VL-1901 variant (T->H mutation in CDR1) (amino acid)
RHSKSISKYLA

SEQ ID NO: 15 - DNA, 428 bp, Artificial Sequence
  Humanized 2G1 VL-1901 variant (T->H mutation in CDR1)
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggcatagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 16 - protein, 127 aa, Artificial Sequence
  Humanized 2G1 VL-1901 variant (T->H mutation in CDR1) (amino acid)
MEAPAQLLFLLLLWLPDTTGDIQLTQSPSFLSASVGDRVTITCRHSKSISKYLAWYQQKPGKAPKLLIYSGSTLQSGVPSRFSGSGSGTEFTLTISSLQPEDFATYYCQQHNEYPYTFGQGTKLEIK

SEQ ID NO: 17 - protein, 11 aa, Artificial Sequence
  CDR1 Humanized 2G1 VH-11801 variant (I->H mutation in CDR1) (amino acid)
RHSKSISKYLA

SEQ ID NO: 18 - DNA, 482 bp, Artificial Sequence
  Humanized 2G1 VH-11801 variant (I->H mutation in CDR1)
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacacatacagactacaatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 19 - protein, 144 aa, Artificial Sequence
  Humanized 2G1 VH-11801 variant (I->H mutation in CDR1) (amino acid)
MDWTWVFLFLLSVTAGVHSQVQLVQSGAEVKKPGASVKVSCKASGYTHTDYNLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 20 - protein, 10 aa, Artificial Sequence
  CDR1 Humanized 2G1 VH-11801 variant (N->H mutation in CDR1) (amino acid)
GYTITDYHLD

SEQ ID NO: 21 - DNA, 481 bp, Artificial Sequence
  Humanized 2G1 VH-11801 variant (N->H mutation in CDR1)
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttggactgggtgcgacaggcccctggacaagggcttgagtggatgggagatattagtcctaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttc

SEQ ID NO: 22 - protein, 144 aa, Artificial Sequence
  Humanized 2G1 VH-11801 variant (N->H mutation in CDR1) (amino acid)
MDWTWVFLFLLSVTAGVHSQVQLVQSGAEVKKPGASVKVSCKASGYTITDYHLDWVRQAPGQGLEWMGDISPNYGYTIYNQKFKDRVTMTTDTSTSTAYMELRSLRSDDTAVYYCARRDIRYSGNSYKWYFDVWGQGTMVTVSS

SEQ ID NO: 23 - protein, 11 aa, Artificial Sequence
  CDR1 Humanized 2G1 VL-1901 variant (Y->H mutation in CDR1) (amino acid)
RTSKSISKHLA

SEQ ID NO: 24 - DNA, 428 bp, Artificial Sequence
  Humanized 2G1 VL-1901 variant (Y->H mutation in CDR1)
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaacatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaa

SEQ ID NO: 25 - protein, 127 aa, Artificial Sequence
  Humanized 2G1 VL-1901 variant (Y->H mutation in CDR1) (amino acid)
MEAPAQLLFLLLLWLPDTTGDIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIK

SEQ ID NO: 26 - protein, 17 aa, Artificial Sequence
  CDR2 Humanized 2G1 VH-11801 variant (Y->H mutation in CDR2)
DISPNHGYTIYNQKFKD

SEQ ID NO: 27 - DNA, 482 bp, Artificial Sequence
  Humanized 2G1 VH-11801 variant (Y->H mutation in CDR2)
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaaccatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 28 - protein, 144 aa, Artificial Sequence
  Humanized 2G1 VH-11801 variant (Y->H mutation in CDR2) (amino acid)
MDWTWVFLFLLSVTAGVHSQVQLVQSGAEVKKPGASVKVSCKASGYTITDYNLDWVRQAPGQGLEWMGDISPNHGYTIYNQKFKDRVTMTTDTSTSTAYMELRSLRSDDTAVYYCARRDIRYSGNSYKWYFDVWGQGTMVTVSS

SEQ ID NO: 29 - protein, 9 aa, Artificial Sequence
  CDR3 Humanized 2G1 VL-1901 variant (E->H mutation in CDR3) (amino acid)
QQHNHYPYT

SEQ ID NO: 30 - DNA, 428 bp, Artificial Sequence
  Humanized 2G1 VL-1901 variant (E->H mutation in CDR3)
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaatatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatcattacccgtacacgtttggccaggggaccaagctggagatcaaa

SEQ ID NO: 31 - protein, 127 aa, Artificial Sequence
  Humanized 2G1 VL-1901 variant (E->H mutation in CDR3) (amino acid)
MEAPAQLLFLLLLWLPDTTGDIQLTQSPSFLSASVGDRVTITCRTSKSISKYLAWYQQKPGKAPKLLIYSGSTLQSGVPSRFSGSGSGTEFTLTISSLQPEDFATYYCQQHNHYPYTFGQGTKLEIK

SEQ ID NO: 32 - protein, 327 aa, Artificial Sequence
  Human IgG4 Constant Heavy chain region (amino acid)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 327 aa, Artificial Sequence
  Human IgG4 Constant Heavy chain region with S228P mutation (amino acid)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 17 aa, Artificial Sequence
  CDR2 Humanized 2G1 VH-11801 variant (T->H mutation in CDR2)
DISPNYGYHIYNQKFKD

SEQ ID NO: 35 - DNA, 482 bp, Artificial Sequence
  Humanized 2G1 VH-11801 variant (T->H mutation in CDR2)
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactacaatttggactgggtgcgacaggcccctggacaagggcttgagtggatgggagatattagtcctaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgtctggggccaagggacaatggtcaccgtctcttca

SEQ ID NO: 36 - protein, 144 aa, Artificial Sequence
  Humanized 2G1 VH-11801 variant (T->H mutation in CDR2) (amino acid)
MDWTWVFLFLLSVTAGVHSQVQLVQSGAEVKKPGASVKVSCKASGYTITDYNLDWVRQAPGQGLEWMGDISPNYGYHIYNQKFKDRVTMTTDTSTSTAYMELRSLRSDDTAVYYCARRDIRYSGNSYKWYFDVWGQGTMVTVSS

SEQ ID NO: 37 - protein, 10 aa, Artificial Sequence
  CDR1 IWW-VH (amino acid)
GYTITDYHID

SEQ ID NO: 38 - protein, 17 aa, Artificial Sequence
  CDR2 IWW-VH (amino acid)
DISWNYGYTIYNQKFKD

SEQ ID NO: 39 - protein, 16 aa, Artificial Sequence
  CDR3 IWW-VH (amino acid)
RDIRYSGNSYKWYFDW

SEQ ID NO: 40 - DNA, 482 bp, Artificial Sequence
  IWW-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 41 - protein, 144 aa, Artificial Sequence
  IWW-VH (amino acid)
MDWTWVFLFLLSVTAGVHSQVQLVQSGAEVKKPGASVKVSCKASGYTITDYHIDWVRQAPGQGLEWMGDISWNYGYTIYNQKFKDRVTMTTDTSTSTAYMELRSLRSDDTAVYYCARRDIRYSGNSYKWYFDWWGQGTMVTVSS

SEQ ID NO: 42 - protein, 10 aa, Artificial Sequence
  CDR1 IFW-VH (amino acid)
GYTITDYHID

SEQ ID NO: 43 - protein, 17 aa, Artificial Sequence
  CDR2 IFW-VH (amino acid)
DISFNYGYTIYNQKFKD

SEQ ID NO: 44 - protein, 16 aa, Artificial Sequence
  CDR3 IFW-VH (amino acid)
RDIRYSGNSYKWYFDW

SEQ ID NO: 45 - DNA, 482 bp, Artificial Sequence
  IFW-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 46 - protein, 144 aa, Artificial Sequence
  IFW-VH (amino acid)
MDWTWVFLFLLSVTAGVHSQVQLVQSGAEVKKPGASVKVSCKASGYTITDYHIDWVRQAPGQGLEWMGDISFNYGYTIYNQKFKDRVTMTTDTSTSTAYMELRSLRSDDTAVYYCARRDIRYSGNSYKWYFDWWGQGTMVTVSS

SEQ ID NO: 47 - protein, 10 aa, Artificial Sequence
  CDR1 FME-VH (amino acid)
GYTITDYHFD

SEQ ID NO: 48 - protein, 17 aa, Artificial Sequence
  CDR2 FME-VH (amino acid)
DISMNYGYTIYNQKFKD

SEQ ID NO: 49 - protein, 16 aa, Artificial Sequence
  CDR3 FME-VH (amino acid)
RDIRYSGNSYKWYFDE

SEQ ID NO: 50 - DNA, 482 bp, Artificial Sequence
  FME-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttca

SEQ ID NO: 51 - protein, 144 aa, Artificial Sequence
  FME-VH (amino acid)
MDWTWVFLFLLSVTAGVHSQVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDEWGQGTMVTVSS

SEQ ID NO: 52 - protein, 10 aa, Artificial Sequence
  CDR1 FMW-VH (amino acid)
GYTITDYHFD

SEQ ID NO: 53 - protein, 17 aa, Artificial Sequence
  CDR2 FMW-VH (amino acid)
DISMNYGYTIYNQKFKD

SEQ ID NO: 54 - protein, 16 aa, Artificial Sequence
  CDR3 FMW-VH (amino acid)
RDIRYSGNSYKWYFDW

SEQ ID NO: 55 - DNA, 482 bp, Artificial Sequence
  FMW-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatactatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 56 - protein, 144 aa, Artificial Sequence
  FMW-VH (amino acid)
MDWTWVFLFLLSVTAGVHSQVQLVQSGAEVKKPGASVKVSCKASGYTITDYHFDWVRQAPGQGLEWMGDISMNYGYTIYNQKFKDRVTMTTDTSTSTAYMELRSLRSDDTAVYYCARRDIRYSGNSYKWYFDWWGQGTMVTVSS

SEQ ID NO: 57 - protein, 17 aa, Artificial Sequence
  CDR2 FMEH-VH (amino acid)
DISMNYGYHIYNQKFKD

SEQ ID NO: 58 - DNA, 482 bp, Artificial Sequence
  FMEH-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttca

SEQ ID NO: 59 - protein, 144 aa, Artificial Sequence
  FMEH-VH (amino acid)
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSS

SEQ ID NO: 60 - DNA, 984 bp, Artificial Sequence
  Human IgG4 Fc PLA domain mutation
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaatga

SEQ ID NO: 61 - protein, 327 aa, Artificial Sequence
  Human IgG4 Fc PLA domain mutation (amino acid)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 62 - protein, 17 aa, Artificial Sequence
  CDR2 IWWH-VH (amino acid)
DISWNYGYHIYNQKFKD

SEQ ID NO: 63 - DNA, 482 bp, Artificial Sequence
  IWWH-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 64 - protein, 144 aa, Artificial Sequence
  IWWH-VH (amino acid)
MDWTWVFLFLLSVTAGVHSQVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSS

SEQ ID NO: 65 - protein, 17 aa, Artificial Sequence
  CDR2 IFWH-VH (amino acid)
DISFNYGYHIYNQKFKD

SEQ ID NO: 66 - DNA, 482 bp, Artificial Sequence
  IFWH-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 67 - protein, 144 aa, Artificial Sequence
  IFWH-VH (amino acid)
MDWTWVFLFLLSVTAGVHSQVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSS

SEQ ID NO: 68 - protein, 17 aa, Artificial Sequence
  CDR2 FMWH-VH (amino acid)
DISMNYGYHIYNQKFKD

SEQ ID NO: 69 - DNA, 482 bp, Artificial Sequence
  FMWH-VH
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttca

SEQ ID NO: 70 - protein, 144 aa, Artificial Sequence
  FMWH-VH (amino acid)
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSS

SEQ ID NO: 71 - DNA, 2384 bp, Artificial Sequence
  VH sequence of FMEH-IgG4PLA-FH1-5
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgatgagtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 72 - protein, 777 aa, Artificial Sequence
  VH sequence of FMEH-IgG4PLA-FH1-5  (amino acid)
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDEWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 73 - DNA, 752 bp, Artificial Sequence
  VL sequence of FMEH-IgG4-FH1-5
gtcagagccctggggaggaactgctcagttaggacccagagggaaccatggaagccccagctcagcttctcttcctcctgctactctggctcccagataccaccggagacatccagttgacccagtctccatccttcctgtctgcatctgtaggagacagagtcaccatcacttgcaggacaagtaagagcataagcaaacatttagcctggtatcagcaaaaaccagggaaagcccctaagctcctgatctattctggatccaccttgcaatctggggtcccatcaaggttcagcggcagtggatctgggacagaattcactctcacaatcagcagcctgcagcctgaagattttgcaacttattactgtcaacaacataatgaatacccgtacacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 74 - protein, 234 aa, Artificial Sequence
  VL sequence of FMEH-IgG4-FH1-5 (amino acid)
MEAPAQLLFLLLLWLPDTTGDIQLTQSPSFLSASVGDRVTITCRTSKSISKHLAWYQQKPGKAPKLLIYSGSTLQSGVPSRFSGSGSGTEFTLTISSLQPEDFATYYCQQHNEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 2384 bp, Artificial Sequence
  VH sequence of IWWH-IgG4PLA-FH1-5
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagttggaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 76 - protein, 777 aa, Artificial Sequence
  VH sequence of IWWH-IgG4PLA-FH1-5 (amino acid)
MDWTWVFLFLLSVTAGVHSQVQLVQSGAEVKKPGASVKVSCKASGYTITDYHIDWVRQAPGQGLEWMGDISWNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 77 - DNA, 2384 bp, Artificial Sequence
  VH sequence of IFWH-IgG4PLA-FH1-5
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatatcgactgggtgcgacaggcccctggacaagggcttgagtggatgggagatattagtttcaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 78 - protein, 777 aa, Artificial Sequence
  VH sequence of IFWH-IgG4PLA-FH1-5 (amino acid)
MDWTWVFLFLLSVTAGVHSQVQLVQSGAEVKKPGASVKVSCKASGYTITDYHIDWVRQAPGQGLEWMGDISFNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 79 - DNA, 2384 bp, Artificial Sequence
  VH sequence of FMWH-IgG4PLA-FH1-5
cagcatatgatcagtgtcctctccaaagtccttgaacatagactctaaccatggactggacctgggtctttctcttcctcctgtcagtaactgcaggtgtccactcccaggttcagctggtgcagtctggagctgaggtgaagaagcctggggcctcagtgaaggtctcctgcaaggcttctggatacacaatcacagactaccatttcgactgggtgcgacaggcccctggacaagggcttgagtggatgggagatattagtatgaactatggttatcatatctacaaccagaaattcaaggacagagtcaccatgaccacagacacatccacgagcacagcctacatggagctgaggagcctgagatctgacgacacggccgtgtattactgtgcgagaagggacattcgttactccggtaattcctacaaatggtacttcgattggtggggccaagggacaatggtcaccgtctcttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 80 - protein, 777 aa, Artificial Sequence
  VH sequence of FMWH-IgG4PLA-FH1-5 (amino acid)
MDWTWVFLFLLSVTAGVHSQVQLVQSGAEVKKPGASVKVSCKASGYTITDYHFDWVRQAPGQGLEWMGDISMNYGYHIYNQKFKDRVTMTTDTSTSTAYMELRSLRSDDTAVYYCARRDIRYSGNSYKWYFDWWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 81 - protein, 1231 aa, Artificial Sequence
  Amino Acid sequence of P08603-1; Human Complement Factor H Isoform 1, precursor
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 82 - protein, 1213 aa, Artificial Sequence
  Amino Acid sequence of P08603-1; Human Complement Factor H Isoform 1
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 83 - protein, 449 aa, Artificial Sequence
  Amino Acid sequence of P08603-2; Human Complement Factor H Isoform 2, precursor
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 84 - protein, 431 aa, Artificial Sequence
  Amino Acid sequence of P08603-2; Human Complement Factor H Isoform 2
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

(19) United States
(12) Patent Application Publication
(10) Pub. No.: US 2022/0204602 A1
(43) Pub. Date: Jun. 30, 2022

(54) BI-FUNCTIONAL HUMANIZED ANTI-C5 ANTIBODIES AND FACTOR H FUSION PROTEINS AND USES THEREOF

(71) Applicants: The Trustees of the University of Pennsylvania, Philadelphia, PA (US); Kira Pharmaceuticals (US) LLC, Arlington, MA (US)

(72) Inventors: Wenchao Song, Bryn Mawr, PA (US); Takashi Miwa, Bala Cynwyd, PA (US); Damodar Gullipalli, Philadelphia, PA (US); Sayaka Sato, Philadelphia, PA (US); Ping Tsui, North Potomac, MD (US); Yingjie Zhu, Quaker Hill, CT (US); Xihua Zhu, Suzhou (CN)

(21) Appl. No.: 17/605,695

(22) PCT Filed: Apr. 24, 2020
(86) PCT No.: PCT/US2020/029876

§ 371 Date: Oct. 22, 2021

Related U.S. Application Data
(60) Provisional application No. 62/837,853, filed on Apr. 24, 2019.
(60) Provisional application No. 62/837,833, filed on Apr. 24, 2019.

(51) Int. Cl. C07K 16/18; C07K 14/47; A61P 37/02; A01K 67/027

(52) U.S. Cl. CPC C07K 16/18; C07K 14/472; A61P 37/02; A61K 38/00; C07K 2319/30; C07K 2317/76; C07K 2317/565; A01K 67/0276

(57) ABSTRACT

This invention relates to inhibition of the complement signaling using an anti-C5 antibody or fusion protein thereof. Specifically, the invention relates to methods of treating a complement-mediated disease or complement-mediated disorder in an individual by contacting the individual with an anti-C5 antibody fusion protein thereof.

### CROSS-REFERENCE TO RELATED APPLICATIONS

This application claims priority under 35 U.S.C. § 119(e) to U.S. Provisional Patent Application Ser. No. 62/837,853, filed Apr. 24, 2019, and U.S. Provisional Patent Application Ser. No. 62/837,833, filed Apr. 24, 2019, the disclosure of which are incorporated herein by reference in their entireties.

### STATEMENT REGARDING FEDERALLY SPONSORED RESEARCH OR DEVELOPMENT

This invention was made with government support under NIH grant numbers AI085596 and AI117410 awarded by the National Institutes of Health (NIH). The government has certain rights in the invention.

### BACKGROUND OF THE INVENTION

The complement system is part of innate immunity that plays a key role in host defense. Normally the activation of complement is carefully controlled so that it does not cause autologous injury to host tissues. However, under certain situations where the regulatory mechanism is either defective (e.g., mutations in complement regulator genes) or inadequate (e.g., when there is massive autoantibody- or infection-induced complement activation that overwhelms the capacity of the regulators), severe and life-threatening autologous tissue injury by unbridled complement system may occur. Many autoimmune and inflammatory diseases are now known to be mediated by inappropriate complement activation, and there is an intense effort in the field to understand the pathogenic mechanism of various complement-mediated diseases and to develop specific anti-complement inhibitors as drugs to treat these disorders. Activated complement also has the potential to cause significant tissue injury and destruction and dysregulated complement activity has been found to be associated with a number of rare and common diseases such as paroxysmal nocturnal hemoglobinuria (PNH), atypical hemolytic uremic syndrome (aHUS), rheumatoid arthritis, age-related macular degeneration etc. Thus, anti-complement therapy is a promising way of treating these human disorders.

Complement C5 is a critical protein in the terminal pathway of complement activation and is the precursor protein for generating the potent pro-inflammatory mediator C5a, as well as the cytolytic membrane attack complex (MAC). C3 activation also leads to the generation of C5-cleaving enzyme complexes and initiates the terminal complement activation pathway, culminating in the production of the potent pro-inflammatory mediator C5a and the membrane attack complex C5b-9 which can cause cell lysis and death.

A number of human inflammatory and autoimmune diseases are mediated by C5a and/or MAC, and blocking C5 activation should prevent C5a and MAC generation and be of therapeutic value. A humanized mouse anti-human C5 mAb, eculizumab, has been used to treat two complement-mediated diseases PNH and aHUS. However, not all PNH patients are responsive to eculizumab treatments and one of the reasons for non-responsiveness is genetic polymorphism of human C5 with loss of epitope binding to eculizumab. Additionally, due to high plasma concentration of C5 and targeted-mediated rapid removal of antibody, eculizumab has to be administered to patients at high doses and frequency.

One of the challenges in developing drugs targeting complement proteins are their high plasma concentration and/or fast turnover. For example, the plasma concentrations of human C3 and C5 are approximately 1 mg/mL and 80 ug/mL, respectively. This means that inhibitors for such proteins need to be administered at a high dose and/or frequently. Indeed, the anti-C5 mAb drug Eculizumab is required to be given every two weeks through intravenous injection in PNH and aHUS patients at a maintenance dose of 900 mg and 1200 mg, respectively. Although a longer lasting second generation anti-C5 mAb Ravulizumab has been developed to lessen the injection frequency to every 8 weeks, the maintenance dose of Ravulizumab has increased to 3300 mg per injection. Furthermore, neither Eculizumab nor Ravulizumab was able to normalize LDH and hemoglobin levels in approximately 50% treated PNH patients. In PNH patients on standard Eculizumab therapy, breakthrough lysis are frequently observed and 20-30% patients are still transfusion-dependent. These unmet medical needs in PNH patients are related to the fact that deficiency of DAF and CD59 on affected blood cells makes them susceptible to C3 activation as well as MAC-mediated injury. Although anti-C5 mAbs, such as Eculizumab and Ravulizumab, can inhibit C5-mediated hemolysis, they do not prevent C3 activation on affected RBCs and as a result, C3b opsonization of RBCs still happens and this leads to the well-recognized phenomenon of extravascular hemolysis (EVH), a process caused by phagocytosis of C3b-opsonized RBCs in the reticuloendothelial system. In addition, studies have shown that blocking complement activation on RBCs at the C5 step has its limitation in efficacy because if too many C5 convertases are already assembled on the cell surface, it becomes impossible to block C5 cleavage completely with mAbs of a finite avidity. This may explain the breakthrough lysis phenomenon in PNH patients treated with Eculizumab and why anti-C5 mAbs are not capable of preventing complete hemolysis of rabbit and PNH RBC cells in ex vivo assays, as both PNH and rabbit RBCs are exceedingly sensitive to C3 complement activation via the AP and can easily assemble abundant C5 convertases on the surface. Although there are ongoing efforts to target C3 activation in complement-dependent diseases, e.g., through the use of C3-inhibitory cyclic peptides or recombinant short variants of Factor H (FH), such molecules have very poor pharmacokinetics and require large and frequent (e.g., daily) dosing.

Thus, there is a need in the art for long-lasting and bi-functional complement inhibitors that can inhibit both C3 and C5 activities and thereby achieve higher potency in treating terminal complement-mediated pathologies as well as provide convenience of less frequent dosing. The present invention addresses and meets these and other needs.

### SUMMARY OF THE INVENTION

In one embodiment, the invention comprises a fusion protein comprising an antibody that specifically binds to human C5 and a fusion protein partner. In one embodiment, the C5 is human C5. In one embodiment, the antibody is a monoclonal antibody. In one embodiment, the antibody is a humanized antibody. In one embodiment, the antibody is a chimeric antibody. In some embodiments, the antibody is a full-length antibody. In some embodiments, the antibody is an antibody fragment, which includes, but is not limited to, Fab, Fab′, F(ab)2, F(ab′)2, and scFv. In some embodiments, the antibody is part of a construct, for example a fusion construct comprising the antibody and a targeting moiety or an effector moiety. In some embodiments, the antibody is part of a conjugate construct, such as an antibody drug conjugate construct.

In one aspect of the invention, the antibody fusion protein exhibits pH-dependent binding to C5. In some embodiments, the pH-dependent antibody fusion protein binds more strongly to C5 at a more neutral pH (e.g., about pH 7.4; such as that found in the blood) than it does at a more acidic pH (e.g., about pH 5.8; such as that found in the endosome). In some embodiments, the pH-dependent antibody fusion protein dissociates more quickly from C5 at a more acidic pH (e.g., about pH 5.8; such as that found in the endosome) than it does at neutral pH (e.g., about pH 7.4; such as that found in the blood).

In some embodiments, the anti-C5 antibody exhibits pH-dependent binding to C5. In some embodiments, the pH-dependent anti-C5 antibody binds more strongly to C5 at a more neutral pH (e.g., about pH 7.4; such as that found in the blood) than it does at a more acidic pH (e.g., about pH 5.8; such as that found in the endosome). In some embodiments, the pH-dependent anti-C5 antibody dissociate more quickly from C5 at a more acidic pH (e.g., about pH 5.8; such as that found in the endosome) than it does at neutral pH (e.g., about pH 7.4; such as that found in the blood).

In one embodiment, the fusion protein comprises a complement control protein or a fragment of a complement control protein. In one embodiment, the complement control protein or fragment of complement control protein is an inhibitor of C3 convertase. In one embodiment, the C3 convertase is the alternative pathway C3 convertase C3bBb. In one embodiment, the C3 convertase is the classical pathway C3 convertase C4b2a. In one embodiment, the complement control protein or fragment of complement control protein is an inhibitor of complement activation steps other than C3 or C5 activation. In various embodiments, the fusion protein partner comprises a complement receptor 1 (CR1) or a fragment thereof, a membrane cofactor protein (MCP) or a factor thereof, a C4b-binding protein (C4BP) or a fragment thereof, a decay-accelerating factor (DAF) or a fragment thereof, an Apolipoprotein E (ApoE) or a fragment thereof, a FH protein or a fragment thereof, a human IgG4 or a fragment thereof, a linker, or any combination thereof. In one embodiment, the fragment of FH comprises short consensus repeat (SCR) domains 1-5 of the FH protein. In one embodiment, the fragment of DAF is the extracellular domain of DAF. In one embodiment, the fragment of CR1 is selected SCRs of the extracellular domain of CR1.

In one embodiment, the fusion protein comprises a fusion protein partner bound to the antibody. In one embodiment, the fusion protein comprises a fusion protein partner bound to the antibody with at least one linker. In one embodiment, the fusion protein comprises a fusion protein partner bound to the antibody without a linker. In one embodiment, the fusion protein comprises a fusion protein partner bound to C-terminal of the antibody. In one embodiment, the fusion protein comprises a fusion protein partner bound to N-terminal of the antibody.

In one embodiment, the fusion protein comprises a fusion protein partner bound to the anti-C5 mAb. In one embodiment, the fusion protein comprises a fusion protein partner bound to the anti-C5 mAb with at least one linker. In one embodiment, the fusion protein comprises a fusion protein partner bound to the anti-C5 mAb without a linker. In one embodiment, the fusion protein comprises a fusion protein partner bound to C-terminal of the anti-C5 mAb. In one embodiment, the fusion protein comprises a fusion protein partner bound to N-terminal of the anti-C5 mAb.

In one embodiment, the fusion protein comprises a fusion protein partner bound to a VH sequence of the antibody. In one embodiment, the fusion protein comprises a fusion protein partner bound to a VH sequence of the antibody with at least one linker. In one embodiment, the fusion protein comprises a fusion protein partner bound to a VH sequence of the antibody without a linker. In one embodiment, the fusion protein comprises a fusion protein partner bound to C-terminal of a VH sequence of the antibody. In one embodiment, the fusion protein comprises a fusion protein partner bound to N-terminal of a VH sequence of the antibody.

In one embodiment, the fusion protein comprises a fusion protein partner bound to a VL sequence of the antibody. In one embodiment, the fusion protein comprises a fusion protein partner bound to a VL sequence of the antibody with at least one linker. In one embodiment, the fusion protein comprises a fusion protein partner bound to a VL sequence of the antibody without a linker. In one embodiment, the fusion protein comprises a fusion protein partner bound to C-terminal of a VL sequence of the antibody. In one embodiment, the fusion protein comprises a fusion protein partner bound to N-terminal of a VL sequence of the antibody.

In one embodiment, the antibody comprises at least one of the complementarity-determining regions (CDRs) selected from the group consisting of: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:11, or a variant or variants thereof. In one embodiment, the antibody comprises the CDRs: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:11, or a variant or variants thereof.

In one embodiment, the antibody comprises a heavy chain comprising the amino acid sequence of SEQ ID NO:2, or a variant thereof. In one embodiment, the antibody comprises a light chain comprising the amino acid sequence of SEQ ID NO:13, or a variant thereof. In one embodiment, the antibody comprises a heavy chain comprising the amino acid sequence of SEQ ID NO:2 and a light chain comprising the amino acid sequence of SEQ ID NO:13, or a variant or variants thereof.

In one embodiment, the antibody comprises at least one of the CDRs selected from the group consisting of: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof. In one embodiment, the antibody comprises the CDRs: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:2, or a variant thereof. In one embodiment, the antibody comprises a light chain comprising the amino acid sequence of SEQ ID NO:16, or a variant thereof. In one embodiment, the antibody comprises a heavy chain comprising the amino acid sequence of SEQ ID NO:2 and a light chain comprising the amino acid sequence of SEQ ID NO:16, or a variant or variants thereof.

In one embodiment, the antibody comprises at least one of the CDRs selected from the group consisting of: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In one embodiment, the antibody comprises the CDRs: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:19, or a variant thereof. In one embodiment, the antibody comprises a light chain comprising the amino acid sequence of SEQ ID NO:7, or a variant thereof. In one embodiment, the antibody comprises a heavy chain comprising the amino acid sequence of SEQ ID NO:19 and a light chain comprising the amino acid sequence of SEQ ID NO:7, or a variant or variants thereof.

In one embodiment, the antibody comprises at least one of the CDRs selected from the group consisting of: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:22, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:22 and a light chain comprising the amino acid sequence of SEQ ID NO:25, or a variant or variants thereof.

In one embodiment, the antibody comprises at least one of the CDRs selected from the group consisting of: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof. In one embodiment, the antibody comprises the CDRs: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof.

In one embodiment, the antibody comprises a heavy chain comprising the amino acid sequence of SEQ ID NO:28, or a variant thereof. In one embodiment, the antibody comprises a light chain comprising the amino acid sequence of SEQ ID NO:31, or a variant thereof. In one embodiment, the antibody comprises a heavy chain comprising the amino acid sequence of SEQ ID NO:28 and a light chain comprising the amino acid sequence of SEQ ID NO:31, or a variant or variants thereof.

In one embodiment, the antibody comprises at least one of the CDRs selected from the group consisting of: VH-CDR1: SEQ ID NO:3; VH-CDR2: SEQ ID NO:34; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In one embodiment, the antibody comprises the CDRs: VH-CDR1: SEQ ID NO:3; VH-CDR2: SEQ ID NO:34; VH-CDR3: SEQ ID NO:5; VL-CDR1: SEQ ID NO:8; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:36, or a variant thereof. In one embodiment, the antibody comprises a light chain comprising the amino acid sequence of SEQ ID NO:7, or a variant thereof. In one embodiment, the antibody comprises a heavy chain comprising the amino acid sequence of SEQ ID NO:36 and a light chain comprising the amino acid sequence of SEQ ID NO:7, or a variant or variants thereof.

In one embodiment, the antibody comprises at least one of the CDRs selected from the group consisting of: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:41, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:41 and a light chain comprising the amino acid sequence of SEQ ID NO:25, or a variant or variants thereof.

In one embodiment, the antibody comprises at least one of the CDRs selected from the group consisting of: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:46, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:46 and a light chain comprising the amino acid sequence of SEQ ID NO:25, or a variant or variants thereof.

In one embodiment, the antibody comprises at least one of the CDRs selected from the group consisting of: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:51, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:51 and a light chain comprising the amino acid sequence of SEQ ID NO:25, or a variant or variants thereof.

In one embodiment, the antibody comprises at least one of the CDRs selected from the group consisting of: VH-CDR1: SEQ ID NO:52; VH-CDR2: SEQ ID NO:53; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:52; VH-CDR2: SEQ ID NO:53; VH-CDR3: SEQ ID NO:54; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:56, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:56 and a light chain comprising the amino acid sequence of SEQ ID NO:25, or a variant or variants thereof.

In one embodiment, the antibody comprises at least one of the CDRs selected from the group consisting of: VH-CDR1: SEQ ID NO:47; VH-CDR2: SEQ ID NO:57; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:47; VH-CDR2: SEQ ID NO:57; VH-CDR3: SEQ ID NO:49; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:59, or a variant thereof. In one embodiment, the antibody comprises a light chain comprising the amino acid sequence of SEQ ID NO:25, or a variant thereof. In one embodiment, the antibody comprises a heavy chain comprising the amino acid sequence of SEQ ID NO:59 and a light chain comprising the amino acid sequence of SEQ ID NO:25, or a variant or variants thereof.

In one embodiment, the antibody comprises a heavy chain comprising the amino acid sequence of SEQ ID NO:72, or a variant thereof. In one embodiment, the antibody comprises a light chain comprising the amino acid sequence of SEQ ID NO:74, or a variant thereof. In one embodiment, the antibody comprises a heavy chain comprising the amino acid sequence of SEQ ID NO:72 and a light chain comprising the amino acid sequence of SEQ ID NO:74, or a variant or variants thereof.

In one embodiment, the antibody comprises at least one of the CDRs selected from the group consisting of: VH-CDR1: SEQ ID NO:37; VH-CDR2: SEQ ID NO:62; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:37; VH-CDR2: SEQ ID NO:62; VH-CDR3: SEQ ID NO:39; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:76, or a variant thereof. In one embodiment, the antibody comprises a light chain comprising the amino acid sequence of SEQ ID NO:74, or a variant thereof. In one embodiment, the antibody comprises a heavy chain comprising the amino acid sequence of SEQ ID NO:76 and a light chain comprising the amino acid sequence of SEQ ID NO:74, or a variant or variants thereof.

In one embodiment, the antibody comprises at least one of the CDRs selected from the group consisting of: VH-CDR1: SEQ ID NO:42; VH-CDR2: SEQ ID NO:65; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:42; VH-CDR2: SEQ ID NO:65; VH-CDR3: SEQ ID NO:44; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:78, or a variant thereof. In one embodiment, the antibody comprises a light chain comprising the amino acid sequence of SEQ ID NO:74, or a variant thereof. In one embodiment, the antibody comprises a heavy chain comprising the amino acid sequence of SEQ ID NO:78 and a light chain comprising the amino acid sequence of SEQ ID NO:74, or a variant or variants thereof.

In one embodiment, the antibody comprises at least one of the CDRs selected from the group consisting of: VH-CDR1: SEQ ID NO:52; VH-CDR2: SEQ ID NO:68; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In one embodiment, the antibody comprises the CDRs: VH-CDR1: SEQ ID NO:52; VH-CDR2: SEQ ID NO:68; VH-CDR3: SEQ ID NO:54; VL-CDR1: SEQ ID NO:23; VL-CDR2: SEQ ID NO:9; and VL-CDR3: SEQ ID NO:10, or a variant or variants thereof.

In one embodiment, the antibody comprises a heavy chain comprising the amino acid sequence of SEQ ID NO:80, or a variant thereof. In one embodiment, the antibody comprises a light chain comprising the amino acid sequence of SEQ ID NO:74, or a variant thereof. In one embodiment, the antibody comprises a heavy chain comprising the amino acid sequence of SEQ ID NO:80 and a light chain comprising the amino acid sequence of SEQ ID NO:74, or a variant or variants thereof.

In one embodiment, the antibody is at least one selected from the group consisting of mAbs L3-1, L1-2, H1-4, H1-8/L1-9, and H2-6/L3-5. In one embodiment, antibody is a mAb H1-8/L1-9 variation.

In some embodiments, the anti-C5 antibody, fusion protein, or antigen-binding fragment thereof comprises a substitution of the proline residue at position #4 (i.e., P4) in VH CDR2, relative to SEQ ID NO:4. In various embodiments, the substitution at P4 is P4→F4 (i.e., P4F), P4→L4 (i.e., P4L), P4→M4 (i.e., P4M), P4→W4 (i.e., P4W), or P4→I4 (i.e., P4I).

In some embodiments, the anti-C5 antibody, fusion protein, or antigen-binding fragment thereof comprises a substitution of the threonine residue at position #9 (i.e., T9) in VH CDR2, relative to SEQ ID NO:4. In various embodiments, the substitution at T9 is T9→H9 (i.e., T9H), T9→F9 (i.e., T9F), T9→L9 (i.e., T9L), T9→M9 (i.e., T9M), T9→W9 (i.e., T9W), or T9→I9 (i.e., T9I). In some embodiments, the anti-C5 antibody, fusion protein, or antigen-binding fragment thereof comprises a substitution of the proline residue at position #4 (i.e., P4) in VH CDR2, relative to SEQ ID NO:4, and a substitution of the threonine residue at position #9 (i.e., T9) in VH CDR2, relative to SEQ ID NO:4. In various embodiments, the substitution at P4 is P4→F4 (i.e., P4F), P4→L4 (i.e., P4L), P4→M4 (i.e., P4M), P4→W4 (i.e., P4W), or P4→I4 (i.e., P4I); and the substitution at T9 is T9→H9 (i.e., T9H), T9→F9 (i.e., T9F), T9→L9 (i.e., T9L), T9→M9 (i.e., T9M), T9→W9 (i.e., T9W), or T9→I9 (i.e., T9I).

In some embodiments, the anti-C5 antibody, fusion protein, or antigen-binding fragment thereof comprises a substitution of the valine residue at position #16 (i.e., V16) in VH CDR3, relative to SEQ ID NO:5. In various embodiments, the substitution at V16 is V16→F16 (i.e., V16F), V16→E16 (i.e., V16E) or V16→W16 (i.e., V16W).

In some embodiments, the anti-C5 antibody, fusion protein, or antigen-binding fragment thereof comprises a substitution of the leucine residue at position #9 (i.e., L9) in VH CDR1, relative to SEQ ID NO:20. In various embodiments, the substitution at L9 is L9→W9 (i.e., L9W), L9→I9 (i.e., L9I), L9→V9 (i.e., L9V), L9→Y9 (i.e., L9Y), or L9→F9 (i.e., L9F).

In some embodiments, the anti-C5 antibody, fusion protein, or antigen-binding fragment thereof comprises a substitution at two or more of the group consisting of proline 4 (i.e., P4) in VH CDR2, relative to SEQ ID NO:4, threonine 9 (i.e., T9) in VH CDR2, relative to SEQ ID NO:4, valine 16 (i.e., V16) in VH CDR3, relative to SEQ ID NO:5, and leucine 9 (i.e., L9) in VH CDR1, relative to SEQ ID NO:20. In various embodiments, the anti-C5 antibody or antigen-binding fragment thereof comprising a substitution at two or more of the group consisting of proline 4 (i.e., P4) in VH CDR2, relative to SEQ ID NO:4, valine 16 (i.e., V16) in VH CDR3, relative to SEQ ID NO:5, and leucine 9 (i.e., L9) in VH CDR1, relative to SEQ ID NO:20 comprises the two or more substitutions selected from the group consisting of L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4W/V16E, L9I/P4F/V16E, L9F/P4M/V16E, L9F/P4W/V16E, L9F/P4F/V16E, L9I/P4M/T9H/V16W, L9I/P4W/T9H/V16W, L9I/P4F/T9H/V16W, L9F/P4M/T9H/V16W, L9F/P4W/T9H/V16W, L9F/P4F/T9H/V16W, L9I/P4M/T9H/V16E, L9I/P4W/T9H/V16E, L9I/P4F/T9H/V16E, L9F/P4M/T9H/V16E, L9F/P4W/T9H/V16E, and L9F/P4F/T9H/V16E.

In one embodiment, the present invention relates to a method of treating a complement pathway-mediated disease or disorder in an individual, comprising the step of administering to said individual the anti-C5 antibody or fusion protein. In one embodiment, the disease or disorder is at least selected from the group consisting of: macular degeneration (MD), age-related macular degeneration (AMD), ischemia reperfusion injury, arthritis, rheumatoid arthritis, asthma, allergic asthma, lupus, ulcerative colitis, stroke, post-surgery systemic inflammatory syndrome, chronic obstructive pulmonary disease (COPD), PNH syndrome, myasthenia gravis, neuromyelitis optica, (NMO), multiple sclerosis, delayed graft function, antibody-mediated rejection, aHUS, central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, or any combinations thereof. In some embodiments, the AP-mediated disease is C3 glomerulopathy. In some embodiments, the AP-mediated disease is macular degeneration, such as age-related macular degeneration. In one embodiment, administration of the anti-C5 antibody or fusion protein inhibits the generation of a C3a or C3b protein. In one embodiment, administration of the fusion protein inhibits the generation of a C5a or C5b protein. In one embodiment, administration of the anti-C5 antibody or fusion protein inhibits the generation of a C3a or C3b protein, inhibits the generation of a C5a or C5b protein, or any combination thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:11, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:52; VH-CDR2: SEQ ID NO:53; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering a fusion protein to the individual via a route of administration selected from the group consisting of enteral administration, parenteral administration, and a combination thereof, and wherein the fusion protein comprises six complementarity determining regions having the following amino acid sequences: VH-CDR1: SEQ ID NO:47; VH-CDR2: SEQ ID NO:57; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In one embodiment, the fusion protein comprises an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering an antibody, or a fusion protein or fragment thereof, to the individual via a route of administration selected from the group consisting of enteral administration, parenteral administration, and a combination thereof, and wherein the antibody comprises six complementarity determining regions having the following amino acid sequences: VH-CDR1: SEQ ID NO:37; VH-CDR2: SEQ ID NO:62; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In one embodiment, the antibody is an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering an antibody, or a fusion protein or fragment thereof, to the individual via a route of administration selected from the group consisting of enteral administration, parenteral administration, and a combination thereof, and wherein the antibody comprises six complementarity determining regions having the following amino acid sequences: VH-CDR1: SEQ ID NO:42; VH-CDR2: SEQ ID NO:65; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In one embodiment, the antibody is an antibody fragment selected from the group consisting of a Fab, Fab′, F(ab)2, F(ab′)2, scFv, and combinations thereof.

In one embodiment, the present invention relates to a method of reducing the activity of a complement system of an individual, wherein the method comprises administering an antibody, or a fusion protein or fragment thereof, to the individual via a route of administration selected from the group consisting of enteral administration, parenteral administration, and a combination thereof, and wherein the antibody comprises six complementarity determining regions having the following amino acid sequences: VH-CDR1: SEQ ID NO:52; VH-CDR2: SEQ ID NO:68; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, wherein the antibody has a heavy chain variable (VH) region that has an amino acid sequence that is more than about 90% (such as more than any of 91%, 92%, 93%, 94%, 95%, 96%, 97%, 98%, or 99%) identical to SEQ ID NO:2, or a variant thereof. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, wherein the antibody has a light chain variable (VL) region that has an amino acid sequence that is more than about 90% (such as more than any of 91%, 92%, 93%, 94%, 95%, 96%, 97%, 98%, or 99%) identical to SEQ ID NO:7, or a variant thereof. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:2, and wherein the VL region has an amino acid sequence that is more than about 90% (such as more than any of 91%, 92%, 93%, 94%, 95%, 96%, 97%, 98%, or 99%) identical to SEQ ID NO:7. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, wherein the antibody has a VL region that has an amino acid sequence that is more than about 90% (such as more than any of 91%, 92%, 93%, 94%, 95%, 96%, 97%, 98%, or 99%) identical to SEQ ID NO:13, or a variant thereof. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:2, and wherein the VL region has an amino acid sequence that is more than about 90% (such as more than any of 91%, 92%, 93%, 94%, 95%, 96%, 97%, 98%, or 99%) identical to SEQ ID NO:13. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VL region that has an amino acid sequence that is more than about 90% (such as more than any of 91%, 92%, 93%, 94%, 95%, 96%, 97%, 98%, or 99%) identical to SEQ ID NO:16. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:2, and wherein the VL region has an amino acid sequence that is more than about 90% (such as more than any of 91%, 92%, 93%, 94%, 95%, 96%, 97%, 98%, or 99%) identical to SEQ ID NO:16. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:19. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VL region that has an amino acid sequence that is more than about 90% (such as more than any of 91%, 92%, 93%, 94%, 95%, 96%, 97%, 98%, or 99%) identical to SEQ ID NO:7. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:19, and wherein the VL region has an amino acid sequence that is more than about 90% (such as more than any of 91%, 92%, 93%, 94%, 95%, 96%, 97%, 98%, or 99%) identical to SEQ ID NO:7. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:22. In one embodiment, the antibody is an antibody fragment selected from the group consisting of a Fab, Fab′, F(ab)2, F(ab′)2, scFv, and combinations thereof.

In one embodiment, the present invention relates to a fusion protein comprising an antibody against human C5, wherein the antibody has a VL region that has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:22, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:28. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VL region that has an amino acid sequence that is more than about 90% (such as more than any of 91%, 92%, 93%, 94%, 95%, 96%, 97%, 98%, or 99%) identical to SEQ ID NO:31. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:28, and wherein the VL region has an amino acid sequence that is more than about 90% (such as more than any of 91%, 92%, 93%, 94%, 95%, 96%, 97%, 98%, or 99%) identical to SEQ ID NO:31. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:41. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:41, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:46. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:46, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:51. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:51, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:56. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:56, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region that has an amino acid sequence that is more than about 90% (such as more than any of 91%, 92%, 93%, 94%, 95%, 96%, 97%, 98%, or 99%) identical to SEQ ID NO:59. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, wherein the antibody has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:59, and wherein the VL region has an amino acid sequence that is more than about 90% (such as more than any of 91%, 92%, 93%, 94%, 95%, 96%, 97%, 98%, or 99%) identical to SEQ ID NO:25. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, an antibody fusion protein with inhibitory activity against human C3, or a combination thereof, wherein the antibody or the antibody fusion protein has a VH region that has an amino acid sequence that is more than about 90% (such as more than any of 91%, 92%, 93%, 94%, 95%, 96%, 97%, 98%, or 99%) identical to SEQ ID NO:72, or a variant thereof. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, an antibody fusion protein with inhibitory activity against human C3, or a combination thereof, wherein the antibody or the antibody fusion protein has a VL region that has an amino acid sequence that is more than about 90% (such as more than any of 91%, 92%, 93%, 94%, 95%, 96%, 97%, 98%, or 99%) identical to SEQ ID NO:74, or a variant thereof. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, an antibody fusion protein with inhibitory activity against human C3, or a combination thereof, wherein the antibody or the antibody fusion protein has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:72, and wherein the VL region has an amino acid sequence that is more than about 90% (such as more than any of 91%, 92%, 93%, 94%, 95%, 96%, 97%, 98%, or 99%) identical to SEQ ID NO:74. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, an antibody fusion protein with inhibitory activity against human C3, or a combination thereof, wherein the antibody or the antibody fusion protein has a VH region that has an amino acid sequence that is more than about 90% (such as more than any of 91%, 92%, 93%, 94%, 95%, 96%, 97%, 98%, or 99%) identical to SEQ ID NO:76, or a variant thereof. In one embodiment, the antibody is an antibody fragment selected from the group consisting of a Fab, Fab′, F(ab)2, F(ab′)2, scFv, and combinations thereof.

In some embodiments, the present invention relates a fusion protein comprising an antibody against human C5, an antibody fusion protein with inhibitory activity against human C3, or a combination thereof, wherein the antibody or the antibody fusion protein has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:76, and wherein the VL region has an amino acid sequence that is more than about 90% (such as more than any of 91%, 92%, 93%, 94%, 95%, 96%, 97%, 98%, or 99%) identical to SEQ ID NO:74. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, an antibody fusion protein with inhibitory activity against human C3, or a combination thereof, wherein the antibody or the antibody fusion protein has a VH region that has an amino acid sequence that is more than about 90% (such as more than any of 91%, 92%, 93%, 94%, 95%, 96%, 97%, 98%, or 99%) identical to SEQ ID NO:78, or a variant thereof. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, an antibody fusion protein with inhibitory activity against human C3, or a combination thereof, wherein the antibody or the antibody fusion protein has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:78, and wherein the VL region has an amino acid sequence that is more than about 90% (such as more than any of 91%, 92%, 93%, 94%, 95%, 96%, 97%, 98%, or 99%) identical to SEQ ID NO:74. In one embodiment, the antibody is an antibody fragment selected from the group consisting of a Fab, Fab′, F(ab)2, F(ab′)2, scFv, and combinations thereof.

In some embodiments, the present invention is a fusion protein comprising an antibody against human C5, an antibody fusion protein with inhibitory activity against human C3, or a combination thereof, wherein the antibody or the antibody fusion protein has a VH region that has an amino acid sequence that is more than about 90% (such as more than any of 91%, 92%, 93%, 94%, 95%, 96%, 97%, 98%, or 99%) identical to SEQ ID NO:80, or a variant thereof. In one embodiment, the antibody is an antibody fragment selected from the group consisting of a Fab, Fab′, F(ab)2, F(ab′)2, scFv, and combinations thereof.

In some embodiments, the present invention relates to a fusion protein comprising an antibody against human C5, an antibody fusion protein with inhibitory activity against human C3, or a combination thereof, wherein the antibody or the antibody fusion protein has a VH region and a VL region, wherein the VH region has an amino acid sequence that is more than about 90% (such as more than any of 91%, 92%, 93%, 94%, 95%, 96%, 97%, 98%, or 99%) identical to SEQ ID NO:80, and wherein the VL region has an amino acid sequence that is more than about 90% (such as more than any of 91%, 92%, 93%, 94%, 95%, 96%, 97%, 98%, or 99%) identical to SEQ ID NO:74. In one embodiment, the antibody is an antibody fragment selected from the group consisting of a Fab, Fab′, F(ab)2, F(ab′)2, scFv, and combinations thereof.

In one embodiment, the present invention relates to a cell comprising at least one of the fusion proteins described elsewhere herein. In some embodiments, the cell produces at least one of the fusion proteins described elsewhere herein. In one embodiment, the cell is a hybridoma.

In one embodiment, the present invention is a cell line comprising at least one of the fusion proteins described elsewhere herein. In some embodiments, the cell line produces at least one of the fusion proteins described elsewhere herein. In some embodiments, the cell line is a hybridoma cell line.

In one embodiment, the present invention relates to a genetically modified non-human animal. In one embodiment, the genetically modified non-human animal expresses human C5. In one embodiment, the genetically modified non-human animal is a rodent. In one embodiment, the genetically modified non-human animal is a mouse. In one embodiment, the genetically modified non-human animal is a NOD/SCID mouse. In one embodiment, the genetically modified non-human animal is a FcRn/SCID mouse.

In one embodiment, the present invention relates to a fusion protein comprising an anti-C5 antibody moiety and an FH or a functional fragment thereof. In some embodiments, the anti-C5 moiety comprises at least one histidine substitution. In one embodiment, the anti-C5 moiety comprises an IgG4 chain. In one embodiment, the IgG4 chain comprises a PLA mutation. In one embodiment, the FH or functional fragment thereof comprises domains 1-5 of the FH protein.

### BRIEF DESCRIPTION OF THE DRAWINGS

The foregoing summary, as well as the following detailed description of exemplary embodiments of the invention, will be better understood when read in conjunction with the appended drawings. It should be understood, however, that the invention is not limited to the precise arrangements and instrumentalities of the embodiments shown in the drawings. In the drawings:

FIG. 1 depicts nucleotide and amino acid sequences of a humanized variable heavy chain (VH) of mAb 2G1 (humanized 2G1 VH-11801) and humanized variable light chain (VL) of mAb 2G1 (humanized 2G1 VL-1901). Humanization was achieved by CDR grating from murine mAb 2G1 VH into a germline encoded human VH frame (11801) and CDR grating from murine mAb 2G1 VL into a germline encoded human VL frame (1901). The amino acid sequences of signal peptides are underlined and that of CDR1, CDR2 and CDR3 is bolded and shaded.

FIG. 2 depicts nucleotide and amino acid sequences of mAb L3-1, humanized VH-11801 and humanized VL-1901 with a Q→H substitution in VL-CDR3.

FIG. 3 depicts nucleotide and amino acid sequences of mAb L1-2, humanized VH-11801 and humanized VL-1901 with a T→H substitution in VL-CDR1.

FIG. 4 depicts nucleotide and amino acid sequences of mAb H1-4, humanized VH-11801 and humanized VL-1901 with a I→H substitution in VH-CDR1.

FIG. 5 depicts nucleotide and amino acid sequences of mAb H1-8/L1-9, humanized VH-11801 and humanized VL-1901 with a N→H substitution in VH-CDR1 and a Y→H substitution in VL-CDR1.

FIG. 6 depicts nucleotide and amino acid sequences of mAb H2-6/L3-5, humanized VH-11801 and humanized VL-1901 with a Y→H substitution in VH-CDR2 and a E→H substitution in VL-CDR3.

FIG. 7 depicts an Octet tracing of C5 binding and dissociation of the parental humanized mAb 11801 (VH-11801 (SEQ ID NO:2) and VL-1901 (SEQ ID NO:7)) at pH 5.8 and pH 7.4.

FIG. 8 depicts an Octet tracing of C5 binding and dissociation of the mAb L3-1 at pH 5.8 and pH 7.4.

FIG. 9 depicts an Octet tracing of C5 binding and dissociation of the mAb L1-2 at pH 5.8 and pH 7.4.

FIG. 10 depicts an Octet tracing of C5 binding and dissociation of the mAb H1-4 at pH 5.8 and pH 7.4.

FIG. 11 depicts an Octet tracing of C5 binding and dissociation of the mAb H2-6/L3-5 at pH 5.8 and pH 7.4.

FIG. 12 depicts an Octet tracing of C5 binding and dissociation of the mAb H1-8/L1-9 at pH 5.8 and pH 7.4.

FIG. 13 depicts the results of a classical pathway complement-mediated sheep red blood cell lysis assay to assess the C5 inhibitory effect of the parental humanized mAb 11801 (VH-11801 (SEQ ID NO:2) and VL-1901 (SEQ ID NO:7)), and its variants mAb L1-2, mAb L3-1, and mAb H2-6/L3-5.

FIG. 14 depicts the results of a classical pathway complement-mediated sheep red blood cell lysis assay to assess the C5 inhibitory effect of the parental humanized mAb 11801 (VH-11801 (SEQ ID NO:2) and VL-1901 (SEQ ID NO:7)) and variant mAb H1-8/L1-9.

FIG. 15 depicts the results of a classical pathway complement-mediated sheep red blood cell lysis assay to assess the C5 inhibitory effect of the parental humanized mAb 11801 (VH-11801 (SEQ ID NO:2) and VL-1901 (SEQ ID NO:7)) and variant mAb H1-4 and mAb L3-1.

FIG. 16 depicts the results of an ELISA assay assessing the level of human C5 in the plasma of NOD/SCID mice genetically modified to express human C5. Ml, M3, M4 and M5 designate 4 representative mice.

FIG. 17 depicts the results of an assay assessing the level of human IgG4 in the plasma of NOD/SCID mice genetically modified to express human C5 after injection with recombinant chimeric human IgG4 mAbs H1-4, H1-8/L1-9, H2-6/L3-5, L3-1 or L1-2.

FIG. 18 depicts the results of a classical pathway complement-mediated chicken red blood cell assay assessing the pharmacodynamics of the parental humanized mAb 2G1 (VH-11801 (SEQ ID NO:2) and VL-1901 (SEQ ID NO:7)) in two NOD/SCID mice (Mo-03 and Mo-05) genetically modified to express human C5.

FIG. 19 depicts the results of a classical pathway complement-mediated chicken red blood cell assay assessing the pharmacodynamics of recombinant chimeric human IgG4 mAbs L3-1, L1-2, H1-4, H1-8/L1-9 and H2-6/L3-5 in NOD/SCID mice genetically modified to express human C5.

FIG. 20 depicts the results of ELISA assay demonstrating improved binding at pH 7.4 to C5 of mAb H1-8/L1-9 ScFV variants having at least one substitution at leucine 9 (i.e., L9) in VH CDR1, relative to SEQ ID NO:20, proline 4 (i.e., P4) in VH CDR2, relative to SEQ ID NO:4, and/or valine 16 (i.e., V16) in VH CDR3, relative to SEQ ID NO:5 (i.e., L9→W9 (i.e., L9W), L9→I9 (i.e., L9I), L9→V9 (i.e., L9V), L9→Y9 (i.e., L9Y), L9→F9 (i.e., L9F), P4→F4 (i.e., P4F), P4→L4 (i.e., P4L), P4→M4 (i.e., P4M), P4→W4 (i.e., P4W), P4→I4 (i.e., P4I), V16→F16 (i.e., V16F), V16→E16 (i.e., V16E) and V16→W16 (i.e., V16W)). Binding of mAb H1-8/L1-9 ScFV variants is shown in column 3 (OD450) and 4 (OD450 confirm) and that of the parental mAb H1-8/L1-9 ScFV is shown in column 8 (WT/OD450).

FIG. 21 depicts the results of an Octet assay assessing the relative C5 binding affinity of mAb H1-8/L1-9 variants expressed in Expi-CHO cells as human IgG4. Expi-CHO cells were transfected with H1-8 VH variants as specified and L1-9 VL (SEQ ID No: 23) and cell culture supernatant was assessed 2 days after transfection. For a given cell culture supernatant, the ratio of C5 binding response to that of antibody binding response was calculated and used as a measure of C5 binding affinity. Shown in the figure are calculated ratios from two separate Octet assays of a transfection experiment with mAb H1-8/L1-9 IgG4 variants having at least one substitution at leucine 9 (i.e., L9) in VH CDR1, relative to SEQ ID NO:20, proline 4 (i.e., P4) in VH CDR2, relative to SEQ ID NO:4, and/or valine 16 (i.e., V16) in VH CDR3, relative to SEQ ID NO:5 (i.e., L9→W9 (i.e., L9W), L9→I9 (i.e., L9I), L9→V9 (i.e., L9V), L9→Y9 (i.e., L9Y), L9→F9 (i.e., L9F), P4→F4 (i.e., P4F), P4→L4 (i.e., P4L), P4→M4 (i.e., P4M), P4→W4 (i.e., P4W), P4→I4 (i.e., P4I), V16→F16 (i.e., V16F), V16→E16 (i.e., V16E) and V16→W16 (i.e., V16W)).

FIG. 22 depicts the results of an Octet assay assessing the dissociation rate at pH 7.4 and pH 5.8, respectively, of C5 and mAb H1-8/L1-9 variants. The % decrease for each mAb at pH 7.4 and pH 5.8 from peak C5 binding response after switching from association phase to dissociation phase was calculated. Shown in the figure are calculated % decreases in two separate Octet assays of a transfection experiment with mAb H1-8/L1-9 IgG4 variants having at least one substitution at leucine 9 (i.e., L9) in VH CDR1, relative to SEQ ID NO:20, proline 4 (i.e., P4) in VH CDR2, relative to SEQ ID NO:4, and/or valine 16 (i.e., V16) in VH CDR3, relative to SEQ ID NO:5 (i.e., L9→W9 (i.e., L9W), L9→I9 (i.e., L9I), L9→V9 (i.e., L9V), L9→Y9 (i.e., L9Y), L9→F9 (i.e., L9F), P4→F4 (i.e., P4F), P4→L4 (i.e., P4L), P4→M4 (i.e., P4M), P4→W4 (i.e., P4W), P4→I4 (i.e., P4I), V16→F16 (i.e., V16F), V16→E16 (i.e., V16E) and V16→W16 (i.e., V16W)).

FIG. 23 lists 18 combination substitution variants (i.e., L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4W/V16E, L9I/P4F/V16E, L9F/P4M/V16E, L9F/P4W/V16E, and L9F/P4F/V16E). These combination variants were derived from 7 single variants of mAb H1-8/L1-9 IgG4 (i.e., L9I, L9F, P4M, P4W, P4F, V16E, V16W) that showed improved C5 binding affinity over the parental H1-8/L1-9 mAb and at the same time maintained differential pH 7.4 and pH 5.8 dissociation rate (FIG. 21 and FIG. 22).

FIG. 24 depicts the results of an Octet assay assessing the relative C5 binding affinity of mAb H1-8/L1-9 combination substitution variants expressed in Expi-CHO cells as human IgG4. Expi-CHO cells were transfected with H1-8 VH combination substitution variants and cell culture supernatant was assessed 2 days after transfection. For a given cell culture supernatant, the ratio of C5 binding response to that of antibody binding response was calculated and used as a measure of C5 binding affinity. Shown in the figure are calculated ratios from a transfection experiment with mAb H1-8/L1-9 combination substitution variants L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4W/V16E, L9I/P4F/V16E, L9F/P4M/V16E, L9F/P4W/V16E, and L9F/P4F/V16E.

FIG. 25 depicts the results of an Octet assay assessing the dissociation rate at pH 7.4 and pH 5.8, respectively, of C5 and mAb H1-8/L1-9 combination substitution variants. The % decrease for each mAb at pH 7.4 and pH 5.8 from peak C5 binding response after switching from association phase to dissociation phase was calculated. Shown in the figure are calculated % decreases in a transfection experiment with mAb H1-8/L1-9 IgG4 combination substitution variants L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4F/V16E, L9I/P4F/V16E, L9F/P4M/V16E, L9F/P4W/V16E, and L9F/P4F/V16E.

FIG. 26 depicts the results of experiments assessing the affinity of C5-binding at pH 7.4 of additional scFV mutants from mAb 1819 affinity maturation experiment, including clone 14C6 which corresponds to T to H mutation in position 9 of VH CDR2.

FIG. 27 depicts the results of experiments assessing the differential affinity of C5-binding at pH 7.4 and pH 5.8 of additional scFV mutants from mAb 1819 affinity maturation experiment, including clone 14C6 which corresponds to T to H mutation in position 9 of VH CDR2.

FIG. 28 depicts the results of experiments ranking additional scFV mutants from mAb 1819 affinity maturation experiment according to differential binding at pH 7.4 and pH 5.8, showing clone 14C6 being the top ranked mutant among this group of mutants. Clone 14C6 corresponds to T to H mutation in position 9 of VH CDR2.

FIG. 29 depicts nucleotide and amino acid sequences of mAb H1-8/L1-9 variant IWW-VH (top), humanized VH-11801 with N→H and L→I substitutions in VH-CDR1, a P→W substitution in VH-CDR2, and a V→W substitution in VH-CDR3; as well as nucleotide and amino acid sequences of mAb H1-8/L1-9 variant IFW-VH (bottom), humanized VH-11801 with N→H and L→I substitutions in VH-CDR1, a P→F substitution in VH-CDR2, and a V→W substitution in VH-CDR3.

FIG. 30 depicts nucleotide and amino acid sequences of mAb H1-8/L1-9 variant FME-VH (top), humanized VH-11801 with N→H and L→F substitutions in VH-CDR1, a P→M substitution in VH-CDR2, and a V→E substitution in VH-CDR3; as well as nucleotide and amino acid sequences of mAb H1-8/L1-9 variant FMW-VH (bottom), humanized VH-11801 with N→H and L→F substitutions in VH-CDR1, a P→M substitution in VH-CDR2, and a V→W substitution in VH-CDR3.

FIG. 31 depicts nucleotide and amino acid sequences of mAb H1-8/L1-9 variant FMEH-VH, humanized VH-11801 with N→H and L→F substitutions in VH-CDR1, P→M and T→H substitutions in VH-CDR2, and a V→E substitution in VH-CDR3

FIG. 32 depicts an Octet tracing of C5 binding and dissociation of the recombinant chimeric human IgG4 mAb H1-8/L1-9, FMW, IFW, FME, and IWW at pH 5.8 and pH 7.4.

FIG. 33 depicts the % of C5 binding dissociated from peak value for the recombinant chimeric human IgG4 mAb 11801 (VH-11801 (SEQ ID NO: 2) and VL-1901 (SEQ ID NO: 7), H1-8/L1-9, FMW, IFW, FME, and IWW at pH 7.4 and pH 5.8.

FIG. 34 depicts the results of a classical pathway complement-mediated sheep red blood cell lysis assay to assess the C5 inhibitory effect of the parental humanized recombinant chimeric human IgG4 mAb H1-8/L1-9 (VH-11801 (SEQ ID NO:22) and VL-1901 (SEQ ID NO:25)), and its variants IFW PLA (VH-11801 (SEQ ID NO:46) and VL-1901 (SEQ ID NO:25)), FME PLA (SEQ ID NO:51) and VL-1901 (SEQ ID NO:25)), IWW PLA (SEQ ID NO:41) and VL-1901 (SEQ ID NO:25)), and FMW PLA (SEQ ID NO:56) and VL-1901 (SEQ ID NO:25)).

FIG. 35 depicts an Octet tracing of C5 binding and dissociation of the recombinant chimeric human IgG4 mAb H1-8/L1-9, FME, FMEH, FMW, and IFW at pH 5.8 and pH 7.4.

FIG. 36 depicts the % of C5 binding dissociated from peak value for purified recombinant chimeric human IgG4 PLA (SEQ ID NO: 61), mAb H1-8/L1-9, FME, FMEH, FMW, and IFW at pH 5.8 and pH 7.4.

FIG. 37 depicts the results of a classical pathway complement-mediated sheep red blood cell lysis assay to assess the C5 inhibitory effect of recombinant chimeric human IgG4 mAb FME PLA (SEQ ID NO:51) and VL-1901 (SEQ ID NO:25)), FMEH PLA (SEQ ID NO:59) and VL-1901 (SEQ ID NO:25)), FMW PLA (SEQ ID NO:56) and VL-1901 (SEQ ID NO:25)), and IFW PLA (VH-11801 (SEQ ID NO:46) and VL-1901 (SEQ ID NO:25)).

FIG. 38 depicts nucleotide and amino acid sequences of Human IgG4→Fc domain mutations in recombinant chimeric human IgG4 PLA mAbs for PK/PD testing in C5 humanized FcRn/SCID mice.

FIG. 39 depicts the plasma C5 levels in C5 humanized mice produced by hydrodynamic injection of human C5 cDNA.

FIG. 40 depicts the results of an assay assessing the level of total human C5 in the plasma of FcRn/SCID mice genetically modified to express human C5 after injection with recombinant chimeric human IgG4 PLA (SEQ ID NO: 61) mAbs IFW-PLA, FMW-PLA, or FMEH-PLA.

FIG. 41 depicts the results of an assay assessing the level of total IgG4 in the plasma of FcRn/SCID mice genetically modified to express human C5 after injection with recombinant chimeric human IgG4 PLA (SEQ ID NO: 61) mAbs IFW-PLA, FMW-PLA, or FMEH-PLA.

FIG. 42 depicts the results of a classical pathway complement-mediated chicken red blood cell assay assessing the pharmacodynamics of recombinant chimeric human IgG4 PLA (SEQ ID NO: 61) mAbs IFW-PLA, FMW-PLA, or FMEH-PLA in FcRn/SCID mice genetically modified to express human C5.

FIG. 43 depicts three different pathways that can result in a complement activation involving a cascade of target recognition and proteolytic cleavage. All of the pathways converge at the C3 activation step.

FIG. 44 depicts the structure of human FH composed of 20 SCR domains and the structure of a human IgG4 anti-C5 mAb/FH SCR1-5 fusion protein.

FIG. 45 depicts nucleotide and amino acid sequences of VH sequence of mAb H1-8/L1-9 variant FMEH-IgG4PLA-FH1-5, humanized VH-11801 with N→H and L→F substitutions in VH-CDR1, P→M and T→H substitutions in VH-CDR2, and a V→E substitution in VH-CDR3.

FIG. 46 depicts nucleotide and amino acid sequences of VL sequence of mAb H1-8/L1-9 variant FMEH-IgG4PLA-FH1-5, humanized VL-1901 with a Y→H substitution in VL-CDR1.

FIG. 47 depicts SDS gel showing the VH and VL chains of anti-C5 mAb FMEH-IgG4PLA (lane 1) and anti-C5 mAb/FH SCR1-5 fusion protein (FMEH-IgG4PLA-FH1-5) (lane 2).

FIG. 48 depicts an Octet tracing of C5 binding and dissociation of the anti-C5 mAb FMEH-IgG4PLA and the anti-C5 mAb/FH SCR1-5 fusion protein FMEH-IgG4PLA-FH1-5 at pH 5.8 and pH 7.4.

FIG. 49 depicts the % dissociation from peak C5 binding for the anti-C5 mAb FMEH-IgG4 and the anti-C5 mAb/FH SCR1-5 fusion protein, FMEH-IgG4PLA-FH1-5 at pH 7.4 and pH 5.8.

FIG. 50 depicts the results of an alternative pathway complement-mediated rabbit red blood cell lysis assay to assess the inhibitory effect on hemolytic activity of the anti-C5 mAb FMEH-IgG4PLA, anti-C5 mAb/FH SCR1-5 fusion protein, FMEH-IgG4PLA-FH1-5, and benchmark anti-C5 mAbs Eculizumab, and Ravulizumab.

FIG. 51 depicts the level of C3 fragment deposition on non-lysed rabbit RBCs treated with FMEH-IgG4PLA, FMEH-IgG4PLA-FH1-5, Eculizumab, and Ravulizumab.

FIG. 52 depicts the results of an assay assessing the level of human IgG4 in the plasma of C5 humanized FcRn/SCID mice at various time points after injection with FMEH-IgG4PLA, FMEH-IgG4PLA-FH1-5, Eculizumab, and Ravulizumab.

FIG. 53 depicts the result of an assay assessing the levels of FMEH-IgG4PLA-FH1-5 in the plasma of C5 humanized FcRn/SCID mice at various time points after injection by using a detection mAb specific for the human IgG4 moiety of the molecule.

FIG. 54 depicts the result of an assay assessing the levels of FMEH-IgG4PLA-FH1-5 in the plasma of C5 humanized FcRn/SCID mice at various time points after injection by using a detection mAb specific for the FH SCR1-5 moiety of the molecule.

FIG. 55 depicts the results of an ELISA assay assessing the levels of human C5 in the plasma of C5 humanized FcRn/SCID mice at various time points after injection with FMEH-IgG4PLA, FMEH-IgG4PLA-FH1-5, Eculizumab, and Ravulizumab.

FIG. 56 depicts the % of rabbit RBC lysis via alternative complement pathway in a hybrid assay using sera collected from C5 humanized FcRn/SCID mice at various time points after injection with FMEH-IgG4PLA, FMEH-IgG4PLA-FH1-5, Eculizumab, Ravulizumab, and C5-depleted human serum.

FIG. 57 depicts representative results for PNH patient RBC hemolysis in vitro (patient #2). 2E7 PNH patient RBCs in AP buffer were mixed with 35% HCl-acidified normal human serum in the presence of various amount of FMEH-IgG4PLA-FH1-5, Eculizumab, or Ravulizumab. The mixture was incubated at 37° C. for 40 min and hemolysis was readout at OD405. % of lysis was calculated using the formula (OD405(specific ab con.)−OD405(40 mM EDTA))/((OD405(ab con.=0)−OD405(40 mM EDTA))*100%. FMEH-IgG4PLA-FH1-5 more potently blocks PNH patient RBC lysis than Eculizumab and Ravulizumab do in vitro. FIG. 57 depicts the comparative inhibitory activity and IC50 (μg/mL) of FMEH-IgG4PLA-FH1-5, Eculizumab, Ravulizumab, and control IgG4 on alternative pathway complement-mediated lysis of human PNH red blood cells acidified normal human serum (35% AB blood type).

FIG. 58 depicts representative FACS analysis results of C3b coating on PNH patient RBCs following anti-C5 mAb treatment in vitro. Following the hemolysis assay (see FIG. 57 PNH patient RBC hemolysis in vitro), un-lysed RBCs were collected and washed by DPBS. 2E6 cells were incubated with anti-C3b antibody for 30 min on ice followed by FACS analysis. C3b+/CD59− cell population are gated and analyzed. The results are from PNH patient #2. ND means not detectable. FMEH-IgG4PLA-FH1-5 effectively blocks C3b deposition on PNH patient RBC, while Eculizumab and Ravulizumab were not able to block. C3b-opsonized PNH red blood cells (Q4) were detected in samples treated with Eculizumab or Ravulizumab but not with FMEH-IgG4PLA-FH1-5.

FIG. 59 depicts representative results for PNH patient RBC hemolysis in vitro (patient #3). 5E6 PNH RBCs in AP buffer were mixed with 50% HCl-acidified normal human serum in the presence of various amount of FMEH-IgG4PLA-FH1-5, Eculizumab, or Ravulizumab as indicated. The hemolysis was happened at 37° C. for 90 min. and hemolysis was readout using spectrometer at OD405. % of lysis was calculated using the formula (OD405(specific ab con.)−OD405(40 mM EDTA))/((OD405(ab con.=0)−OD405(40 mM EDTA))*100%. FMEH-IgG4PLA-FH1-5 more potently blocks PNH patient RBC lysis than Eculizumab and Ravulizumab do in vitro.

FIG. 60 depicts representative FACS analysis results of C3b coating on PNH patient #3 RBCs following anti-C5 mAb treatment in vitro. Following the hemolysis assay, where 5E6 PNH patient #3 RBCs were missed with 50% HCl-acidified normal human serum and various concentration of specified antibody and incubation, un-lysed RBCs were collected and washed by DPBS. 2E6 un-lysed cells were incubated with anti-C3b antibody for 30 min on ice followed by FACS analysis. C3b+ cell population are gated and analyzed. ND means not detectable. FMEH-IgG4PLA-FH1-5 blocked C3b deposition on PNH patient RBC in a dose dependent manner, while Eculizumab and Ravulizumab were not able to block.

FIG. 61 depicts the effect of FMEH-IgG4PLA and FMEH-IgG4PLA-FH1-5 fusion protein on Cynomolgus monkey alternative pathway complement-mediated lysis. Hemolytic assay of rabbit red blood cells was performed using 50% Cynomolgus monkey serum in the presence of various concentrations of FMEH-IgG4PLA or FMEH-IgG4PLA-FH1-5 fusion proteins. Two benchmark anti-human C5 mAbs with no reactivity to monkey C5 (Eculizumab and Ravulizumab) were used as negative controls.

FIG. 62, comprising FIG. 62A and FIG. 62B, depicts representative Biacore measurement of affinity of FMEH-IgG4PLA to human FcRn at pH 6.0. Purified human FcRn was coupled onto CM5 chip using the amine coupling method (280RU). Biacore analysis was performed on a Biacore-3000 instrument. The chip was regenerated between each binding using 7.4 pH Buffer. The kD of FMHE-IgG4PLA was determined to be 6.47 E-9 M. FIG. 62A depicts representative results demonstrating the FMEH binding to human FcRn. FIG. 62B depicts representative Biacore measurement of affinity of FMEH-IgG4PLA to human FcRn at pH 6.0 and the corresponding analysis.

FIG. 63, comprising FIG. 63A and FIG. 63B, depicts representative Biacore measurement of affinity of FMEH-IgG4PLA-FH1-5 to human FcRn at pH 6.0. Purified human FcRN was coupled onto CM5 chip using the amine coupling method (280RU). Biacore analysis was performed on a Biacore-3000 instrument. The chip was regenerated between each binding using 7.4 pH Buffer. The kD of FMEH-IgG4PLA-FH1-5 was determined to be 8.88 E-9 M. FIG. 63A depicts representative results demonstrating the FMEH-FH1-5 binding to human FcRn. FIG. 63B depicts representative Biacore measurement of affinity of FMEH-IgG4PLA-FH1-5 to human FcRn at pH 6.0 and the corresponding analysis.

FIG. 64, comprising FIG. 64A and FIG. 64B, depicts representative Biacore measurement of affinity of FMEH-IgG4PLA to Cyno FcRn at pH 6.0. Purified Cyno FcRn was coupled onto CM5 chip using the amine coupling method(200RU). Biacore analysis was performed on a Biacore-3000 instrument. The chip was regenerated between each binding using 7.4 pH Buffer. The kD of FMHE-IgG4PLA was determined to be 1.4 E-8 M. FIG. 64A depicts representative results demonstrating FMEH binding to Cyno FcRn. FIG. 64B depicts representative Biacore measurement of affinity of FMEH-IgG4PLA to Cyno FcRn at pH 6.0 and the corresponding analysis.

FIG. 65, comprising FIG. 65A and FIG. 65B, depicts representative Biacore measurement of affinity of FMEH-IgG4PLA-FH1-5 to Cyno FcRn at pH 6.0. Purified Cyno FcRn was coupled onto CM5 chip using the amine coupling method(200RU). Biacore analysis was performed on a Biacore-3000 instrument. The chip was regenerated between each binding using 7.4 pH Buffer. The kD of FMEH-IgG4PLA-FH1-5 was determined to be 3.91 E-8 M. FIG. 65A depicts representative results demonstrating FMEH-FH1-5 binding to Cyno FcRn. FIG. 65B depicts representative Biacore measurement of affinity of FMEH-IgG4PLA-FH1-5 to Cyno FcRn at pH 6.0 and the corresponding results.

FIG. 66 depicts representative pharmacokinetics of FMEH-IgG4PLA-FH1-5 in C5 humanized FcRn/Scid mice (n=2) that was measured by using detection antibodies against human IgG4 Fc or FH fusion protein. In both assays, FMEH-IgG4PLA-FH1-5 in the mouse plasma was captured by an anti-human kappa light chain antibody but different detection antibodies were used for the human IgG4 moiety of the molecule (Left panel) or the human FH1-5 moiety of the molecule (Right panel). Mouse plasma samples were collected at various time points after injection of FMEH-IgG4PLA-FH1-5 as indicated. Both assays demonstrated similar pharmacokinetics, suggesting the anti-C5 mAb and FH1-5 fusion protein maintained its integrity.

FIG. 67, comprising FIG. 67A through FIG. 67C, depicts representative results for characterization of FMEH-FH1-5 by SDS-PAGE and SEC-HPLC. Characterization of FMEH-IgG4PLA-FH1-5 by SDS-PAGE and SEC-HPLC. FMEH-IgG4PLA-FH1-5 expressed from HEK293 cell transfection media was purified using Protein A affinity chromatography. FIG. 67A depicts representative results for reducing SDS-PAGE. In reduced SDS-PAGE, banding at ˜85 KDa and ˜25 kDa are the expected heavy chain-FH1-5 fusion and the light chain, respectively. FIG. 67B depicts representative results for non-reducing SDS-PAGE. In non-reduced SDS-PAGE, the band greater than 200 kDa marker is the expected intact FMEH-IgG4PLA-FH1-5 molecule. FIG. 67C depicts representative results for SEC-HPLC. In SEC-HPLC, FMEH-IgG4PLA-FH1-5 eluted at 6.54 min. and is greater than 95% pure.

FIG. 68 depicts representative results for mass Spectrometer analysis of FMEH-IgG4PLA-FH1-5. FMEH-IgG4PLA-FH1-5 purified from HEK293 transfection cell media by protein A was subjected mass spectrometer analysis to determine molecular weight. Mass of intact, reduced heavy chain, reduced light chain, de-glycosylated intact, reduced and de-glycosylated heavy chain, and reduced and de-glycosylated light chain of FMEH-IgG4PLA-FH1-5 were determined. The experimental mass and theoretic masses match consistently for all samples. The results showed that there were two glycosylation modifications on FMEH-IgG4PLA-FH1-5. Considering canonical N-glycosylation sites in CH2 of Fc, FH domain 1-5 does not appears glycosylated.

FIG. 69, comprising FIG. 69A and FIG. 69B, depicts representative binding affinity of FMEH-IgG4PLA-FH1-5 to human and cyno C5 proteins at pH 7.4 and pH 5.8. FMEH-IgG4PLA-FH1-5 purified from HEK293 transfection cell media was subjected binding affinity determination using a Bio-Layer Interferometry, Gator (Probe Life Inc., China). The experiment was performed under either pH 7.4 or pH 5.8 buffer condition to determine the effect of pH on the binding kinetics. The data were analyzed by the Gator evaluation software (Probe Life Inc., China) using 1:1 binding model. FMEH-IgG4PLA-FH1-5 dissociate from C5 binding complex approximately 10-fold faster under pH 5.8 than pH 7.4. FIG. 69A depicts graphical representation of the binding affinity of FMEH-IgG4PLA-FH1-5 to human and cyno C5 proteins at pH 7.4 and pH 5.8. FIG. 69B depicts tabulated representation of the binding affinity of FMEH-IgG4PLA-FH1-5 to human and cyno C5 proteins at pH 7.4 and pH 5.8.

### DETAILED DESCRIPTION OF THE INVENTION

This invention relates to the inhibition of complement signaling using an anti-C5 antibody, fusion protein, or a combination thereof. The present invention relates, in part, to bi-functional complement inhibitors that can inhibit both C3 and C5 activities and thereby achieve higher potency in treating terminal complement-mediated pathologies as well as provide convenience of less frequent dosing. In some embodiments, the anti-C5 antibody, fusion protein, or a combination thereof, exhibits pH-dependent binding to C5. In some embodiments, the pH-dependent anti-C5 antibody or fusion protein binds more strongly to C5 at a more neutral pH (e.g., about pH 7.4; such as that found in the blood) than it does at a more acidic pH (e.g., about pH 5.8; such as that found in the endosome). In various embodiments, the invention is directed to compositions and methods for treating a complement-mediated disease or complement-mediated disorder in an individual by contacting the individual with an anti-C5 antibody, fusion protein, or a combination thereof. The complement-mediated pathologies and conditions that can be treated with the compositions and methods of the invention include, but are not limited to, MD, AMD, ischemia reperfusion injury, arthritis, rheumatoid arthritis, lupus, ulcerative colitis, stroke, post-surgery systemic inflammatory syndrome, asthma, allergic asthma, COPD, PNH syndrome, myasthenia gravis, NMO, multiple sclerosis, delayed graft function, antibody-mediated rejection, aHUS, CRVO, CRAO, epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, ANCA-mediated glomerulonephritis, lupus nephritis, and combinations thereof), ANCA-mediated vasculitis, Shiga toxin induced HUS, and antiphospholipid antibody-induced pregnancy loss, or any combinations thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The terms “inhibit” and “inhibition,” as used herein, means to reduce, suppress, diminish or block an activity or function by at least about 10% relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%.

The terms “effective amount” and “pharmaceutically effective amount” refer to a sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, and mice and humans.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Operably linked” or “operatively linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “therapeutic treatment” is a treatment administered to a subject who exhibits signs of disease or disorder, for the purpose of diminishing or eliminating those signs.

As used herein, “treating a disease or disorder” means reducing the frequency and/or severity of a sign and/or symptom of the disease or disorder is experienced by a patient.

The phrase “biological sample”, “sample” or “specimen” as used herein, is intended to include any sample comprising a cell, a tissue, or a bodily fluid in which expression of a nucleic acid or polypeptide can be detected. The biological sample may contain any biological material suitable for detecting the desired biomarkers, and may comprise cellular and/or non-cellular material obtained from the individual. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from a subject with heavy chain disease, or prepared by the cloning and expression of VH (variable heavy chain immunoglobulin) genes from a subject.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-C54951).

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “fusion protein” refers a protein created through the attachment of two or more polypeptides which originated from separate proteins. In some embodiments, fusion proteins are created using recombinant DNA technology (e.g., by attaching the nucleic acids that encode each of the parts of the fusion protein) and are typically used in biological research or therapeutics. In some embodiments, fusion proteins are created through chemical conjugation (e.g., covalent conjugation, etc.) with or without a linker between the polypeptides portion of the fusion proteins.

The term “attach” or “attached” as used herein, refers to connecting or uniting by a bond, link, force or tie in order to keep two or more components together, which encompasses either direct or indirect attachment such that, for example, where a first polypeptide is directly bound to a second polypeptide or material, and, for example, where one or more intermediate compounds (e.g., amino acids, peptides, polypeptides, etc.) are disposed between the first polypeptide and the second polypeptide or material.

“CDRs” are defined as the complementarity determining region amino acid sequences of an antibody which are the hypervariable regions of immunoglobulin heavy and light chains See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes and binds to a specific target molecule, but does not substantially recognize or bind other molecules in a sample. In some instances, the terms “specific binding” or “specifically binding,” is used to mean that the recognition and binding is dependent upon the presence of a particular structure (e.g., an antigenic determinant or epitope) on the target molecule. If, for example, an antibody specifically binds to epitope “A,” the presence of an unlabelled molecule containing epitope A (or free, unlabeled A) in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “hybridoma,” as used herein refers to a cell resulting from the fusion of a B-lymphocyte and a fusion partner such as a myeloma cell. A hybridoma can be cloned and maintained indefinitely in cell culture and is able to produce monoclonal antibodies. A hybridoma can also be considered to be a hybrid cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins or variants or fragment thereof, among others. The polypeptides include natural peptides, recombinant peptides, synthetic peptides, or a combination thereof.

The term “progeny” as used herein refers to a descendent or offspring and includes the offspring of a mammal, and also included the differentiated or undifferentiated decedent cell derived from a parent cell. In one usage, the term progeny refers to a descendent cell which is genetically identical to the parent. In another use, the term progeny refers to a descendent cell which is genetically and phenotypically identical to the parent. In yet another usage, the term progeny refers to a descendent cell that has differentiated from the parent cell.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

A “scanning window,” as used herein, refers to a segment of a number of contiguous positions in which a sequence may be evaluated independently of any flanking sequence. A scanning window generally is shifted incrementally along the length of a sequence to be evaluated with each new segment being independently evaluated. An incremental shift may be of 1 or more than one position.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

This invention relates to inhibition of the complement signaling using an anti-C5 antibody or fusion protein thereof. In some embodiments, the invention related to inhibition of the complement signaling using a bi-functional complement inhibitors that can inhibit both C3 and C5 activities. In various embodiments, the invention relates, in part, to methods of treating a complement-mediated disease or complement-mediated disorder in an individual by contacting the individual with an anti-C5 antibody fusion protein thereof.

### Complement Inhibitors and Bi-Functional Antibodies

This invention relates to the inhibition of the complement signaling and complement-related disorders using an anti-human C5 antibody, fusion protein, or an antigen-binding fragment thereof (e.g., anti-C5 antibody or a variant or fragment thereof, anti-C5 fusion protein antibody or a variant or fragment thereof, etc.). In some embodiments, the anti-C5 antibody, fusion protein, or a combination thereof, exhibits bi-functional activity at inhibiting C5 and C3 activation. In some embodiments, the anti-C5 antibody, fusion protein, or a combination thereof, exhibits pH-dependent binding to C5.

In some embodiments, the anti-C5 antibody, fusion protein, or a combination thereof, exhibits pH-dependent binding to C5. In some embodiments, the pH-dependent anti-C5 antibody, fusion protein, or a combination thereof, binds more strongly to C5 at a more neutral pH (e.g., about pH 7.4; such as that found in the blood) than it does at a more acidic pH (e.g., about pH 5.8; such as that found in the endosome). Such pH-dependent binding provides for greater persistence of administered antibody or antibody fusion protein molecules, because immune complexes (i.e., anti-C5 mAb bound to C5 and/or anti-C5 mAb-FH fusion protein bound to C5) taken up by cells will dissociate in the acidic environment of the endosome and allow the freed antibody or antibody-FH fusion protein to be recycled back out of the cell through the neonatal Fc receptor (FcRn) where it is available to bind to a new C5 molecule.

In one embodiment, the invention is directed to inhibiting the complement signaling cascade by specifically targeting complement component C3 protein, or a fragment of the protein C3a or C3b. In one embodiment, the invention is directed to inhibiting the complement signaling cascade by specifically targeting complement component C5 protein, or a fragment of the protein C5a or C5b. In one embodiment, the invention is directed to inhibiting the complement signaling cascade by specifically targeting complement component C3 protein, fragment of the protein C3a or C3b, C5 protein, fragment of the protein C5a or C5b, or any combination thereof. In one embodiment, the invention is directed to methods of treating and preventing inflammation and autoimmune diseases mediated by unwanted, uncontrolled, excessive complement activation. In one embodiment the invention is directed towards the treatment of complement-mediated disease or complement-mediated disorder in an individual by contacting the individual with an anti-C5 antibody. In one embodiment the invention is directed towards the treatment of complement-mediated disease or complement-mediated disorder in an individual by contacting the individual with an anti-C5 fusion protein antibody. In one embodiment the invention is directed towards the treatment of complement-mediated disease or complement-mediated disorder in an individual by contacting the individual with an anti-C5 antibody or an anti-C5 fusion protein antibody.

In one embodiment, the invention is a method of treating a complement-mediated disease or disorder in an individual, comprising the step of administering to said individual an anti-C5 antibody, fusion protein, or a combination thereof, thereby inhibiting the generation of a C3a or C3b protein, and formation of MAC. In one embodiment, the invention is a method of treating a complement-mediated disease or disorder in an individual, comprising the step of administering to said individual an anti-C5 antibody, fusion protein, or a combination thereof, thereby inhibiting the generation of a C5a or C5b protein, and formation of MAC. In one embodiment, the invention is a method of treating a complement-mediated disease or disorder in an individual, comprising the step of administering to said individual an anti-C5 antibody, fusion protein, or a combination thereof, thereby inhibiting the generation of a C3a or C3b protein, inhibiting the generation of a C5a or C5b protein, and formation of MAC. Examples of complement-mediated pathologies that can be treated using the methods of the invention include, but are not limited to MD, AMD, ischemia reperfusion injury, arthritis, rheumatoid arthritis, lupus, ulcerative colitis, stroke, post-surgery systemic inflammatory syndrome, asthma, allergic asthma, COPD, PNH syndrome, myasthenia gravis, NMO, multiple sclerosis, delayed graft function, antibody-mediated rejection, aHUS, CRVO, CRAO, epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, ANCA-mediated glomerulonephritis, lupus nephritis, and combinations thereof), ANCA-mediated vasculitis, Shiga toxin induced HUS, and antiphospholipid antibody-induced pregnancy loss, or any combinations thereof. In some embodiments, the compositions and methods of the invention are useful for treating subject, including subjects having PNH, who are not adequately responsive to treatment with Eculizumab or Ravulizumab. By way of non-limiting example, some subjects may have a mutation in the alpha chain of C5 that may render them resistant to treatment of Eculizumab or Ravulizumab (see Genetic variants in C5 and poor response to eculizumab. Nishimura J, et al., N Engl J Med. 2014 Feb. 13; 370(7):632-9).

The ability of the immune system to discriminate between “self” and “non-self” antigens is vital to the functioning of the immune system as a specific defense against invading microorganisms. “Non-self” antigens are those antigens on substances entering or present in the body which are detectably different or foreign from the subject's own constituents, whereas “self” antigens are those which, in the healthy subject, are not detectably different or foreign from its own constituents. In various embodiments of the methods, the complement activation that is inhibited is that which was triggered by at least one of the group consisting of a microbial antigen, a non-biological foreign surface, altered self-tissue, or combinations thereof. One example of a non-biological foreign surface is blood tubing such as that used in cardio-pulmonary bypass surgery or kidney dialysis. Examples of altered self-tissues include apoptotic, necrotic and ischemia-stressed tissues and cells, tissues and cells devoid of functional complement regulating proteins, or combinations thereof.

In some embodiments, the anti-C5 antibodies (e.g., anti-C5 antibody, anti-C5 fusion protein antibody, antibody fragment, antibody variant, etc.) of the invention inhibit the downstream effects of activation of the alternative complement pathway (AP), the classical pathway (CP), or the lectin pathway (LP). Generally, the CP is initiated by antigen-antibody complexes, the LP is activated by binding of lectins to sugar molecules on microbial surfaces, while the AP is constitutively active at a low level but can be quickly amplified on bacterial, viral, and parasitic cell surfaces due to the lack of regulatory proteins. Host cells are usually protected from AP complement activation by regulatory proteins. But in some situations, such as when the regulatory proteins are defective or missing, the AP can also be activated uncontrollably on host cells, leading to complement-mediated disease or disorder. The CP consists of components C1, C2, C4 and converges with the AP at the C3 activation step. The LP consists of mannose-binding lectins (MBLs) and MBL-associated serine proteases (MASPs) and shares with the CP the components C4 and C2. The AP consists of components C3 and several factors, such as factor B, factor D, properdin, C5 and the fluid phase regulator FH. Complement activation consists of three stages: (a) recognition, (b) enzymatic activation, and (c) membrane attack leading to cell death. The first phase of CP complement activation begins with C1. C1 is made up of three distinct proteins: a recognition subunit, C1 q, and the serine protease subcomponents, C1r and C1s, which are bound together in a calcium-dependent tetrameric complex, C1r2 s2. An intact C1 complex is necessary for physiological activation of C1 to result. Activation occurs when the intact C1 complex binds to immunoglobulin complexed with antigen. This binding activates C1s which then cleaves both the C4 and C2 proteins to generate C4a and C4b, as well as C2a and C2b. The C4b and C2a fragments combine to form the C3 convertase, C4b2a, which in turn cleaves C3 to form C3a and C3b. Activation of the LP is initiated by MBL binding to certain sugars on the target surface and this triggers the activation of MASPs which then cleave C4 and C2 in a manner analogous to the activity of C1s of the CP, resulting in the generation of the C3 convertase, C4b2a. Thus, the CP and LP are activated by different mechanisms but they share the same components C4 and C2 and both pathways lead to the generation of the same C3 convertase, C4b2a. The cleavage of C3 by C4b2a into C3b and C3a is a central event of the complement pathway for two reasons. It initiates the AP amplification loop because surface deposited C3b is a central intermediate of the AP. Both C3a and C3b are biologically important. C3a is proinflammatory and together with C5a are referred to as anaphylatoxins. C3b and its further cleavage products also bind to complement receptors present on neutrophils, eosinophils, monocytes and macrophages, thereby facilitating phagocytosis and clearance of C3b-opsonized particles. Finally, C3b can associate with C4b2a to form the C5 convertase of the CP and LP to activate the terminal complement sequence, leading to the production of C5a, a potent proinflammatory mediator, and the assembly of the lytic MAC, C5-C9.

In one embodiment, the activity of the complement pathway that is inhibited using a method of the invention is complement pathway activation induced by at least one of the group selected from a lipopolysacchride (LPS), lipooligosaccharide (LOS), pathogen-associated molecular patterns (PAMPs) and danger-associated molecular patterns (DAMPs). In another embodiment, the activity of complement signaling that is inhibited using a method of invention is the generation of C5a protein. In another embodiment, the activity of complement signaling that is inhibited using a method of invention is the generation of C5b protein. In another embodiment, the activity of complement signaling that is inhibited using a method of invention is the generation of C3a protein. In another embodiment, the activity of complement signaling that is inhibited using a method of invention is the generation of C3b protein. In various embodiments, the activity of complement signaling that is inhibited using a method of invention is the generation of C5a protein, C5b protein, C3a protein, C3b protein, or any combination thereof. In another embodiment, the activity of complement signaling that is inhibited using a method of the invention is the formation of MAC. In another embodiment, the activity of the complement pathway that is inhibited using a method of the invention is C5 dependent. In yet another embodiment, the activity of the complement pathway that is inhibited using a method of the invention is C3 dependent. In yet another embodiment, the activity of the complement pathway that is inhibited using a method of the invention is C3 dependent, C5 dependent, or both.

In one embodiment, the invention is a method of inhibiting initiation of terminal complement activation in an individual, comprising the step of administering to said individual an anti-C5 antibody, fusion protein, or a combination thereof, thereby inhibiting initiation of terminal complement activation originating from CP, LP or AP activation in an individual. Examples of these embodiments are PNH patients who suffer from complement-mediated hemolysis and individuals suffering from complement-mediated aHUS, asthma, ischemic/reperfusion injury, rheumatoid arthritis and ANCA-mediated kidney diseases. In various embodiments of the invention, diseases and disorders that can be treated using the compositions and methods of the invention include, but are not limited to, complement-mediated hemolysis, complement-mediated aHUS, C3 glomerulopathy, neuromyelitis optica, myasthenia gravis, asthma, ischemic/reperfusion injury, rheumatoid arthritis and ANCA-mediated kidney diseases or disorders.

In various other embodiments, provided herein are methods of identifying a potential anti-C5 antibody, fusion protein, or a combination thereof, having inhibitory effects on terminal complement activation. One such method is the sheep red blood cell lysis assay as described below. Briefly, sheep RBCs (1×10⁷ cells per assay sample prepared in PBS, Complement Technology Inc) were incubated at 37° C. for 20 min with 50% normal human serum (NHS, from Complement Technology Inc) in gelatin veronal buffer (GVB2+, Sigma; total assay volume: 100 μl). Before addition to the sheep RBCs, NHS was pre-incubated with anti-C5 mAbs for 1 hr at 4° C. Lysis reaction was stopped by addition of ice-cold 40 mM EDTA in PBS. The incubation mixtures were centrifuged for 5 min at 1500 rpm and the supernatant was collected and measured for OD405 nm. Samples without NHS or with EDTA added were used as negative lysis controls, and a sample of sheep RBCs lysed completely with distilled water was used as a positive control (100% lysis) against which % lysis in other samples was normalized. A separate method that can be used in confirmatory screening of anti-human C5 blocking mAbs includes the steps of: a) coating a plate with LPS; b) washing the plate to remove unbound LPS; c) adding bovine serum albumin (BSA) in phosphate buffered saline (PBS); d) washing the plate to remove unbound BSA; e) adding a mixture of a candidate anti-C5 antibody compound that has been pre-incubated with serum and is mixed into normal human serum; f) washing the plate; g) adding an HRP-conjugated anti-human C5b-9 or anti-human C6 antibody (anti-human TCC antibody, clone aE11 or biotin-labeled anti-human C6 antibody, both from Quidel); h) washing the plate to remove unbound antibody; i) adding HRP Substrate Reagent; j) adding sulphuric acid to stop the reaction; k) measuring the optical density at 450 nm; 1) comparing the optical density of the plate containing the candidate anti-C5 antibody compound to the optical density of a positive comparator control and a negative comparator control; wherein when the optical density is diminished as compared with the positive comparator control, the anti-C5 antibody is identified.

### Anti-C5 Antibodies and Anti-C5 Fusion Protein Antibodies

In some embodiments, the invention includes compositions comprising an antibody or a fusion protein that specifically inhibits the activation of C3 by C3 convertases. In some embodiments, the invention includes compositions comprising an antibody or a fusion protein that specifically binds to and inhibits C5 activation. In some embodiments, the invention includes compositions comprising an antibody or a fusion protein that specifically inhibits activation of C3, C5, or both. In some embodiments, the anti-C5 antibody or the fusion protein is a polyclonal antibody, monoclonal antibody, chimeric antibody, or humanized antibody.

In some embodiments, the antibody or the fusion protein is an antibody fragment. In some embodiments, the C3 is human C3. In some embodiments, the C5 is human C5. In some embodiments, the anti-C5 antibody fusion protein is an anti-C5 mAb linked to FH. In some embodiments, the anti-C5 antibody fusion protein is an anti-C5 mAb linked to a fragment of FH. In some embodiments, the FH is human FH. In some embodiments, the anti-C5 antibody or the fusion protein exhibits pH-dependent binding to C5. In some embodiments, the pH-dependent anti-C5 antibody or the fusion protein binds more strongly to C5 at a more neutral pH (e.g., about pH 7.4; such as that found in the blood) than it does at a more acidic pH (e.g., about pH 5.8; such as that found in the endosome).

In some embodiments, binding of the antibody, fusion protein, or the fragment of the antibody to human-C5 is associated with a reduction in the generation of C3a or C3b and the formation of MAC in the complement activation pathway in an intact organism. In some embodiments, binding of the antibody, fusion protein, or the fragment of the antibody to human-C5 is associated with a reduction in the generation of C5a or C5b and the formation of MAC in the complement activation pathway in an intact organism.

In some embodiments, the invention is a protein or a polypeptide capable of inhibiting to the activation of human C3. In some embodiments, the invention is a protein or a polypeptide capable of binding to and inhibiting the activation of human C5. In some embodiments, the invention is a protein or a polypeptide capable of binding to and/or inhibiting the activation of human C3, human C5, or both. In some embodiments, the antibody, fusion protein, or antibody fragment; the protein or the polypeptide binds to a relevant portion or fraction or epitope of the human-C3 convertases; and the interactions of the antibody, fusion protein, or the antibody fragment thereof, or the protein or the polypeptide interacts with the relevant portion of the human-C3 convertases is associated with a reduction in the generation of C3a or C3b and the formation of MAC in an intact organism. In some embodiments, the antibody, fusion protein, or antibody fragment; the protein or the polypeptide binds to a relevant portion or fraction or epitope of the human-C5; and the binding of the antibody, fusion protein, or the antibody fragment thereof, or the protein or the polypeptide to the relevant portion of the human-C5 is associated with a reduction in the generation of C5a or C5b and the formation of MAC in an intact organism.

In some embodiments, the antibody, fusion protein, or a fragment thereof with inhibitory activity for human C3 convertases, is further conjugated to a protein, a peptide or another compound. In some embodiments, the human-C3 convertase-inhibiting antibody, fusion protein, or an antibody fragment thereof, is conjugated to a protein, a peptide or other compound. In some embodiments, the protein, peptide or other compound to which the human-C3 convertase-inhibiting antibody, fusion protein, or antibody fragment thereof is conjugated is a targeting moiety (i.e., the targeting moiety specifically binds to a molecule other than human-C3). In some embodiments, the protein, peptide, or other compound to which the human-C3 convertase-inhibiting antibody, fusion protein, or antibody fragment thereof is conjugated to is an effector molecule (e.g., a cytotoxic molecule).

In some embodiments, the human-C5 binding antibody, fusion protein, or a C5 binding antibody fragment thereof, is further conjugated to a protein, a peptide or another compound. In some embodiments, the human-C5 binding antibody, fusion protein, or an antibody fragment thereof, is conjugated to a protein, a peptide or other compound. In some embodiments, the protein, peptide or other compound to which the human-C5 binding antibody, fusion protein, or antibody fragment thereof is conjugated is a targeting moiety (i.e., the targeting moiety specifically binds to a molecule other than human-C5). In some embodiments, the protein, peptide, or other compound to which the human-C5 binding antibody, fusion protein, or antibody fragment thereof is conjugated to is an effector molecule (e.g., a cytotoxic molecule).

In some embodiments, the human-C3 convertase-inhibiting and -C5 binding antibody, fusion protein, or a C3 convertase-inhibiting and C5 binding antibody fragment thereof, is further conjugated to a protein, a peptide or another compound. In some embodiments, the human-C3 convertase-inhibiting and -C5 binding antibody, fusion protein, or an antibody fragment thereof, is conjugated to a protein, a peptide or other compound. In some embodiments, the protein, peptide or other compound to which the human-C3 convertase-inhibiting and -C5 binding antibody, fusion protein, or antibody fragment thereof is conjugated is a targeting moiety (i.e., the targeting moiety specifically binds to a molecule other than human-C3 and -C5). In some embodiments, the protein, peptide, or other compound to which the human-C3 convertase-inhibiting and -C5 binding antibody, fusion protein, or antibody fragment thereof is conjugated to is an effector molecule (e.g., a cytotoxic molecule).

In various embodiments, the antibody is a fusion protein. In one embodiment, the fusion protein comprises a complement control protein. In one embodiment, the fusion protein comprises a CR1 or a fragment thereof. In one embodiment, the fusion protein comprises a MCP or a fragment thereof. In one embodiment, the fusion protein comprises a C4BP or a fragment thereof. In one embodiment, the fusion protein comprises a DAF or a fragment thereof. In one embodiment, the fusion protein comprises an ApoE or a fragment thereof. In one embodiment, the fusion protein comprises a FH protein or a fragment thereof. In one embodiment, the fusion protein comprises a human IgG4 or a fragment thereof. In one embodiment, the fusion protein comprises a linker. In various embodiments, the fusion protein comprises a CR1 or a fragment thereof, a MCP or a fragment thereof, a C4BP or a fragment thereof, a DAF or a fragment thereof, an ApoE or a fragment thereof, a FH protein or a fragment thereof, a human IgG4 or a fragment thereof, a linker, or any combination thereof.

In one embodiment, the fragment of FH comprises at least one SCR domains of the FH protein. In one embodiment, the fragment of FH comprises at least two SCR domains of the FH protein. In one embodiment, the fragment of FH comprises at least three SCR domains of the FH protein. In one embodiment, the fragment of FH comprises at least four SCR domains of the FH protein. In one embodiment, the fragment of FH comprises at least five SCR domains of the FH protein. In one embodiment, the fragment of FH comprises at least six SCR domains of the FH protein. In one embodiment, the fragment of FH comprises at least seven SCR domains of the FH protein. In one embodiment, the fragment of FH comprises at least eight SCR domains of the FH protein. In one embodiment, the fragment of FH comprises at least nine SCR domains of the FH protein. In one embodiment, the fragment of FH comprises at least ten SCR domains of the FH protein. In one embodiment, the fragment of FH comprises at least eleven SCR domains of the FH protein. In one embodiment, the fragment of FH comprises at least twelve SCR domains of the FH protein. In one embodiment, the fragment of FH comprises at least thirteen SCR domains of the FH protein. In one embodiment, the fragment of FH comprises at least fourteen SCR domains of the FH protein. In one embodiment, the fragment of FH comprises at least fifteen SCR domains of the FH protein. In one embodiment, the fragment of FH comprises at least sixteen SCR domains of the FH protein. In one embodiment, the fragment of FH comprises at least seventeen SCR domains of the FH protein. In one embodiment, the fragment of FH comprises at least eighteen SCR domains of the FH protein. In one embodiment, the fragment of FH comprises at least nineteen SCR domains of the FH protein. In one embodiment, the fragment of FH comprises at least twenty SCR domains of the FH protein.

In one embodiment, the fragment of FH comprises SCR domains 1-20 of the FH protein. In one embodiment, the fragment of FH comprises SCR domains 1-5 of the FH protein. In one embodiment, the fragment of FH comprises SCR domains 2-5 of the FH protein. In one embodiment, the fragment of FH comprises SCR domains 3-5 of the FH protein. In one embodiment, the fragment of FH comprises SCR domains 4 and 5 of the FH protein. In one embodiment, the fragment of FH comprises SCR domain 5 of the FH protein.

In one embodiment, the fusion protein comprises the antibody and a fusion protein partner (e.g., FH or a functional fragment thereof). In one embodiment, the fusion protein comprises a fusion protein partner (e.g., FH or a functional fragment thereof) bound to the antibody. In some embodiments, the fusion protein comprises fusion protein partner (e.g., FH or a functional fragment thereof) fused (e.g., bound) with the antibody, either directly or via a linker. In one embodiment, the fusion protein comprises a fusion protein partner (e.g., FH or a functional fragment thereof) bound to the antibody with at least one linker. In one embodiment, the linker is not a cleavable linker. In one embodiment, the linker is a cleavable linker. For example, in some embodiment, the cleavable linker is a chemically cleavable linker, enzyme cleavable linker, peptide-based linker, or any combination thereof.

In some embodiments, the chemically cleavable linker is an acid-cleavable linkers or reducible linker. In one embodiment, the acid-cleavable linker is specifically designed to remain stable at the neutral pH of blood circulation, but undergo hydrolysis and release the cytotoxic drug in the acidic environment of the cellular compartments. In one embodiment, the reducible linker is designed to remain stable in the oxygen-rich environment in the bloodstream, and is selectively cleaved in the reducing environment of the cell. In another embodiment, the peptide-based linker is designed to keep intact in systemic circulation, and to be cleaved by specific intracellular proteases, such as cathepsin B. Examples of linkers include, but are not limited to, linkers containing a lysosomal-specific protease cleavage site, linkers containing mixed disulfides, aminoethoxyethoxyacetate (AEEA), β-glucuronide linker, peptide linkers cleavable by intracellular proteases, e.g., lysosomal proteases or endosomal proteases, dipeptide linker (e.g., valine-citrulline (val-cit) or a phenylalanine-lysine (phe-lys) linker), aminohexanoate, hydrazone, thiomaleimide, and dibenzocyclooctyne (DBCO).

In one embodiment, the fusion protein comprises a fusion protein partner (e.g., FH or a functional fragment thereof) bound to the antibody without a linker. In one embodiment, the fusion protein comprises a fusion protein partner (e.g., FH or a functional fragment thereof) bound to C-terminal of the antibody. In one embodiment, the fusion protein comprises a fusion protein partner (e.g., FH or a functional fragment thereof) bound to N-terminal of the antibody.

In one embodiment, the fusion protein comprises a fusion protein partner (e.g., FH or a functional fragment thereof) bound to the anti-C5 mAb. In one embodiment, the fusion protein comprises a fusion protein partner (e.g., FH or a functional fragment thereof) bound to the anti-C5 mAb with at least one linker.

In one embodiment, the fusion protein comprises a fusion protein partner (e.g., FH or a functional fragment thereof) bound to the anti-C5 mAb without a linker. In one embodiment, the fusion protein comprises a fusion protein partner (e.g., FH or a functional fragment thereof) bound to C-terminal of the anti-C5 mAb. In one embodiment, the fusion protein comprises a fusion protein partner (e.g., FH or a functional fragment thereof) bound to N-terminal of the anti-C5 mAb.

In one embodiment, the fusion protein comprises a fusion protein partner (e.g., FH or a functional fragment thereof) bound to a variant of the antibody. For example, in one embodiment, the fusion protein comprises a fusion protein partner (e.g., FH or a functional fragment thereof) bound to a variant of the anti-C5 mAb. In one embodiment, the fusion protein comprises a fusion protein partner (e.g., FH or a functional fragment thereof) bound to a variant of the antibody with at least one linker.

In one embodiment, the fusion protein comprises a fusion protein partner (e.g., FH or a functional fragment thereof) bound to a variant of the antibody without a linker. In one embodiment, the fusion protein comprises a fusion protein partner (e.g., FH or a functional fragment thereof) bound to C-terminal of a variant of the antibody. In one embodiment, the fusion protein comprises a fusion protein partner (e.g., FH or a functional fragment thereof) bound to N-terminal of a variant of the antibody.

In one embodiment, the fusion protein comprises a fusion protein partner (e.g., FH or a functional fragment thereof) fused (e.g., bound) to a fragment of the antibody. In one embodiment, the fusion protein comprises a fusion protein partner (e.g., FH or a functional fragment thereof) fused to a fragment of the antibody with or without a linker. For example, in one embodiment, the fusion protein comprises a fusion protein partner (e.g., FH or a functional fragment thereof) fused to a fragment of the anti-C5 mAb. In one embodiment, the fusion protein comprises a fusion protein partner (e.g., FH or a functional fragment thereof) fused to a fragment of the antibody with at least one linker.

In one embodiment, the fusion protein comprises a fusion protein partner (e.g., FH or a functional fragment thereof) fused to a fragment of the antibody without a linker. In one embodiment, the fusion protein comprises a fusion protein partner (e.g., FH or a functional fragment thereof) fused to C-terminal of a fragment of the antibody. In one embodiment, the fusion protein comprises a fusion protein partner (e.g., FH or a functional fragment thereof) fused to N-terminal of a fragment of the antibody.

In one embodiment, the fusion protein comprises a fusion protein partner (e.g., FH or a functional fragment thereof) fused to a VH sequence of the antibody. For example, in one embodiment, the fusion protein comprises a fusion protein partner (e.g., FH or a functional fragment thereof) fused to a VH sequence of the anti-C5 mAb. In one embodiment, the fusion protein comprises a fusion protein partner (e.g., FH or a functional fragment thereof) fused to a VH sequence of the antibody with at least one linker.

In one embodiment, the fusion protein comprises a fusion protein partner (e.g., FH or a functional fragment thereof) fused to a VH sequence of the antibody without a linker. In one embodiment, the fusion protein comprises a fusion protein partner (e.g., FH or a functional fragment thereof) fused to C-terminal of a VH sequence of the antibody. In one embodiment, the fusion protein comprises a fusion protein partner (e.g., FH or a functional fragment thereof) fused to to N-terminal of a VH sequence of the antibody.

In one embodiment, the fusion protein comprises a fusion protein partner (e.g., FH or a functional fragment thereof) fused to a VL sequence of the antibody. For example, in one embodiment, the fusion protein comprises a fusion protein partner (e.g., FH or a functional fragment thereof) fused to a VL sequence of the anti-C5 mAb. In one embodiment, the fusion protein comprises a fusion protein partner (e.g., FH or a functional fragment thereof) fused to a VL sequence of the antibody with at least one linker.

In one embodiment, the fusion protein comprises a fusion protein partner (e.g., FH or a functional fragment thereof) fused to a VL sequence of the antibody without a linker. In one embodiment, the fusion protein comprises a fusion protein partner (e.g., FH or a functional fragment thereof) fused to C-terminal of a VL sequence of the antibody. In one embodiment, the fusion protein comprises a fusion protein partner (e.g., FH or a functional fragment thereof) fused to N-terminal of a VL sequence of the antibody.

In one embodiment, the anti-C5 antibody, fusion protein, or an antigen-binding fragment thereof comprises at least one of the CDRs selected from the group consisting of: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:3 or a variant thereof comprising up to about 3 (such as about any of 1, 2, or 3) amino acid substitutions; and VL-CDR1: SEQ ID NO:8, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:3; and VL-CDR1: SEQ ID NO:8.

In some embodiments, the anti-C5 antibody, fusion protein, or antigen-binding fragment thereof comprises: VH-CDR2 comprising the amino acid sequence of SEQ ID NO:4, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:4; and VL-CDR2: SEQ ID NO:9.

In some embodiments, the anti-C5 antibody, fusion protein, or antigen-binding fragment thereof comprises: VH-CDR3 comprising the amino acid sequence of SEQ ID NO:5, or a variant thereof comprising up to about 3 (such as about any of 1, 2, or 3) amino acid substitutions; and VL-CDR3: SEQ ID NO:10, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:5; and VL-CDR3: SEQ ID NO:10.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:3; VH-CDR2 comprising the amino acid sequence of SEQ ID NO:4; VH-CDR3 comprising the amino acid sequence of SEQ ID NO:5; VL-CDR1 comprising the amino acid sequence of SEQ ID NO:8; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or antigen-binding fragment thereof comprises: VH-CDR3 comprising the amino acid sequence of SEQ ID NO:5, or a variant thereof comprising up to about 3 (such as about any of 1, 2, or 3) amino acid substitutions; and VL-CDR3: SEQ ID NO:11, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:5; and VL-CDR3: SEQ ID NO:11.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:11, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:11.

In some embodiments, the anti-C5 antibody, fusion protein, or antigen-binding fragment thereof comprises: VH-CDR1 comprising the amino acid sequence of SEQ ID NO:3; VH-CDR2 comprising the amino acid sequence of SEQ ID NO:4; VH-CDR3 comprising the amino acid sequence of SEQ ID NO:5; VL-CDR1 comprising the amino acid sequence of SEQ ID NO:8; VL-CDR2 comprising the amino acid sequence of SEQ ID NO:9; and VL-CDR3 comprising the amino acid sequence of SEQ ID NO:11.

In some embodiments, the anti-C5 antibody, fusion protein, or an antigen binding fragment thereof comprises a heavy chain comprising the amino acid sequence of SEQ ID NO:2, or a variant thereof. In other embodiments, the anti-C5 antibody comprises a light chain comprising the amino acid sequence of SEQ ID NO:13, or a variant thereof. In another embodiment, the anti-C5 antibody is mAb L3-1, or a variant thereof. The monoclonal anti-C5 antibody mAb L3-1 comprises a heavy chain comprising the amino acid sequence of SEQ ID NO:2 and a light chain comprising the amino acid sequence of SEQ ID NO:13. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:3 or a variant thereof comprising up to about 3 (such as about any of 1, 2, or 3) amino acid substitutions; and VL-CDR1: SEQ ID NO:14, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:3; and VL-CDR1: SEQ ID NO:14.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:14, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:14, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:3; VH-CDR2 comprising the amino acid sequence of SEQ ID NO:4; VH-CDR3 comprising the amino acid sequence of SEQ ID NO:5; VL-CDR1 comprising the amino acid sequence of SEQ ID NO:14; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:2, or a variant thereof. In other embodiments, the anti-C5 antibody comprises a light chain comprising the amino acid sequence of SEQ ID NO:16, or a variant thereof. In another embodiment, the anti-C5 antibody is mAb L1-2, or a variant thereof. The monoclonal anti-C5 antibody mAb L1-2 comprises a heavy chain comprising the amino acid sequence of SEQ ID NO:2 and a light chain comprising the amino acid sequence of SEQ ID NO:16. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:17 or a variant thereof comprising up to about 3 (such as about any of 1, 2, or 3) amino acid substitutions; and VL-CDR1: SEQ ID NO:8, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:17; and VL-CDR1: SEQ ID NO:8.

In some embodiments, the anti-C5 antibody, fusion protein, or antigen-binding fragment thereof comprises: VH-CDR1 comprising the amino acid sequence of SEQ ID NO:17, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:17, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:17; VH-CDR2 comprising the amino acid sequence of SEQ ID NO:4; VH-CDR3 comprising the amino acid sequence of SEQ ID NO:5; VL-CDR1 comprising the amino acid sequence of SEQ ID NO:8; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:19, or a variant thereof. In other embodiments, the anti-C5 antibody comprises a light chain comprising the amino acid sequence of SEQ ID NO:7, or a variant thereof. In another embodiment, the anti-C5 antibody is mAb H1-4, or a variant thereof. The monoclonal anti-C5 antibody mAb H1-4 comprises a heavy chain comprising the amino acid sequence of SEQ ID NO:19 and a light chain comprising the amino acid sequence of SEQ ID NO:7. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:20 or a variant thereof comprising up to about 3 (such as about any of 1, 2, or 3) amino acid substitutions; and VL-CDR1: SEQ ID NO:23, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:20; and VL-CDR1: SEQ ID NO:23.

In some embodiments, the anti-C5 antibody, fusion protein, or antigen-binding fragment thereof comprises: VH-CDR1 comprising the amino acid sequence of SEQ ID NO:20, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:20, or a variant thereof comprising up to about 3 (such as about any of 1, 2, or 3) amino acid substitutions; VH-CDR2 comprising the amino acid sequence of SEQ ID NO:4, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:20; VH-CDR2 comprising the amino acid sequence of SEQ ID NO:4; VH-CDR3 comprising the amino acid sequence of SEQ ID NO:5; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:22, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:22 and a light chain comprising the amino acid sequence of SEQ ID NO:25. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof. In another embodiment, the anti-C5 antibody comprises all of the CDRs of the group consisting of: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof.

In some embodiments, the anti-C5 antibody, fusion protein, or antigen-binding fragment thereof comprises: VH-CDR2 comprising the amino acid sequence of SEQ ID NO:26, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:26; and VL-CDR2: SEQ ID NO:9

In some embodiments, the anti-C5 antibody, fusion protein, or antigen-binding fragment thereof comprises: VH-CDR3 comprising the amino acid sequence of SEQ ID NO:5, or a variant thereof comprising up to about 3 (such as about any of 1, 2, or 3) amino acid substitutions; and VL-CDR3: SEQ ID NO:29, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:5; and VL-CDR3: SEQ ID NO:29.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:26, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:29, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:26, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:29.

In some embodiments, the anti-C5 antibody, fusion protein, or antigen-binding fragment thereof comprises: VH-CDR1 comprising the amino acid sequence of SEQ ID NO:3; VH-CDR2 comprising the amino acid sequence of SEQ ID NO:26; VH-CDR3 comprising the amino acid sequence of SEQ ID NO:5; VL-CDR1 comprising the amino acid sequence of SEQ ID NO:8; VL-CDR2 comprising the amino acid sequence of SEQ ID NO:9; and VL-CDR3 comprising the amino acid sequence of SEQ ID NO:29.

In some embodiments, the anti-C5 antibody, fusion protein, or an antigen binding fragment thereof comprises a heavy chain comprising the amino acid sequence of SEQ ID NO:28, or a variant thereof. In other embodiments, the anti-C5 antibody comprises a light chain comprising the amino acid sequence of SEQ ID NO:31, or a variant thereof. In another embodiment, the anti-C5 antibody is mAb H2-6/L3-5, or a variant thereof. The monoclonal anti-C5 antibody mAb H2-6/L3-5 comprises a heavy chain comprising the amino acid sequence of SEQ ID NO:28 and a light chain comprising the amino acid sequence of SEQ ID NO:31 In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:3; VH-CDR2: SEQ ID NO:34; VH-CDR3: SEQ ID NO:5; VL-CDR1: SEQ ID NO:8; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:3; VH-CDR2: SEQ ID NO:34; VH-CDR3: SEQ ID NO:5; VL-CDR1: SEQ ID NO:8; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR2 comprising the amino acid sequence of SEQ ID NO:34, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:34; and VL-CDR2: SEQ ID NO:9.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:34, or a variant thereof comprising up to about 3 (such as about any of 1, 2, or 3) amino acid substitutions; VH-CDR3 comprising the amino acid sequence of SEQ ID NO:5, or a variant thereof comprising up to about 3 (such as about any of 1, 2, or 3) amino acid substitutions; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:3, or a variant thereof comprising up to about 3 (such as about any of 1, 2, or 3) amino acid substitutions; VH-CDR2 comprising the amino acid sequence of SEQ ID NO:34, or a variant thereof comprising up to about 3 (such as about any of 1, 2, or 3) amino acid substitutions; VH-CDR3 comprising the amino acid sequence of SEQ ID NO:5; VL-CDR1 comprising the amino acid sequence of SEQ ID NO:8, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:3; VH-CDR2 comprising the amino acid sequence of SEQ ID NO:34 VH-CDR3 comprising the amino acid sequence of SEQ ID NO:5; VL-CDR1 comprising the amino acid sequence of SEQ ID NO:8; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:36, or a variant thereof. In other embodiments, the anti-C5 antibody comprises a light chain comprising the amino acid sequence of SEQ ID NO:7, or a variant thereof. In some embodiments, the monoclonal anti-C5 antibody comprises a heavy chain comprising the amino acid sequence of SEQ ID NO:36 and a light chain comprising the amino acid sequence of SEQ ID NO:7. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:37 or a variant thereof comprising up to about 3 (such as about any of 1, 2, or 3) amino acid substitutions; and VL-CDR1: SEQ ID NO:23, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:37; and VL-CDR1: SEQ ID NO:23.

In some embodiments, the anti-C5 antibody, fusion protein, or antigen-binding fragment thereof comprises: VH-CDR2 comprising the amino acid sequence of SEQ ID NO:38, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:38; and VL-CDR2: SEQ ID NO:9.

In some embodiments, the anti-C5 antibody, fusion protein, or antigen-binding fragment thereof comprises: VH-CDR3 comprising the amino acid sequence of SEQ ID NO:39, or a variant thereof comprising up to about 3 (such as about any of 1, 2, or 3) amino acid substitutions; and VL-CDR3: SEQ ID NO:10, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:39; and VL-CDR3: SEQ ID NO:10.

In some embodiments, the anti-C5 antibody, fusion protein, or antigen-binding fragment thereof comprises: VH-CDR1 comprising the amino acid sequence of SEQ ID NO:37, or a variant thereof comprising up to about 3 (such as about any of 1, 2, or 3) amino acid substitutions; VH-CDR2 comprising the amino acid sequence of SEQ ID NO:38, or a variant thereof comprising up to about 3 (such as about any of 1, 2, or 3) amino acid substitutions; VH-CDR3 comprising the amino acid sequence of SEQ ID NO:39, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:37, or a variant thereof comprising up to about 3 (such as about any of 1, 2, or 3) amino acid substitutions; VH-CDR2 comprising the amino acid sequence of SEQ ID NO:38, or a variant thereof comprising up to about 3 (such as about any of 1, 2, or 3) amino acid substitutions; VH-CDR3 comprising the amino acid sequence of SEQ ID NO:39; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:37; VH-CDR2 comprising the amino acid sequence of SEQ ID NO:38; VH-CDR3 comprising the amino acid sequence of SEQ ID NO:39; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:41, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:41 and a light chain comprising the amino acid sequence of SEQ ID NO:25. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:42 or a variant thereof comprising up to about 3 (such as about any of 1, 2, or 3) amino acid substitutions; and VL-CDR1: SEQ ID NO:23, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:42; and VL-CDR1: SEQ ID NO:23.

In some embodiments, the anti-C5 antibody, fusion protein, or antigen-binding fragment thereof comprises: VH-CDR2 comprising the amino acid sequence of SEQ ID NO:43, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:43; and VL-CDR2: SEQ ID NO:9.

In some embodiments, the anti-C5 antibody, fusion protein, or antigen-binding fragment thereof comprises: VH-CDR3 comprising the amino acid sequence of SEQ ID NO:44, or a variant thereof comprising up to about 3 (such as about any of 1, 2, or 3) amino acid substitutions; and VL-CDR3: SEQ ID NO:10, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:44; and VL-CDR3: SEQ ID NO:10.

In some embodiments, the anti-C5 antibody, fusion protein, or antigen-binding fragment thereof comprises: VH-CDR1 comprising the amino acid sequence of SEQ ID NO:42, or a variant thereof comprising up to about 3 (such as about any of 1, 2, or 3) amino acid substitutions; VH-CDR2 comprising the amino acid sequence of SEQ ID NO:43, or a variant thereof comprising up to about 3 (such as about any of 1, 2, or 3) amino acid substitutions; VH-CDR3 comprising the amino acid sequence of SEQ ID NO:44, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:42, or a variant thereof comprising up to about 3 (such as about any of 1, 2, or 3) amino acid substitutions; VH-CDR2 comprising the amino acid sequence of SEQ ID NO:43, or a variant thereof comprising up to about 3 (such as about any of 1, 2, or 3) amino acid substitutions; VH-CDR3 comprising the amino acid sequence of SEQ ID NO:44; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:42; VH-CDR2 comprising the amino acid sequence of SEQ ID NO:43; VH-CDR3 comprising the amino acid sequence of SEQ ID NO:44; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:46, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:46 and a light chain comprising the amino acid sequence of SEQ ID NO:25. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:47 or a variant thereof comprising up to about 3 (such as about any of 1, 2, or 3) amino acid substitutions; and VL-CDR1: SEQ ID NO:23, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:47; and VL-CDR1: SEQ ID NO:23.

In some embodiments, the anti-C5 antibody, fusion protein, or antigen-binding fragment thereof comprises: VH-CDR2 comprising the amino acid sequence of SEQ ID NO:48, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:48; and VL-CDR2: SEQ ID NO:9.

In some embodiments, the anti-C5 antibody, fusion protein, or antigen-binding fragment thereof comprises: VH-CDR3 comprising the amino acid sequence of SEQ ID NO:49, or a variant thereof comprising up to about 3 (such as about any of 1, 2, or 3) amino acid substitutions; and VL-CDR3: SEQ ID NO:10, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:49; and VL-CDR3: SEQ ID NO:10.

In some embodiments, the anti-C5 antibody, fusion protein, or antigen-binding fragment thereof comprises: VH-CDR1 comprising the amino acid sequence of SEQ ID NO:47, or a variant thereof comprising up to about 3 (such as about any of 1, 2, or 3) amino acid substitutions; VH-CDR2 comprising the amino acid sequence of SEQ ID NO:48, or a variant thereof comprising up to about 3 (such as about any of 1, 2, or 3) amino acid substitutions; VH-CDR3 comprising the amino acid sequence of SEQ ID NO:49, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:47, or a variant thereof comprising up to about 3 (such as about any of 1, 2, or 3) amino acid substitutions; VH-CDR2 comprising the amino acid sequence of SEQ ID NO:48, or a variant thereof comprising up to about 3 (such as about any of 1, 2, or 3) amino acid substitutions; VH-CDR3 comprising the amino acid sequence of SEQ ID NO:49; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:47; VH-CDR2 comprising the amino acid sequence of SEQ ID NO:48; VH-CDR3 comprising the amino acid sequence of SEQ ID NO:49; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:51, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:51 and a light chain comprising the amino acid sequence of SEQ ID NO:25. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:52; VH-CDR2: SEQ ID NO:53; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:52; VH-CDR2: SEQ ID NO:53; VH-CDR3: SEQ ID NO:54; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR1 comprising the amino acid sequence of SEQ ID NO:52 or a variant thereof comprising up to about 3 (such as about any of 1, 2, or 3) amino acid substitutions; and VL-CDR1: SEQ ID NO:23, or a variant thereof comprising up to about 3 (such as about any of 1, 2, or 3) amino acid substitutions. In some embodiments, the anti-C5 antibody or an antigen-binding fragment thereof comprises: VH-CDR1 comprising the amino acid sequence of SEQ ID NO:52; and VL-CDR1: SEQ ID NO:23.

In some embodiments, the anti-C5 antibody, fusion protein, or antigen-binding fragment thereof comprises: VH-CDR2 comprising the amino acid sequence of SEQ ID NO:53, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:53; and VL-CDR2: SEQ ID NO:9.

In some embodiments, the anti-C5 antibody, fusion protein, or antigen-binding fragment thereof comprises: VH-CDR3 comprising the amino acid sequence of SEQ ID NO:54, or a variant thereof comprising up to about 3 (such as about any of 1, 2, or 3) amino acid substitutions; and VL-CDR3: SEQ ID NO:10, or a variant thereof comprising up to about 3 (such as about any of 1, 2, or 3) amino acid substitutions. In some embodiments, the anti-C5 antibody or an antigen-binding fragment thereof comprises: VH-CDR3 comprising the amino acid sequence of SEQ ID NO:54; and VL-CDR3: SEQ ID NO:10.

In some embodiments, the anti-C5 antibody, fusion protein, or antigen-binding fragment thereof comprises: VH-CDR1 comprising the amino acid sequence of SEQ ID NO:52, or a variant thereof comprising up to about 3 (such as about any of 1, 2, or 3) amino acid substitutions; VH-CDR2 comprising the amino acid sequence of SEQ ID NO:53, or a variant thereof comprising up to about 3 (such as about any of 1, 2, or 3) amino acid substitutions; VH-CDR3 comprising the amino acid sequence of SEQ ID NO:54, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:52, or a variant thereof comprising up to about 3 (such as about any of 1, 2, or 3) amino acid substitutions; VH-CDR2 comprising the amino acid sequence of SEQ ID NO:53, or a variant thereof comprising up to about 3 (such as about any of 1, 2, or 3) amino acid substitutions; VH-CDR3 comprising the amino acid sequence of SEQ ID NO:54; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:52; VH-CDR2 comprising the amino acid sequence of SEQ ID NO:53; VH-CDR3 comprising the amino acid sequence of SEQ ID NO:54; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:56, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:56 and a light chain comprising the amino acid sequence of SEQ ID NO:25. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:47; VH-CDR2: SEQ ID NO:57; VH-CDR3: SEQ ID NO:49; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:47; VH-CDR2: SEQ ID NO:57; VH-CDR3: SEQ ID NO:49; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR2 comprising the amino acid sequence of SEQ ID NO:57, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:57; and VL-CDR2: SEQ ID NO:9.

In some embodiments, the anti-C5 antibody, fusion protein, or antigen-binding fragment thereof comprises: VH-CDR1 comprising the amino acid sequence of SEQ ID NO:47, or a variant thereof comprising up to about 3 (such as about any of 1, 2, or 3) amino acid substitutions; VH-CDR2 comprising the amino acid sequence of SEQ ID NO:57, or a variant thereof comprising up to about 3 (such as about any of 1, 2, or 3) amino acid substitutions; VH-CDR3 comprising the amino acid sequence of SEQ ID NO:49, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:47, or a variant thereof comprising up to about 3 (such as about any of 1, 2, or 3) amino acid substitutions; VH-CDR2 comprising the amino acid sequence of SEQ ID NO:57, or a variant thereof comprising up to about 3 (such as about any of 1, 2, or 3) amino acid substitutions; VH-CDR3 comprising the amino acid sequence of SEQ ID NO:49; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:47; VH-CDR2 comprising the amino acid sequence of SEQ ID NO:57; VH-CDR3 comprising the amino acid sequence of SEQ ID NO:49; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:59, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:59 and a light chain comprising the amino acid sequence of SEQ ID NO:25. In some embodiments, the monoclonal anti-C5 antibody is humanized. In some embodiments the monoclonal anti-C5 antibody is a chimeric antibody.

In some embodiments, the anti-C5 antibody, fusion protein, or an antigen binding fragment thereof comprises a heavy chain comprising the amino acid sequence of SEQ ID NO:76, or a variant thereof. In other embodiments, the anti-C5 antibody comprises a light chain comprising the amino acid sequence of SEQ ID NO:74, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:76 and a light chain comprising the amino acid sequence of SEQ ID NO:74. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:37; VH-CDR2: SEQ ID NO:62; VH-CDR3: SEQ ID NO:39; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:37; VH-CDR2: SEQ ID NO:62; VH-CDR3: SEQ ID NO:39; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR2 comprising the amino acid sequence of SEQ ID NO:62, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:62; and VL-CDR2: SEQ ID NO:9.

In some embodiments, the anti-C5 antibody, fusion protein, or antigen-binding fragment thereof comprises: VH-CDR1 comprising the amino acid sequence of SEQ ID NO:37, or a variant thereof comprising up to about 3 (such as about any of 1, 2, or 3) amino acid substitutions; VH-CDR2 comprising the amino acid sequence of SEQ ID NO:62, or a variant thereof comprising up to about 3 (such as about any of 1, 2, or 3) amino acid substitutions; VH-CDR3 comprising the amino acid sequence of SEQ ID NO:39, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:37, or a variant thereof comprising up to about 3 (such as about any of 1, 2, or 3) amino acid substitutions; VH-CDR2 comprising the amino acid sequence of SEQ ID NO:62, or a variant thereof comprising up to about 3 (such as about any of 1, 2, or 3) amino acid substitutions; VH-CDR3 comprising the amino acid sequence of SEQ ID NO:39; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:37; VH-CDR2 comprising the amino acid sequence of SEQ ID NO:62; VH-CDR3 comprising the amino acid sequence of SEQ ID NO:39; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:64, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:64 and a light chain comprising the amino acid sequence of SEQ ID NO:25. In some embodiments, the monoclonal anti-C5 antibody is humanized. In some embodiments the monoclonal anti-C5 antibody is a chimeric antibody.

In some embodiments, the anti-C5 antibody, fusion protein, or an antigen binding fragment thereof comprises a heavy chain comprising the amino acid sequence of SEQ ID NO:72, or a variant thereof. In other embodiments, the anti-C5 antibody comprises a light chain comprising the amino acid sequence of SEQ ID NO:74, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:72 and a light chain comprising the amino acid sequence of SEQ ID NO:74. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:42; VH-CDR2: SEQ ID NO:65; VH-CDR3: SEQ ID NO:44; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:42; VH-CDR2: SEQ ID NO:65; VH-CDR3: SEQ ID NO:44; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR2 comprising the amino acid sequence of SEQ ID NO:65, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:65; and VL-CDR2: SEQ ID NO:9.

In some embodiments, the anti-C5 antibody, fusion protein, or antigen-binding fragment thereof comprises: VH-CDR1 comprising the amino acid sequence of SEQ ID NO:42, or a variant thereof comprising up to about 3 (such as about any of 1, 2, or 3) amino acid substitutions; VH-CDR2 comprising the amino acid sequence of SEQ ID NO:65, or a variant thereof comprising up to about 3 (such as about any of 1, 2, or 3) amino acid substitutions; VH-CDR3 comprising the amino acid sequence of SEQ ID NO:44, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:42, or a variant thereof comprising up to about 3 (such as about any of 1, 2, or 3) amino acid substitutions; VH-CDR2 comprising the amino acid sequence of SEQ ID NO:65, or a variant thereof comprising up to about 3 (such as about any of 1, 2, or 3) amino acid substitutions; VH-CDR3 comprising the amino acid sequence of SEQ ID NO:44; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:42; VH-CDR2 comprising the amino acid sequence of SEQ ID NO:65; VH-CDR3 comprising the amino acid sequence of SEQ ID NO:44; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:67, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:67 and a light chain comprising the amino acid sequence of SEQ ID NO:25. In some embodiments, the monoclonal anti-C5 antibody is humanized. In some embodiments the monoclonal anti-C5 antibody is a chimeric antibody.

In some embodiments, the anti-C5 antibody, fusion protein, or an antigen binding fragment thereof comprises a heavy chain comprising the amino acid sequence of SEQ ID NO:78, or a variant thereof. In other embodiments, the anti-C5 antibody comprises a light chain comprising the amino acid sequence of SEQ ID NO:74, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:78 and a light chain comprising the amino acid sequence of SEQ ID NO:74. In some embodiments, the monoclonal anti-C5 antibody is humanized. In some embodiments the monoclonal anti-C5 antibody is a chimeric antibody.

In one embodiment, the anti-C5 antibody, fusion protein, or an antigen-binding fragment thereof comprises at least one of the CDRs selected from the group consisting of: VH-CDR1: SEQ ID NO:52; VH-CDR2: SEQ ID NO:68; VH-CDR3: SEQ ID NO:54; VL-CDR1: SEQ ID NO:23; VL-CDR2: SEQ ID NO:9; and VL-CDR3: SEQ ID NO:10, or a variant or variants thereof. In another embodiment, the anti-C5 antibody comprises all of the CDRs of the group consisting of: VH-CDR1: SEQ ID NO:52; VH-CDR2: SEQ ID NO:68; VH-CDR3: SEQ ID NO:54; VL-CDR1: SEQ ID NO:23; VL-CDR2: SEQ ID NO:9; and VL-CDR3: SEQ ID NO:10, or a variant or variants thereof.

In some embodiments, the anti-C5 antibody, fusion protein, or antigen-binding fragment thereof comprises: VH-CDR2 comprising the amino acid sequence of SEQ ID NO:68, or a variant thereof comprising up to about 3 (such as about any of 1, 2, or 3) amino acid substitutions; and VL-CDR2: SEQ ID NO:9, or a variant thereof comprising up to about 3 (such as about any of 1, 2, or 3) amino acid substitutions. In some embodiments, the anti-C5 antibody or an antigen-binding fragment thereof comprises: VH-CDR2 comprising the amino acid sequence of SEQ ID NO:68; and VL-CDR2: SEQ ID NO:9.

In some embodiments, the anti-C5 antibody, fusion protein, or antigen-binding fragment thereof comprises: VH-CDR1 comprising the amino acid sequence of SEQ ID NO:52, or a variant thereof comprising up to about 3 (such as about any of 1, 2, or 3) amino acid substitutions; VH-CDR2 comprising the amino acid sequence of SEQ ID NO:68, or a variant thereof comprising up to about 3 (such as about any of 1, 2, or 3) amino acid substitutions; VH-CDR3 comprising the amino acid sequence of SEQ ID NO:54, or a variant thereof comprising up to about 3 (such as about any of 1, 2, or 3) amino acid substitutions; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10, or a variant thereof comprising up to about 3 (such as about any of 1, 2, or 3) amino acid substitutions.

In some embodiments, the anti-C5 antibody, fusion protein, or antigen-binding fragment thereof comprises: VH-CDR1 comprising the amino acid sequence of SEQ ID NO:52, or a variant thereof comprising up to about 3 (such as about any of 1, 2, or 3) amino acid substitutions; VH-CDR2 comprising the amino acid sequence of SEQ ID NO:68, or a variant thereof comprising up to about 3 (such as about any of 1, 2, or 3) amino acid substitutions; VH-CDR3 comprising the amino acid sequence of SEQ ID NO:54; VL-CDR1 comprising the amino acid sequence of SEQ ID NO:23, or a variant thereof comprising up to about 3 (such as about any of 1, 2, or 3) amino acid substitutions; VL-CDR2 comprising the amino acid sequence of SEQ ID NO:9, or a variant thereof comprising up to about 3 (such as about any of 1, 2, or 3) amino acid substitutions; and VL-CDR3 comprising the amino acid sequence of SEQ ID NO:10.

In some embodiments, the anti-C5 antibody, fusion protein, or antigen-binding fragment thereof comprises: VH-CDR1 comprising the amino acid sequence of SEQ ID NO:52; VH-CDR2 comprising the amino acid sequence of SEQ ID NO:68; VH-CDR3 comprising the amino acid sequence of SEQ ID NO:54; VL-CDR1 comprising the amino acid sequence of SEQ ID NO:23; VL-CDR2 comprising the amino acid sequence of SEQ ID NO:9; and VL-CDR3 comprising the amino acid sequence of SEQ ID NO:10.

In some embodiments, the anti-C5 antibody, fusion protein, or an antigen binding fragment thereof comprises a heavy chain comprising the amino acid sequence of SEQ ID NO:70, or a variant thereof. In other embodiments, the anti-C5 antibody comprises a light chain comprising the amino acid sequence of SEQ ID NO:25, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:70 and a light chain comprising the amino acid sequence of SEQ ID NO:25. In some embodiments, the monoclonal anti-C5 antibody is humanized. In some embodiments the monoclonal anti-C5 antibody is a chimeric antibody.

In some embodiments, the anti-C5 antibody, fusion protein, or an antigen binding fragment thereof comprises a heavy chain comprising the amino acid sequence of SEQ ID NO:80, or a variant thereof. In other embodiments, the anti-C5 antibody comprises a light chain comprising the amino acid sequence of SEQ ID NO:74, or a variant thereof. In another embodiment, the anti-C5 antibody is mAb H1-8/L1-9, or a variant thereof. The monoclonal anti-C5 antibody mAb H1-8/L1-9 comprises a heavy chain comprising the amino acid sequence of SEQ ID NO:78 and a light chain comprising the amino acid sequence of SEQ ID NO:74. In some embodiments, the monoclonal anti-C5 antibody is humanized. In some embodiments the monoclonal anti-C5 antibody is a chimeric antibody.

In some embodiments, the anti-C5 antibody, fusion protein, or antigen-binding fragment thereof comprises a substitution of proline at position #4 (i.e., P4) in VH CDR2, relative to SEQ ID NO:4. In various embodiments, the substitution at P4 is P4→F4 (i.e., P4F), P4→L4 (i.e., P4L), P4→M4 (i.e., P4M), P4→W4 (i.e., P4W), or P4→I4 (i.e., P4I)

In some embodiments, the anti-C5 antibody, fusion protein, or antigen-binding fragment thereof comprises a substitution of threonine at position #9 (i.e., T9) in VH CDR2, relative to SEQ ID NO:4. In various embodiments, the substitution at T9 is T9→H9 (i.e., T9H), T9 is T9→F9 (i.e., T9F), T9→L9 (i.e., T9L), T9→M9 (i.e., T9M), T9→W9 (i.e., T9W), or T9→I9 (i.e., T9I).

In some embodiments, the anti-C5 antibody, fusion protein, or antigen-binding fragment thereof comprises a substitution of proline at position #4 (i.e., P4) in VH CDR2, relative to SEQ ID NO:4; and a substitution of threonine at position #9 (i.e., T9) in VH CDR2, relative to SEQ ID NO:4. In various embodiments, the substitution at P4 is P4→F4 (i.e., P4F), P4→L4 (i.e., P4L), P4→M4 (i.e., P4M), P4→W4 (i.e., P4W), or P4→I4 (i.e., P4I); and the substitution at T9 is T9→H9 (i.e., T9H), T9 is T9→F9 (i.e., T9F), T9→L9 (i.e., T9L), T9→M9 (i.e., T9M), T9→W9 (i.e., T9W), or T9→I9 (i.e., T9I).

In some embodiments, the anti-C5 antibody, fusion protein, or antigen-binding fragment thereof comprises a substitution of valine at position #16 (i.e., V16) in VH CDR3, relative to SEQ ID NO:5. In various embodiments, the substitution at V4 is V16→F16 (i.e., V16F), V16→E16 (i.e., V16E) or V16→W16 (i.e., V16W).

In some embodiments, the anti-C5 antibody, fusion protein, or antigen-binding fragment thereof comprises a substitution of asparagine at position #8 (i.e., N8) in VH CDR1, relative to SEQ ID NO:3. In various embodiments, the substitution at N8 is N8→H8 (i.e., N8H), N8→W8 (i.e., N8W), N8→I8 (i.e., N8I), N8→V8 (i.e., N8V), N8→Y8 (i.e., N8Y), or N8→F8 (i.e., N8F).

In some embodiments, the anti-C5 antibody, fusion protein, or antigen-binding fragment thereof comprises a substitution of leucine at position #9 (i.e., L9) in VH CDR1, relative to SEQ ID NO:20. In various embodiments, the substitution at L9 is L9→W9 (i.e., L9W), L9→I9 (i.e., L9I), L9→V9 (i.e., L9V), L9→Y9 (i.e., L9Y), or L9→F9 (i.e., L9F).

In some embodiments, the anti-C5 antibody, fusion protein, or antigen-binding fragment thereof comprises a substitution of two or more of the group consisting of proline 4 (i.e., P4) in VH CDR2, relative to SEQ ID NO:4, threonine 9 (i.e., T9) in VH CDR2, relative to SEQ ID NO:4, valine 16 (i.e., V16) in VH CDR3, relative to SEQ ID NO:5, and leucine 9 (i.e., L9) in VH CDR1, relative to SEQ ID NO:20. In various embodiments, the anti-C5 antibody or antigen-binding fragment thereof comprising a substitution at two or more of the group consisting of proline 4 (i.e., P4) in VH CDR2, relative to SEQ ID NO:4, threonine 9 (i.e., T9) in VH CDR2, relative to SEQ ID NO:4, valine 16 (i.e., V16) in VH CDR3, relative to SEQ ID NO:5, and leucine 9 (i.e., L9) in VH CDR1, relative to SEQ ID NO:20 comprises the two or more substitutions selected from the group consisting of L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4W/V16E, L9I/P4F/V16E, L9F/P4M/V16E, L9F/P4W/V16E, L9F/P4F/V16E, L9I/P4M/T9H/V16W, L9I/P4W/T9H/V16W, L9I/P4F/T9H/V16W, L9F/P4M/T9H/V16W, L9F/P4W/T9H/V16W, L9F/P4F/T9H/V16W, L9I/P4M/T9H/V16E, L9I/P4W/T9H/V16E, L9I/P4F/T9H/V16E, L9F/P4M/T9H/V16E, L9F/P4W/T9H/V16E, and L9F/P4F/T9H/V16E.

In some embodiments, the antibodies or fusion proteins are chimeric antibodies. In some embodiments, the anti-human C5 antibody may comprise human light chain and human heavy chain constant regions in combination with the variable region CDR sequences described in the specification above. One of skill in the art would be able to prepare and obtain a chimeric antibody using known techniques of swapping relevant domains of specific antibodies of interest. Such an antibody is easily prepared by grafting heterogeneous antibody domains, incorporating one or more CDR sequences described in this application. Using known recombinant technology, it is possible to obtain and prepare a recombinant antibody comprising heavy and light chain constant regions encoded by nucleic acid sequences of human heavy and light chain constant regions; and the heavy and light chain variable regions comprising CDRs encoded by nucleic acid sequences corresponding to the CDR sequences set forth in the disclosure. One of skill in the art can prepare an anti-human C5 antibody comprises one or more CDR sequences described in this disclosure, wherein portions of the light chain alone or portions of the heavy chain alone are replaced with regions from an antibody belonging to another species, such as, for example, human. A human anti-human-C5 antibody comprising variable regions having one or more CDR sequences selected from SEQ ID NOs: 3-5, 8-11, 14, 17, 20, 23, 26, 29, 34, 37-39, 42-44, 47-49, 52-54, 57, 62, 65, and 68 or a variant or variants thereof, in combination with murine or non-murine antibody structural elements outside the CDR regions can be prepared by routine methods known in the art. In some embodiments, the antibodies or antibody fragments are further humanized using known techniques in the art.

In various embodiments, any of the antibodies or fusion proteins of the invention described herein, having any of the variable regions described herein, may comprise a human IgG4 constant heavy chain. SEQ ID NO:32 is an example amino acid sequence of a human IgG4 constant heavy chain. In some embodiments, the antibody or fusion protein of the invention comprises a human IgG4 constant heavy chain having an S228P mutation. SEQ ID NO:33 is an example amino acid sequence of a human IgG4 constant heavy chain having an S228P mutation. In some embodiments, the antibody of the invention comprises a human IgG4 constant heavy chain having an Fc PLA mutation. SEQ ID NO:61 is an example amino acid sequence of a human IgG4 constant heavy chain having an Fc PLA mutation.

In some embodiments the anti-C5 antibody or fusion protein comprises an antibody having at least about 85% (such as at least about any of 85%, 86%, 87%, 88%, 89%, 90%, 91%, 92%, 93%, 94%, 95%, 96%, 97%, 98%, 99%, or 100%) amino acid identity with the CDR sequence described herein, listed in SEQ ID NOs 3-5, 8-11, 14, 17, 20, 23, 26, 29, 34, 37-39, 42-44, 47-49, 52-54, 57, 62, 65, and 68.

In one embodiment, the current disclosure encompasses an anti-C5 antibody or fusion protein having CDR sequences of at least about 85% (such as at least about any of 85%, 86%, 87%, 88%, 89%, 90%, 91%, 92%, 93%, 94%, 95%, 96%, 97%, 98%, 99%, or 100%) identity to the CDR sequences described above. In one embodiment, the antibody against human C5 has a VH region and a VL region, wherein the VH region has an amino acid sequence that is more than about 90% (such as more than about any of 90%, 91%, 92%, 93%, 94%, 95%, 96%, 97%, 98%, 99%, or 100%) identical to one selected from SEQ ID NOs 2, 19, 22, 28, 36, 41, 46, 51, 56, and 59, and wherein the VL region has an amino acid sequence that is more than about 90% (such as more than about any of 90%, 91%, 92%, 93%, 94%, 95%, 96%, 97%, 98%, 99%, or 100%) identical to one selected from SEQ ID NOs 7, 13, 16, 17, 25, 31, and 74.

In some embodiments the antibody or the antibody fragment is modified. In some embodiments the modifications include fusion of the antibody or the antigen-binding fragment thereof with portions of another protein, or a protein fragment. In some embodiments the antibody or the antibody fragment thereof of the invention is modified to increase the circulating half-life of the same in vivo. For example, the antibody of the fragment may be fused with an FcRn molecule, which is also known as neonatal Fc receptor to stabilize the antibody in vivo. (Nature Reviews Immunology 7:715-725). In some embodiments, the antibody or antigen-binding fragment thereof is conjugated (e.g., fused) to an effector molecule and/or another targeting moiety (such as an antibody or antibody fragment recognizing a different molecule, different antigen or a different epitope).

One of skill in the art would be able to prepare human-C5 binding scFv, comprising at least one specific CDR sequence selected from SEQ ID NOs 3-5, 8-11, 14, 17, 20, 23, 26, 29, 34, 37-39, 42-44, 47-49, 52-54, 57, 62, 65, and 68 or a variant or variants thereof. An scFv may comprise heavy chain variable region sequences designated in SEQ ID NOs 3-5, 17, 20, 26, 34, 37-39, 42-44, 47-49, 52-54, 57, 62, 65, and 68, or a variant or variants thereof, and light chain variable regions designated in SEQ ID NOs 8-11, 14, 23, and 29, or a variant or variants thereof. CDR sequences incorporated within the scFv having amino acid sequence identity of at least about 85% 86%, 87%, 88%, 89%, 90%, 91%, 92%, 93%, 94%, 95%, 96%, 97%, 98%, or 99% to the CDR sequences described in the present disclosure are encompassed within the scope of the present disclosure.

In some embodiments, the isolated antibody binds to human C5, wherein the antibody binds to an epitope of human C5. In some embodiments, the human C5 antibody of the invention is one that binds to a specific epitope of human C5. In some embodiments, the epitope includes at least one amino acid in the α-chain of C5. In some embodiments, the epitope includes at least one amino acid in the I3-chain of C5.

In some embodiments, the present invention provides a fusion protein comprising an anti-C5 antibody moiety (such as any one of the anti-C5 antibodies described herein) and an FH or a functional fragment thereof. In some embodiments, the anti-C5 antibody moiety is a full-length antibody. In some embodiments, the FH is fused to the one or both the heavy chain of the full-length antibody (for example at the C-terminal end of the heavy chain(s)). In some embodiments, the full-length antibody comprises an IgG4 chain (such as an IgG4 chain comprising an PLA mutation). For example, in one embodiment, the IgG4 chain comprising an PLA mutation comprises a human IgG4 constant heavy chain comprising an Fc PLA mutation. In one embodiment, the IgG4 chain comprising an PLA mutation comprises an amino acid sequence set forth in SEQ ID NO:61.

In some embodiments, the present invention provides a fusion protein comprising an anti-C5 antibody moiety (such as any one of the anti-C5 antibodies described herein) and an FH or a functional fragment thereof comprising SCR1-5 domains of FH. In some embodiments, the present invention provides a fusion protein comprising an anti-C5 antibody moiety (such as any one of the anti-C5 antibodies described herein) comprising an IgG4 chain (e.g., an IgG4 chain comprising a PLA mutation) and an FH or a functional fragment thereof comprising SCR1-5 domains of FH.

In some embodiments, the present invention provides a fusion protein comprising an anti-C5 antibody moiety and an FH or a functional fragment thereof, wherein the anti-C5 antibody moiety is pH sensitive (such as any one of the pH sensitive anti-C5 antibodies described herein). For example, in some embodiments, the pH sensitive anti-C5 antibody moiety is an anti-C5 antibody moiety that binds to C5 at a higher affinity at pH 7.4 than at pH 5.8. In some embodiments, the binding affinity of the anti-C5 antibody moiety to C5 (e.g., human C5) at pH 7.4 is at least about 3 times (such as at least about any of 4, 5, 6, 7, 8, or 10) times higher than the binding affinity of the anti-C5 antibody moiety to C5 (e.g., human C5) at pH 5.8.

In some embodiments, the fusion protein comprises an anti-C5 antibody moiety and an FH or a functional fragment thereof, wherein the anti-C5 antibody moiety is pH sensitive (such as any one of the pH sensitive anti-C5 antibodies described herein), for example binding to C5 at a higher affinity at pH 7.4 than at pH 5.8, wherein the anti-C5 moiety comprising an IgG4 chain (e.g., an IgG4 chain comprising a PLA mutation). In some embodiments, the fusion protein comprises an anti-C5 antibody moiety and an FH or a functional fragment thereof, wherein the anti-C5 antibody moiety is pH sensitive (such as any one of the pH sensitive anti-C5 antibodies described herein), for example binding to C5 at a higher affinity at pH 7.4 than at pH 5.8, and wherein the FH or a functional fragment thereof comprises SCR1-5 domains of FH. In some embodiments, the fusion protein comprises an anti-C5 antibody moiety and an FH or a functional fragment thereof, wherein the anti-C5 antibody moiety is pH sensitive (such as any one of the pH sensitive anti-C5 antibodies described herein), for example binding to C5 at a higher affinity at pH 7.4 than at pH 5.8, wherein the anti-C5 moiety comprises an IgG4 chain (e.g., an IgG4 chain comprising a PLA mutation), and wherein the FH or a functional fragment thereof comprises SCR1-5 domains of FH.

In some embodiments, the present invention provides a fusion protein comprising an anti-C5 antibody moiety (such as any one of the anti-C5 antibodies described herein) and an FH or a functional fragment thereof, wherein the anti-C5 moiety comprises a histidine substitution in one or more of its CDR regions (such as any one of the histidine-containing anti-C5 antibodies described herein). In some embodiments, the fusion protein comprises an anti-C5 antibody moiety and an FH or a functional fragment thereof, wherein the anti-C5 moiety comprises a histidine substitution in one or more of its CDR regions (such as any one of the histidine-containing anti-C5 antibodies described herein), and wherein the anti-C5 moiety comprises an IgG4 chain (e.g., an IgG4 chain comprising a PLA mutation). In some embodiments, the fusion protein comprises an anti-C5 antibody moiety and an FH or a functional fragment thereof, wherein the anti-C5 moiety comprises a histidine substitution in one or more of its CDR regions (such as any one of the histidine-containing anti-C5 antibodies described herein), and wherein the FH or a functional fragment thereof comprises SCR1-5 domains of FH. In some embodiments, the fusion protein comprises an anti-C5 antibody moiety and an FH or a functional fragment thereof, wherein the anti-C5 moiety comprises a histidine substitution in one or more of its CDR regions (such as any one of the histidine-containing anti-C5 antibodies described herein), wherein the anti-C5 moiety comprises an IgG4 chain (e.g., an IgG4 chain comprising a PLA mutation), and wherein the FH or a functional fragment thereof comprises SCR1-5 domains of FH.

In various embodiments, the FH or functional fragment thereof comprises a complement FH. Complement FH is a single polypeptide chain plasma glycoprotein. The protein is composed of 20 repetitive SCR domains of approximately 60 amino acids, arranged in a continuous fashion like a string of 20 beads. In some embodiments, the FH binds to C3b, accelerates the decay of the alternative pathway C3-convertase (C3Bb), and acts as a cofactor for the proteolytic inactivation of C3b. In the presence of FH, C3b proteolysis results in the cleavage of C3b. In some embodiments, the FH has at least three distinct binding domains for C3b, which are located within SCR 1-4, SCR 5-8, and SCR 19-20. In some embodiments, each site of FH binds to a distinct region within the C3b protein: the N-terminal sites bind to native C3b; the second site, located in the middle region of FH, binds to the C3c fragment and the sited located within SCR19 and 20 binds to the C3d region. In some embodiments, the FH additionally contains binding sites for heparin, which are located within SCR 7, SCR 5-12, and SCR20 of FH and overlap with that of the C3b binding site. For example, in some embodiments, structural and functional analyses have shown that the domains for the complement inhibitory activity of FH are located within the first four N-terminal SCR domains.

The FH or functional fragment thereof described herein refers to any portion of a FH protein having some or all the complement inhibitory activity of the FH protein, and includes, but is not limited to, full-length FH proteins, biologically active fragments of FH proteins, a FH fragment comprising SCR1-4, or any homologue of a naturally occurring FH or fragment thereof, as described in detail below.

In various embodiments, the FH is a full-length human FH protein comprises an amino acid sequence set forth in SEQ ID NO:81, SEQ ID NO:82, SEQ ID NO:83, or SEQ ID NO:84. Amino acids 1-18 correspond to the leader peptide, amino acids 21-80 correspond to SCR1, amino acids 85-141 correspond to SCR2, amino acids 146-205 correspond to SCR3, amino acids 210-262 correspond to SCR4, amino acids 267-320 correspond to SCR5.

In some embodiments, the FH portion has one or more of the following properties: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

In some embodiments, the FH portion comprises the first four N-terminal SCR domains of FH (SCR 1-4). In some embodiments, the construct comprises the first five N-terminal SCR domains of FH (SCR 1-5). In some embodiments, the construct comprises the first six N-terminal SCR domains of FH (SCR 1-6). In some embodiments, the FH portion comprises (and in some embodiments consists of or consisting essentially of) at least the first four N-terminal SCR domains of FH, including for example, at least any of the first 5, 6, 7, 8, 9, 10, 11, 12, 13, 14, or more N-terminal SCR domains of FH.

In some embodiments, the FH is a wildtype FH. In some embodiments, the FH is a variant of a naturally occurring FH or a variant thereof. A variant of a naturally occurring FH protein or a fragment thereof includes proteins which differ from a naturally occurring FH or a fragment thereof in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a FH variant may have an amino acid sequence that is at least about 70% identical to the amino acid sequence of a naturally occurring FH (e.g., SEQ ID NO:81, SEQ ID NO:82, SEQ ID NO:83, or SEQ ID NO:84), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring FH (e.g., SEQ ID NO:81, SEQ ID NO:82, SEQ ID NO:83, or SEQ ID NO:84). In some embodiment, the FH variant or a fragment thereof retains all the complement inhibition activity of a naturally occurring FH or a fragment thereof. In some embodiments, the FH variant or a fragment thereof retains at least about 50%, for example, at least about any of 60%, 70%, 80%, 90%, or 95% of the complement inhibition activity of a naturally occurring FH or a fragment thereof. In some embodiments, the FH or fragment thereof comprises at least the first five N-terminal SCR domains of a human FH, such as a FH portion having an amino acid sequence containing at least amino acids 21 through 320 of the human FH (e.g., SEQ ID NO:81, SEQ ID NO:82, SEQ ID NO:83, or SEQ ID NO:84). In some embodiments, the FH or fragment thereof comprises at least the first five N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 320 of the human FH (e.g., SEQ ID NO:81, SEQ ID NO:82, SEQ ID NO:83, or SEQ ID NO:84).

### Screening Assays

The present invention has application in various screening assays, including, determining whether a candidate anti-C5 antibody (e.g., anti-C5 antibody, anti-C5 fusion protein antibody, etc.) can inhibit complement activity.

In some embodiments, the level of complement activity in the presence of the candidate anti-C5 antibody is compared with complement activity detected in a positive comparator control. The positive comparator control comprises complement activation in the absence of added test compound. In some embodiments, the candidate anti-C5 antibody is identified as an inhibitor of the complement when the complement activity in the presence of the candidate anti-C5 antibody is less than about 70% of the complement activity detected in a positive comparator control; this corresponds to greater than about 30% inhibition of complement activity in the presence of the test compound. In other embodiments, the candidate anti-C5 antibody is identified as an inhibitor of the complement when the complement activity in the presence of the candidate anti-C5 antibody is less than about 80% of the complement activity detected in a positive comparator control; this corresponds to greater than about 20% inhibition of complement activity in the presence of the test compound. In still other embodiments, the candidate anti-C5 antibody is identified as an inhibitor of the complement when the complement activity in the presence of the candidate anti-C5 antibody is less than about 90% of the complement activity detected in a positive comparator control; this corresponds to greater than about 10% inhibition of complement activity in the presence of the test compound. In some embodiments, the level of complement inhibition by the candidate anti-C5 antibody is compared with the level of inhibition detected in a negative comparator control.

A variety of immunoassay formats, including competitive and non-competitive immunoassay formats, antigen capture assays, two-antibody sandwich assays, and three-antibody sandwich assays are useful methods of the invention (Self et al., 1996, Curr. Opin. Biotechnol. 7:60-65). The invention should not be construed to be limited to any one type of known or hereto for unknown assay, provided that the assay is able to detect the inhibition of complement.

Enzyme-linked immunosorbent assays (ELISAs) are useful in the methods of the invention. An enzyme such as, but not limited to, horseradish peroxidase (HRP), alkaline phosphatase, beta-galactosidase or urease can be linked, for example, to an anti-C5 antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, Mo.). Useful enzyme-linked primary and secondary antibodies can be obtained from any number of commercial sources.

Chemiluminescent detection is also useful for detecting the inhibition of the terminal complement pathway. Chemiluminescent secondary antibodies may be obtained from any number of commercial sources.

Fluorescent detection is also useful for detecting the inhibition of the terminal complement. Useful fluorochromes include, but are not limited to, DAPI, fluorescein, Hoechst 33258, R-phycocyanin, B-phycoerythrin, R-phycoerythrin, rhodamine, Texas red and lissamine- Fluorescein- or rhodamine-labeled antibodies.

Radioimmunoassays (RIAs) are also useful in the methods of the invention. Such assays are well known in the art, and are described for example in Brophy et al. (1990, Biochem. Biophys. Res. Comm. 167:898-903) and Guechot et al. (1996, Clin. Chem. 42:558-563). Radioimmunoassays are performed, for example, using Iodine-125-labeled primary or secondary antibody (Harlow et al., supra, 1999).

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis is performed using a spectrophotometer. It is understood that the assays of the invention can be performed manually or, if desired, can be automated and that the signal emitted from multiple samples can be detected simultaneously in many systems available commercially.

The methods of the invention also encompass the use of capillary electrophoresis based immunoassays (CEIA), which can be automated, if desired Immunoassays also may be used in conjunction with laser-induced fluorescence as described, for example, in Schmalzing et al. (1997, Electrophoresis 18:2184-2193) and Bao (1997, J. Chromatogr. B. Biomed. Sci. 699:463-480). Liposome immunoassays, such as flow-injection liposome immunoassays and liposome immunosensors, may also be used according to the methods of the invention (Rongen et al., 1997, J. Immunol. Methods 204:105-133).

Quantitative western blotting may also be used to determine the level of terminal complement inhibition in the methods of the invention. Western blots are quantified using well known methods such as scanning densitometry (Parra et al., 1998, J. Vasc. Surg. 28:669-675).

### Methods of Administration

The methods of the invention comprise administering a therapeutically effective amount of at least one anti-C5 antibody (e.g., anti-C5 antibody, anti-C5 fusion protein antibody, etc.), or binding fragment thereof (such as any of the antibodies or fragments thereof described elsewhere herein), to an individual identified as having a complement-mediated disease or disorder. In one embodiment the individual is a mammal having a complement system. In one embodiment the individual is a human. In various embodiments, the at least one anti-C5 antibody, or binding fragment thereof, is administered locally, regionally, or systemically.

In various embodiments, the disease or disorder is at least selected from the group consisting of: MD, AMD, ischemia reperfusion injury, arthritis, rheumatoid arthritis, lupus, ulcerative colitis, stroke, post-surgery systemic inflammatory syndrome, asthma, allergic asthma, COPD, PNH syndrome, myasthenia gravis, NMO, multiple sclerosis, delayed graft function, antibody-mediated rejection, aHUS, CRVO, CRAO, epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, ANCA-mediated glomerulonephritis, lupus nephritis, and combinations thereof), ANCA-mediated vasculitis, Shiga toxin induced HUS, and antiphospholipid antibody-induced pregnancy loss, or any combinations thereof. In some embodiments, the complement-mediated disease is C3 glomerulopathy. In some embodiments, the complement-mediated disease is macular degeneration, such as age-related macular degeneration. In one embodiment, administration of the anti-C5 antibody fusion protein inhibits the generation of a C3a or C3b protein. In one embodiment, administration of the anti-C5 antibody fusion protein inhibits the generation of a C5a or C5b protein. In some embodiments, the compositions and methods of the invention are useful for treating subject, including subjects having PNH, who are not responsive to treatment with eculizumab. By way of non-limiting example, some subjects may have a mutation in the alpha chain of C5 that may render them resistant to treatment of eculizumab (see Genetic variants in C5 and poor response to eculizumab (Nishimura J, et al., N Engl J Med. 2014 Feb. 13; 370(7):632-9).

The methods of the invention can comprise the administration of at least one anti-C5 antibody fusion protein, or binding fragment thereof, but the present invention should in no way be construed to be limited to the anti-C5 antibody fusion proteins described herein, but rather should be construed to encompass any anti-C5 antibody fusion protein, both known and unknown, that diminish and reduce complement activation.

The method of the invention comprises administering a therapeutically effective amount of at least one anti-C5 antibody fusion protein, or binding fragment thereof, to an individual wherein a composition of the present invention comprising at least one anti-C5 antibody fusion protein, or binding fragment thereof, either alone or in combination with at least one other therapeutic agent. The invention can be used in combination with other treatment modalities, such as, for example anti-inflammatory therapies, and the like. Examples of anti-inflammatory therapies that can be used in combination with the methods of the invention include, for example, therapies that employ steroidal drugs, as well as therapies that employ non-steroidal drugs.

The method of the invention comprises administering a therapeutically effective amount of an anti-C5 antibody fusion protein, or an antigen-binding fragment thereof, to a subject. In some embodiments, the invention encompasses a method of treatment of complement-related diseases involving dysregulation of the complement signaling by administering a therapeutically effective amount of an antibody fusion protein of the invention, or a therapeutically effective amount of an antibody fragment thereof, such that a reduction of C3a, C3b, C5a, or C5b or MAC formation is effected in the subject. In some embodiments the invention encompasses a method of treatment of complement-related diseases involving dysregulation of complement signaling by administering a therapeutically effective amount of an antibody fusion protein or an antibody fragment. In some embodiments the invention encompasses a method of treatment of complement-related diseases involving dysregulation of complement signaling by administering to a subject an effective amount of an antibody fusion protein, an antibody fragment, a polypeptide, a peptide, a conjugated peptide, such that the complement activation pathway activation is reduced in the subject. In some embodiments, the method of treatment encompasses administering to a subject a systemically effective dose of an antibody fusion protein or an antibody fragment, whereby systemic reduction of C3a, C3b, C5a, or C5b or MAC formation is effected in the subject.

The pharmaceutical compositions useful for practicing the invention may be administered to deliver a dose of at least about 1 ng/kg, at least about 5 ng/kg, at least about 10 ng/kg, at least about 25 ng/kg, at least about 50 ng/kg, at least about 100 ng/kg, at least about 500 ng/kg, at least about 1 μg/kg, at least about 5 μg/kg, at least about 10 μg/kg, at least about 25 μg/kg, at least about 50 μg/kg, at least about 100 μg/kg, at least about 500 μg/kg, at least about 1 mg/kg, at least about 5 mg/kg, at least about 10 mg/kg, at least about 25 mg/kg, at least about 50 mg/kg, at least about 100 mg/kg, at least about 200 mg/kg, at least about 300 mg/kg, at least about 400 mg/kg, and at least about 500 mg/kg of body weight of the subject. In one embodiment, the invention administers a dose which results in a concentration of the anti-C5 antibody fusion protein of the present invention of at least about 1 pM, at least about 10 pM, at least about 100 pM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in an individual. In another embodiment, the invention envisions administration of a dose which results in a concentration of the anti-C5 antibody fusion protein of the present invention between at least about 1 pM, at least about 10 pM, at least about 100 pM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in the plasma of an individual.

In some embodiments, the pharmaceutical compositions useful for practicing the invention may be administered to deliver a dose of no more than about 1 ng/kg, no more than about 5 ng/kg, no more than about 10 ng/kg, no more than about 25 ng/kg, no more than about 50 ng/kg, no more than about 100 ng/kg, no more than about 500 ng/kg, no more than about 1 μg/kg, no more than about 5 μg/kg, no more than about 10 μg/kg, no more than about 25 μg/kg, no more than about 50 μg/kg, no more than about 100 μg/kg, no more than about 500 μg/kg, no more than about 1 mg/kg, no more than about 5 mg/kg, no more than about 10 mg/kg, no more than about 25 mg/kg, no more than about 50 mg/kg, no more than about 100 mg/kg, no more than about 200 mg/kg, no more than about 300 mg/kg, no more than about 400 mg/kg, and no more than about 500 mg/kg of body weight of the subject. In one embodiment, the invention administers a dose which results in a concentration of the anti-C5 antibody fusion protein of the present invention of no more than about 1 pM, no more than about 10 pM, no more than about 100 pM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in an individual. In another embodiment, the invention envisions administration of a dose which results in a concentration of the anti-C5 antibody fusion protein of the present invention between no more than about 1 pM, no more than about 10 pM, no more than about 100 pM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in the plasma of an individual. Also contemplated are dosage ranges between any of the doses disclosed herein.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

The antibody fusion protein may be administered to a subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the subject, etc. The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to subjects of all sorts. Modification of pharmaceutical compositions suitable for administration to humans in order to render the compositions suitable for administration to various subjects is well understood, and the ordinarily skilled veterinary pharmacologist can design and perform such modification with merely ordinary, if any, experimentation. Individuals to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intraocular, intravitreal, intramuscular, intradermal and intravenous routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In some embodiments, the droplets provided by this route of administration have an average diameter in the range from about 0.1 to about 200 nanometers.

The formulations are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more additional ingredients.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Cells Producing Antibodies, Fusion Proteins, or Antigen Binding Fragments Thereof

In some embodiments, the invention is a cell or cell line (such as host cells) that produces at least one of the anti-C5 antibodies (e.g., anti-C5 antibody, anti-C5 fusion protein antibody, etc.), or antigen binding fragments, described herein. In one embodiment, the cell or cell line is a genetically modified cell that produces at least one of the anti-C5 antibodies, or antigen binding fragments, described herein. In one embodiment, the cell or cell line is a hybridoma that produces at least one of the anti-C5 antibodies, or antigen binding fragments, described herein.

Hybrid cells (hybridomas) are generally produced from mass fusions between murine splenocytes, which are highly enriched for B-lymphocytes, and myeloma “fusion partner cells” (Alberts et al., Molecular Biology of the Cell (Garland Publishing, Inc. 1994); Harlow et al., Antibodies. A Laboratory Manual (Cold Spring Harbor Laboratory, Cold Spring Harbor, 1988). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are referred to as monoclonal antibodies.

Also provided are nucleic acids encoding any of the antibodies, or antibody fragments, disclosed herein, as well as vectors comprising the nucleic acids. Thus, the antibodies and fragments of the invention can be generated by expressing the nucleic acid in a cell or a cell line, such as the cell lines typically used for expression of recombinant or humanized immunoglobulins. Thus, the antibodies and fragments of the invention can also be generated by cloning the nucleic acids into one or more expression vectors, and transforming the vector into a cell line such as the cell lines typically used for expression of recombinant or humanized immunoglobulins.

The genes encoding the heavy and light chains of immunoglobulins, or fragments thereof, can be engineered according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains, or fragments thereof, can be cloned from an antibody secreting cell's genomic DNA, or cDNA is produced by reverse transcription of the cell's RNA. Cloning is accomplished by conventional techniques including the use of PCR primers that hybridize to the sequences flanking or overlapping the genes, or segments of genes, to be cloned.

Nucleic acids encoding the antibody of the invention, or the heavy chain or light chain or fragments thereof, can be obtained and used in accordance with recombinant nucleic acid techniques for the production of the specific immunoglobulin, immunoglobulin chain, or a fragment or variant thereof, in a variety of host cells or in an in vitro translation system. For example, the antibody-encoding nucleic acids, or fragments thereof, can be placed into suitable prokaryotic or eukaryotic vectors, e.g., expression vectors, and introduced into a suitable host cell by an appropriate method, e.g., transformation, transfection, electroporation, infection, such that the nucleic acid is operably linked to one or more expression control elements, e.g., in the vector or integrated into the host cell genome.

In some embodiments, the heavy and light chains, or fragments thereof, can be assembled in two different expression vectors that can be used to co-transfect a recipient cell. In some embodiments, each vector can contain two or more selectable genes, one for selection in a bacterial system and one for selection in a eukaryotic system. These vectors allow for the production and amplification of the genes in a bacterial system, and subsequent co-transfection of eukaryotic cells and selection of the co-transfected cells. The selection procedure can be used to select for the expression of antibody nucleic acids introduced on two different DNA vectors into a eukaryotic cell.

Alternatively, the nucleic acids encoding the heavy and light chains, or fragments thereof, may be expressed from one vector. Although the light and heavy chains are coded for by separate genes, they can be joined, using recombinant methods. For example, the two polypeptides can be joined by a synthetic linker that enables them to be made as a single protein chain in which the VL and VH regions pair to form monovalent molecules (known as scFv; see e.g., Bird et al., 1988, Science 242: 423-426; and Huston et al., 1988, Proc. Natl. Acad. Sci. USA 85:5879-5883).

The invention provides for an isolated nucleic acid molecule comprising a nucleic acid sequence encoding a heavy chain and/or a light chain, as well as fragments thereof. A nucleic acid molecule comprising sequences encoding both the light and heavy chain, or fragments thereof, can be engineered to contain a synthetic signal sequence for secretion of the antibody, or fragment, when produced in a cell. Furthermore, the nucleic acid molecule can contain specific DNA links which allow for the insertion of other antibody sequences and maintain the translational reading frame so to not alter the amino acids normally found in antibody sequences.

In accordance with the present invention, antibody-encoding nucleic acid sequences can be inserted into an appropriate expression vector. In various embodiments, the expression vector comprises the necessary elements for transcription and translation of the inserted antibody-encoding nucleic acid so as to generate recombinant DNA molecules that direct the expression of antibody sequences for the formation of an antibody, or a fragment thereof.

The antibody-encoding nucleic acids, or fragments thereof, can be subjected to various recombinant nucleic acid techniques known to those skilled in the art such as site-directed mutagenesis.

A variety of methods can be used to express nucleic acids in a cell. Nucleic acids can be cloned into a number of types of vectors. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a murine stem cell virus (MSCV) vector is used to express a desired nucleic acid. MSCV vectors have been demonstrated to efficiently express desired nucleic acids in cells. However, the invention should not be limited to only using a MSCV vector, rather any retroviral expression method is included in the invention. Other examples of viral vectors are those based upon Moloney Murine Leukemia Virus (MoMuLV) and human immunodeficiency virus (HIV). In some embodiments, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193).

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof Δn example of a promoter is the immediate early cytomegalovirus (CMV) promoter sequence.

This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), HIV long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation and the like. Methods for producing cells comprising vectors and/or exogenous nucleic acids are well-known in the art. See, for example, Sambrook et al. (2012) and Ausubel et al. (1999).

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Human C5 Expressing Non-Human Animals

The invention also includes a genetically modified non-human animal that expresses human C5. In some embodiments, the genetically modified non-human animal that expresses human C5 also expresses non-human animal C5. In some embodiments, the genetically modified non-human animal that expresses human C5 does not express non-human animal C5. In one embodiment, the invention is a genetically modified non-human animal that expresses human C5 from the non-human animal's endogenous regulatory elements, but does not express non-human animal C5. In some embodiments, the non-human animal is a mammal. In some embodiments, the non-human animal is a rodent. In some embodiments, the non-human animal is a rat or a mouse. In some embodiments, the mouse is an immunodeficient mouse. In some embodiments, the mouse is NOD/SCID mouse. In some embodiments, the mouse is FcRn/SCID mouse.

To create a genetically modified non-human animal, a nucleic acid encoding the human C5 protein can be incorporated into a recombinant expression vector in a form suitable for expression of the human C5 protein in a host cell. The term “in a form suitable for expression of the fusion protein in a host cell” is intended to mean that the recombinant expression vector includes one or more regulatory sequences operatively linked to the nucleic acid encoding the human C5 protein in a manner which allows for integration into the nonhuman animal genome to result in stable and permanent transcription of the nucleic acid into mRNA and translation of the mRNA into the human C5 protein. The term “regulatory sequence” is art-recognized and intended to include promoters, enhancers and other expression control elements (e.g., polyadenylation signals, PiggyBac and Sleeping Beauty transposon elements). Such regulatory sequences are known to those skilled in the art and are described in 1990, Goeddel, Gene Expression Technology: Methods in Enzymology 185, Academic Press, San Diego, Calif. and in Nakanishi H, Higuchi Y, Kawakami S, Yamashita F, Hashida M. Mol Ther. 2010 April; 18(4):707-14. doi: 10.1038/mt.2009.302. Epub 2010 Jan. 26.; Hudecek M, Ivics Z. Curr Opin Genet Dev. 2018 Jun. 22; 52:100-108. doi: 10.1016/j.gde.2018.06.003. [Epub ahead of print] Review. It should be understood that the design of the expression vector may depend on such factors as the choice of the host cell and animals to be transfected and/or the amount of human C5 protein to be expressed.

A genetically modified non-human animal can be created, for example, by introducing a nucleic acid encoding the human C5 protein (typically linked to appropriate regulatory elements, such as a constitutive or tissue-specific enhancer) into oocyte, e.g., by microinjection, and allowing the oocyte to develop in a female founder mouse. Such animals can also be generated by introducing a nucleic acid encoding the human C5 protein (typically linked to appropriate regulatory elements, such as a constitutive or tissue-specific enhancer and/or PiggyBac and Sleeping Beauty transposon elements) into the animals through hydrodynamic injection via tail vein as described in Suda T, Liu D. Mol Ther. 2007 December; 15(12):2063-9. Epub 2007 Oct. 2. Review. Intronic sequences and polyadenylation signals can also be included in the transgene to increase the efficiency of expression of the transgene. Methods for generating genetically modified animals, such as mice, have become conventional in the art and are described, for example, in U.S. Pat. Nos. 4,736,866 and 4,870,009 and 1986, Hogan et al., A Laboratory Manual, Cold Spring Harbor, N.Y., Cold Spring Harbor Laboratory. A genetically modified founder animal can be used to breed additional subjects carrying the transgene if the transgene is introduced into oocytes. Genetically modified animals carrying a transgene encoding the C5 protein generated via oocyte injection of the invention can further be bred to other genetically modified animals carrying other transgenes, or to other knockout animals, e.g., a knockout mouse that does not express the murine C5 gene. Genetically modified animals carrying a transgene encoding the C5 protein generated via hydrodynamic tail vein injection can be readily produced using other gene knockout or transgenic mice, e.g., FcRn/SCID mice for experimental use. It will be understood that in addition to genetically modified animals, the system can be used to generate other human C5 expressing subjects.

In one embodiment, a genetically modified non-human animal that expresses human C5 from the non-human animal's regulatory elements is generated using a system that replaces the non-human animal's C5 exon sequences (or exon and intron sequences) with human C5 exon sequences (or exon and intron sequences), but leaves one, more, or all of the native non-human animal's regulatory elements (e.g., promoter, enhancers, flanking regions, introns, etc.) sequences unchanged. Although any suitable system can be used, one exemplary system capable of producing a genetically modified non-human animal in this way is the CRISPr/Cas9 system. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III subtypes. Wild-type type II CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39): 15644-9; Sampson et al., Nature. 2013 May 9; 497(7448):254-7; and Jinek, et al., Science. 2012 Aug. 17; 337(6096):816-21

In one embodiment, the genetically modified non-human animal of the invention expresses human C5 from endogenous promoter. Examples of promoters useful in the invention include, but are not limited to, the native mouse promoter, DNA pol II promoter, PGK promoter, ubiquitin promoter, albumin promoter, globin promoter, ovalbumin promoter, SV40 early promoter, the Rous sarcoma virus (RSV) promoter, β-actin promoter, retroviral LTR, and lentiviral LTR. Promoter and enhancer expression systems useful in the invention also include inducible and/or tissue-specific expression systems.

In some embodiments, the genetically modified non-human animal of the invention that expresses human C5 is used for screening, testing, assessing, or evaluating anti-C5 antibodies and anti-C5 mAb fusion proteins. In some embodiments, the genetically modified non-human animal of the invention that expresses human C5 is used for screening, testing, assessing, or evaluating the characteristics, properties or activities of anti-C5 antibodies and anti-C5 mAb fusion proteins.

### Kits

The invention also includes a kit comprising an anti-C5 antibody (e.g., anti-C5 antibody, anti-C5 fusion protein antibody, etc.), or combinations thereof, of the invention and an instructional material which describes, for instance, administering the anti-C5 antibody, or combinations thereof, to an individual as a therapeutic treatment or a non-treatment use as described elsewhere herein. In an embodiment, this kit further comprises a (optionally sterile) pharmaceutically acceptable carrier suitable for dissolving or suspending the therapeutic composition, comprising an anti-C5 antibody, or combinations thereof, of the invention, for instance, prior to administering the antibody to an individual. Optionally, the kit comprises an applicator for administering the antibody.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore and are not to be construed as limiting in any way the remainder of the disclosure.

### Example 1

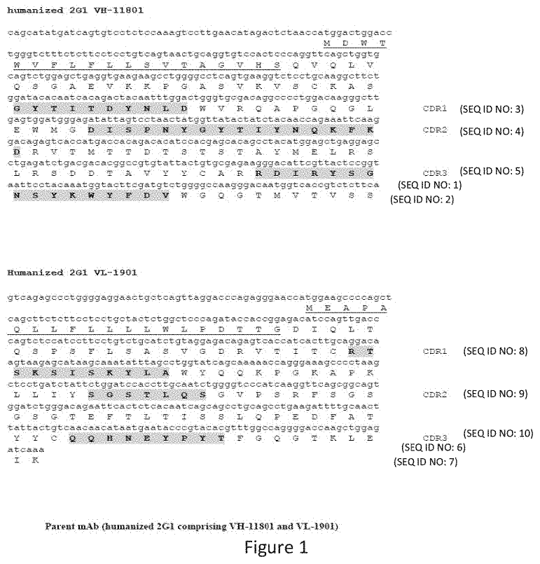

Variants of humanized 2G1 (VH-11801 (SEQ ID NO:2) and VL-1901 (SEQ ID NO:7)) were generated (FIG. 1), with the aim of developing variants with improved binding to C5 at pH 7.4 and reduced binding to C5 at pH 5.8.

The approach described herein is based on the understanding that a mAb's affinity and blocking efficacy measured in vitro does not necessarily correlate with its in vivo half-life, PK or PD. This is, at least in part, because for a soluble antigen that is present in high concentration in the blood, such as C5, forms an immune complex (i.e., mAb bound to antigen) that is targeted for removal from the body. Therefore, generally, high antibody concentrations in the blood are required to block activity of such soluble antigens in vivo.

The approach described herein is based on the understanding that in vivo efficacy of a therapeutic antibody can be enhanced by increasing antibody recycling or half-life (and therefore PK) and by accelerated antigen intracellular degradation by generating mAbs that possess “pH-dependent” binding properties. The desirable property in this regard is that the therapeutic mAb would bind well to the antigen (e.g., C5) at close to neutral pH (˜pH 7.4) which is the pH of the blood. In this way, it effectively blocks the antigen (e.g., C5) activity. The immune complex is then taken up by cells where it moves to the endosome for proteolytic degradation. The pH of early endosome is acidic (˜pH 5.8). So when a therapeutic mAb has poor binding to its antigen at acidic pH, the mAb will dissociate from the immune complex and can then be taken up by the FcRn and returned to the plasma. In this way, only the antigen (e.g., C5) is degraded through the endosome proteolytic pathway, whereas the recycling of the mAb through FcRn contributes to its extended persistence in the plasma.

Due to its propensity of protonation at acidic pH (H+), mAbs containing hisitdine residues in their CDRs may have weakened binding affinity upon protonation at acidic pH. The approach described herein made use of “histidine scanning” of all CDR residues (i.e., substitution of each CDR residue with histidine). Then Octet instrument (Pall ForteBio) was used to measure mAb and C5 dissociation at pH 7.4 and pH 5.8 to identify the histidine-substitution variants that had relatively faster dissociation at pH 5.8 and relatively slower dissociation at pH 7.4.

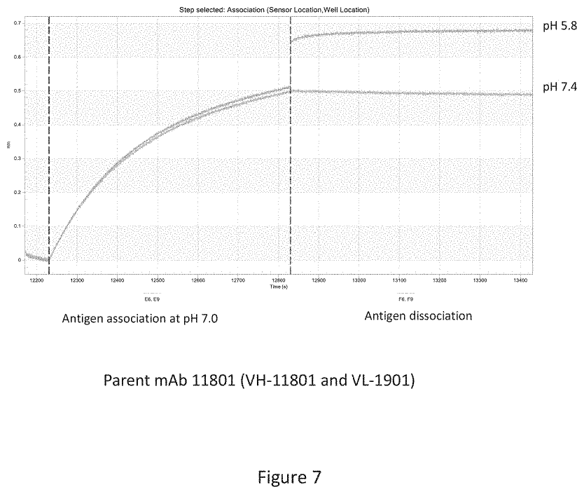

The parental humanized 2G1 mAb (VH-11801 (SEQ ID NO:2) and VL-1901 (SEQ ID NO:7)), appeared to have better affinity for C5 at pH 5.8 (FIG. 7). Single-substitution variants having a histidine substitution at each residue in each of the six CDRs of mAb (VH-11801 (SEQ ID NO:2) and VL-1609 (SEQ ID NO:7)) were generated and evaluated for their pH-dependent binding property. For each variant, a VH and VL plasmid was constructed and then transiently transfected into HEK cells. mAb in the cell culture supernatant was tested for binding at pH 5.8 and pH 7.4.

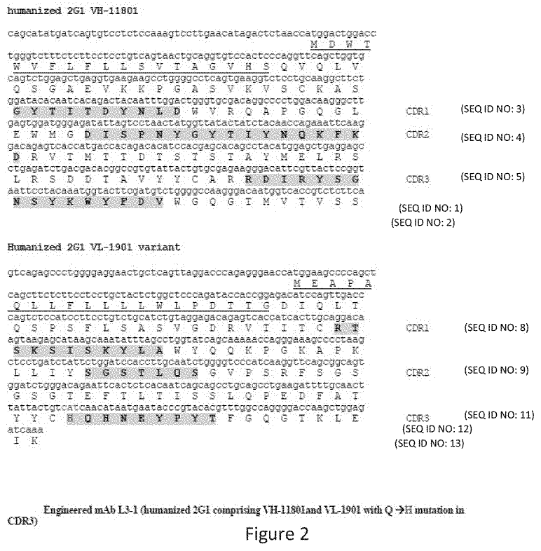

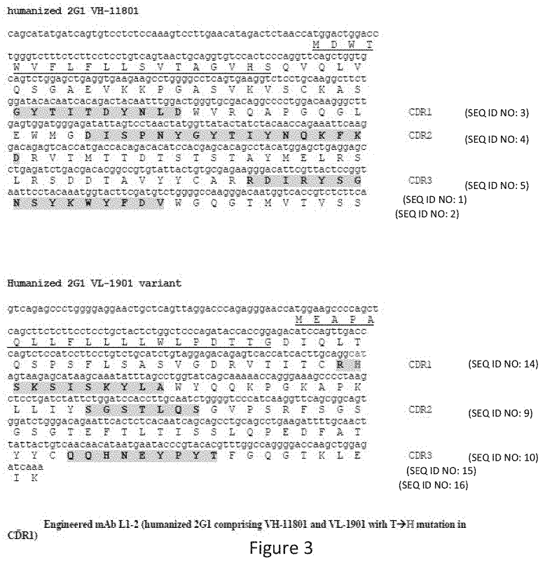

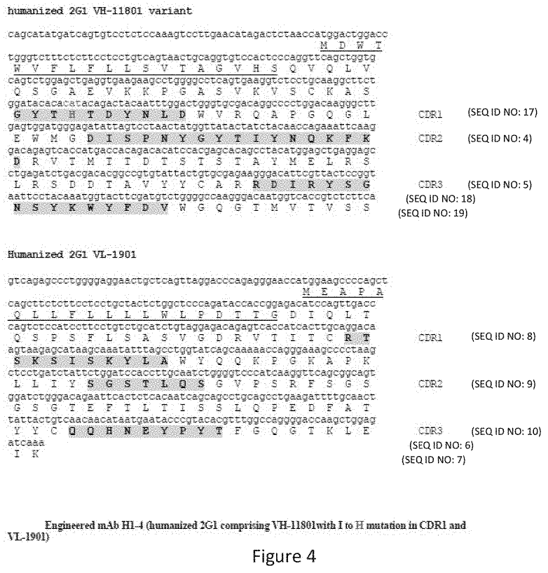

Of these single-substitution variants, three (mAb L3-1, L1-2 and H1-4) exhibited some improvement in pH-dependent binding. The sequences of L3-1 are shown in FIG. 2 (SEQ ID NOs: 1-5, 8, 9, and 11-13). The sequences of L1-2 are shown in FIG. 3 (SEQ ID NOs: 1-5, 9, 10, and 14-16). The sequences of H1-4 are shown in FIG. 4 (SEQ ID NOs: 4, 5, 6-10, and 17-19).

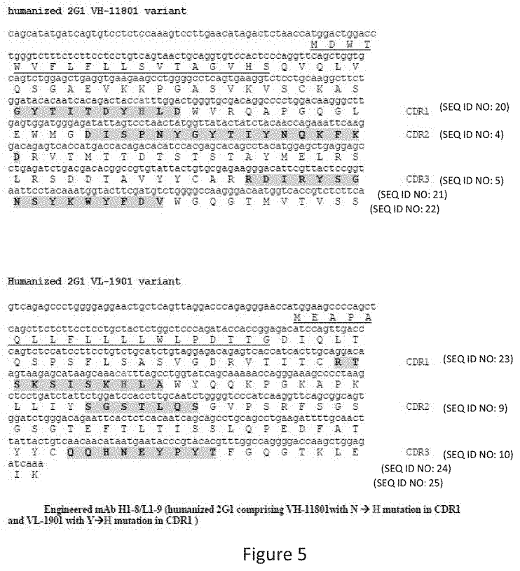

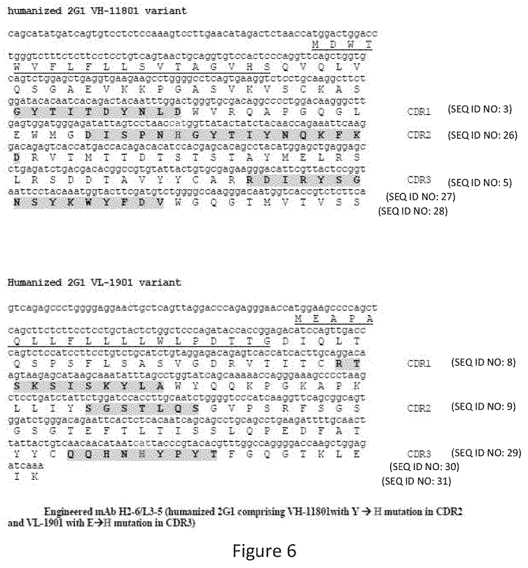

In addition to generating the single-substitution variants, the single-substitution light chains and heavy chains were combined to generate double-substitution variants. Of these double-substitution variants, two (mAb H1-8/L1-9 and H2-6/L3-5) exhibited some improvement in pH-dependent binding. The sequences of H1-8/L1-9 are shown in FIG. 5 (SEQ ID NOs: 4, 5, 9, 10, and 20-25). The sequences of H2-6/L3-5 are shown in FIG. 6 (SEQ ID NOs: 3, 5, 8, 9, and 26-31).

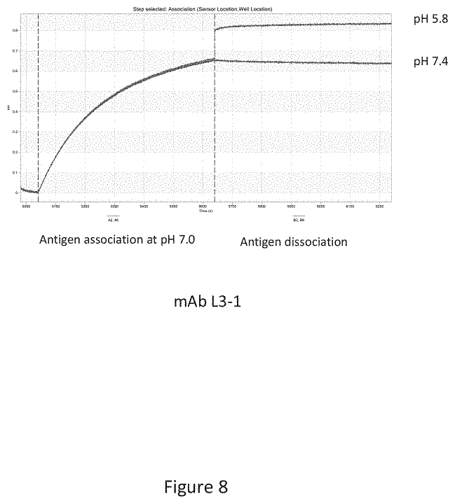

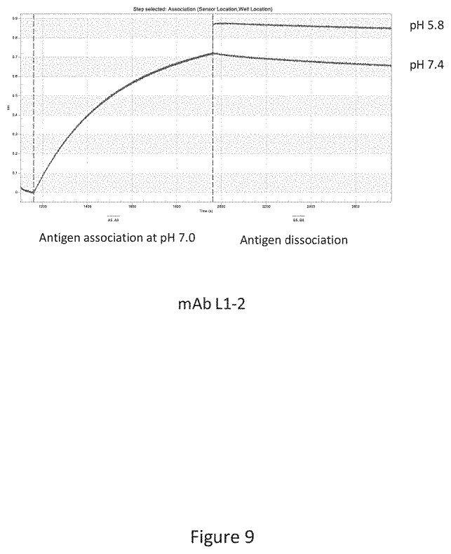

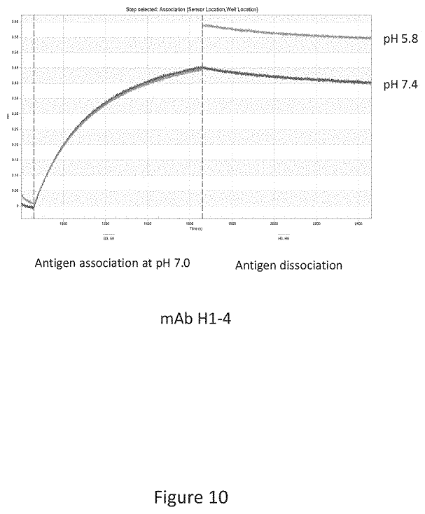

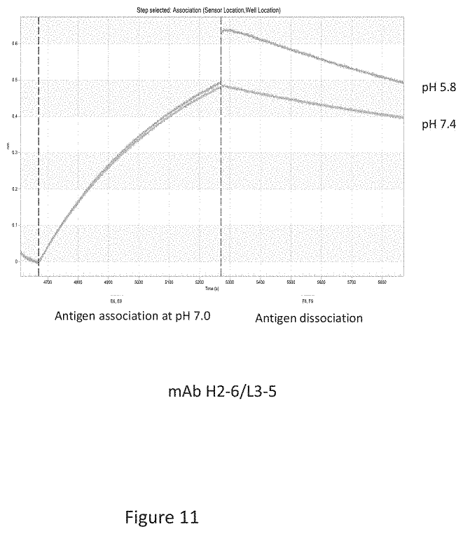

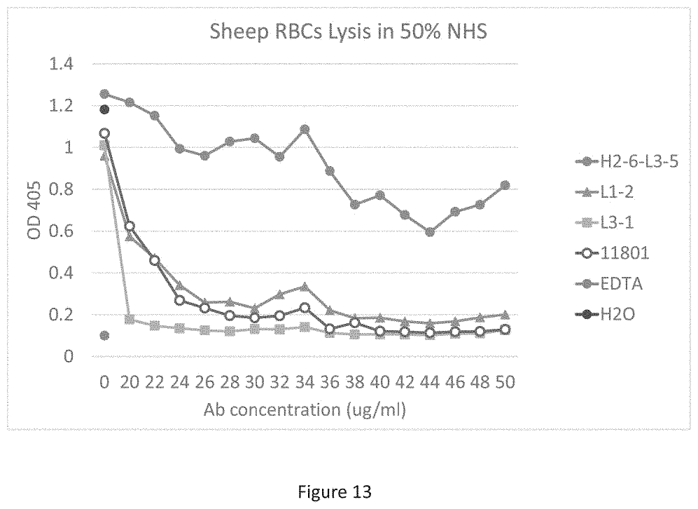

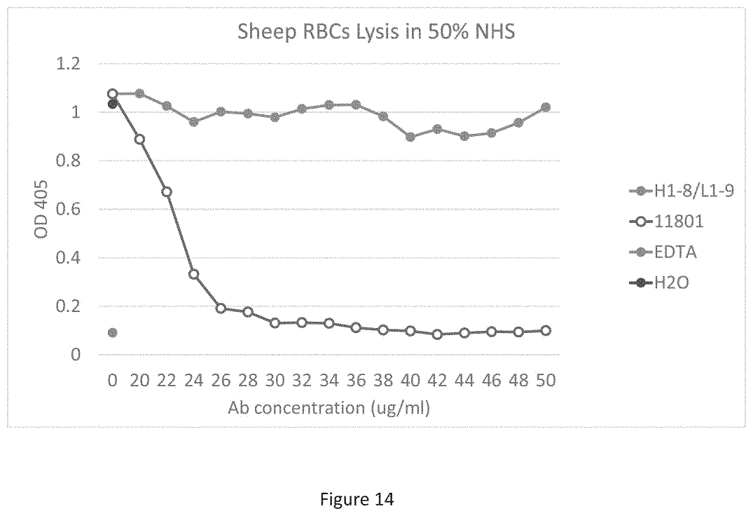

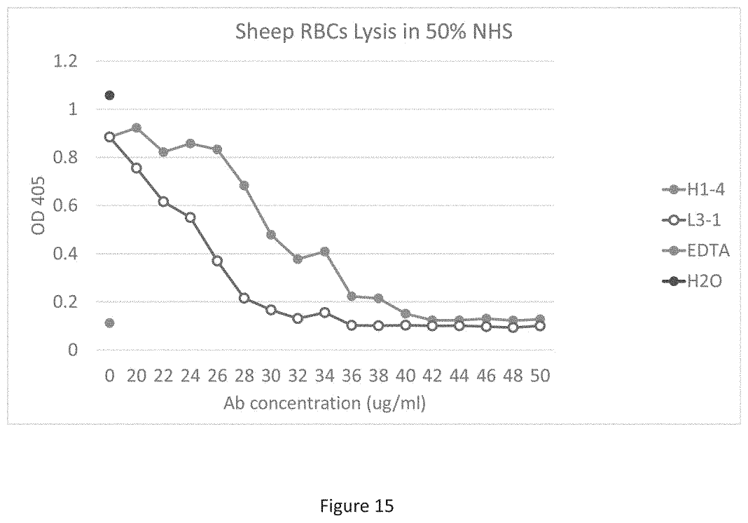

The Octet traces of C5 binding and dissociation of the parental humanized mAb 2G1 with VH-11801 SEQ NO:2 and VL-1901 SEQ NO:7, and the various single and double histidine mutants are shown in FIG. 7 through FIG. 12. Their relative activities in blocking C5 function is compared to the parent humanized 2G1 mAb (VH-11801/VL-1901) and are shown in FIG. 13 and FIG. 14, using a sheep red blood cell lysis assay. Except mAb L3-1 which showed equal or improved activity over the parental humanized 2G1 mAb, all other mutants showed reduced activity. In the case of the two double-substitution variants, their activity is greatly reduced with H1-8/L1-9 essentially lost all C5-blocking activity. This is not surprising because although we observed some desirable faster dissociation at pH 5.8 for mAb H1-8/L1-9 and H2-6/L3-5, their dissociation at pH 7.4 is also dramatically accelerated (which explains the loss of activity).

Even though the above histidine-substituted variants become less active in vitro, their in vivo half-life might be improved and therefore their PK/PD properties could be improved over the parent mAb due to reduced degradation due to pH-dependent binding.

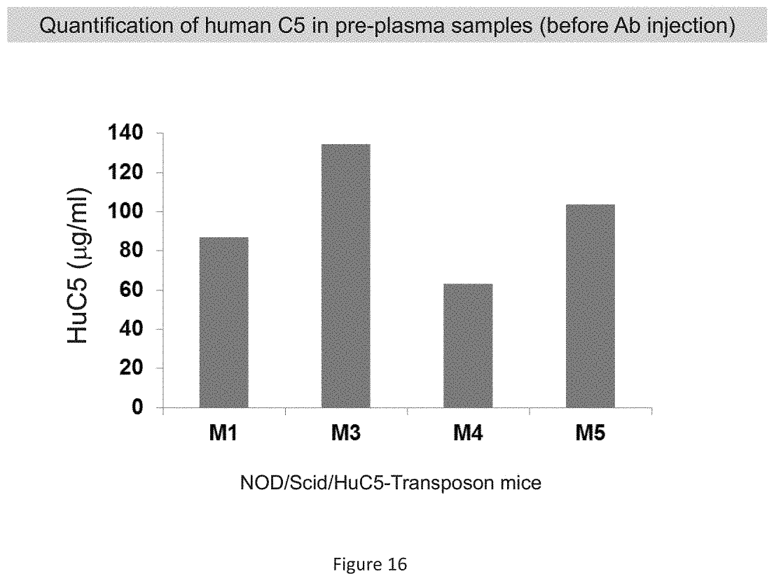

To test in vivo PK/PD, a C5 humanized mouse was developed by making NOD/SCID mice permanently expressing human C5 through hydrodynamic injection via tail vein of a human C5 cDNA containing the Sleeping Beauty transposon elements. NOD mice are naturally deficient in C5, so endogenous mouse C5 will not interfere with pharmacodynamics (PD) assay. The SCID genetic background ensures that the transgenically expressed human C5 will not elicit an immune response against human C5. The C5 humanized mouse was developed by hydrodynamic injection of human C5 plasmid containing the Sleeping Beauty transposon sequence elements for stable genomic integration. Representative data of high level human C5 expression in NOD/SCID mice are shown in FIG. 16. Typically, plasma concentrations of C5 ranging from 50 to 120 μg/mL were observed. This is comparable to C5 concentration in human plasma which is about 80 μg/mL.

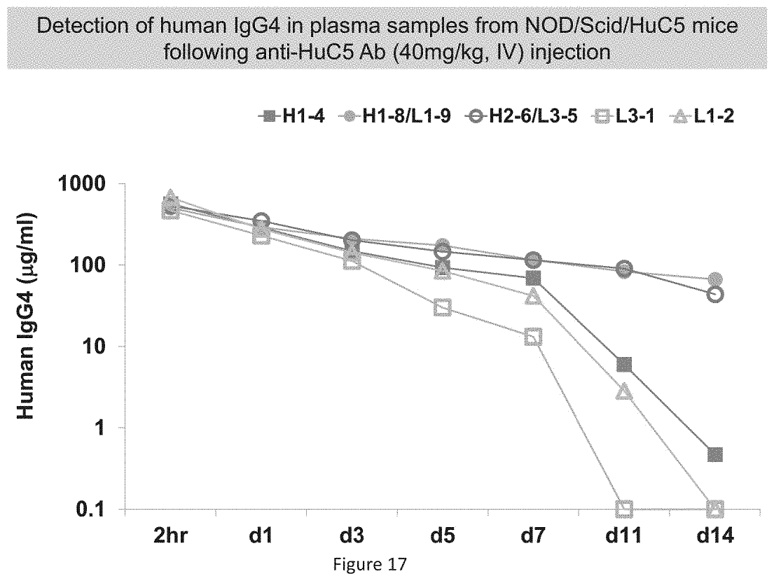

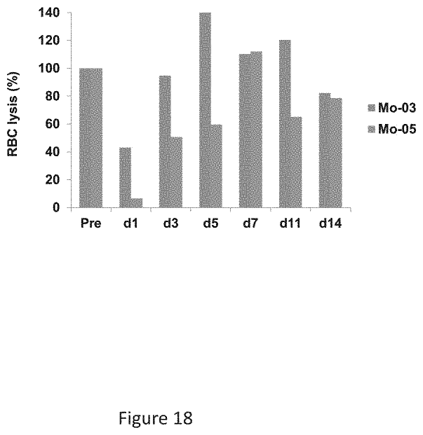

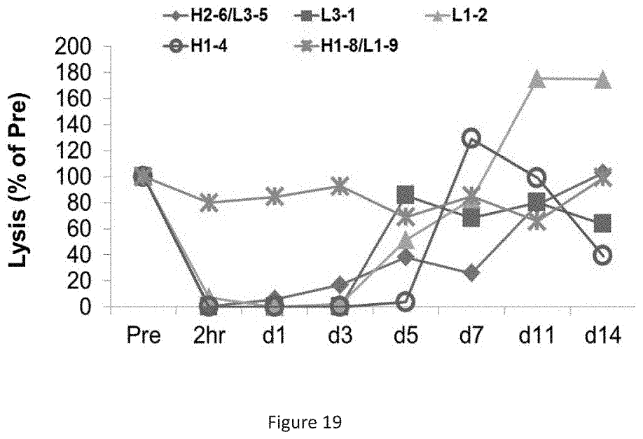

The five histidine-substitution variants (i.e., mAb L3-1, L1-2, H1-4, H1-8/L1-9 and H2-6/L3-5) were assessed for PK/PD. The two double-substitution variants (i.e., mAb H1-8/L1-9 and H2-6/L3-5) exhibited the greatest persistence. Of the three single-substitution variants, mAb H1-4 and L1-2 exhibited better persistence than mAb L3-1 (FIG. 17). Thus, although L3-1 had the best pH 7.4 affinity and in vitro blocking activity for human C5, it has the shortest half-life. Interestingly, except mAb H1-8/L1-9 which showed no blocking activity in vivo, all other variants have improved PD profile over the parent humanized 2G1 mAb (VH-11801, VL-1901) (FIG. 18 and FIG. 19).

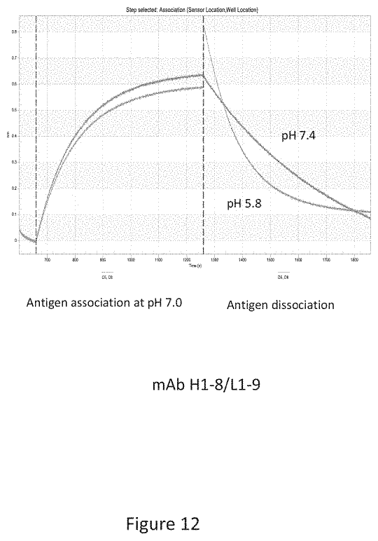

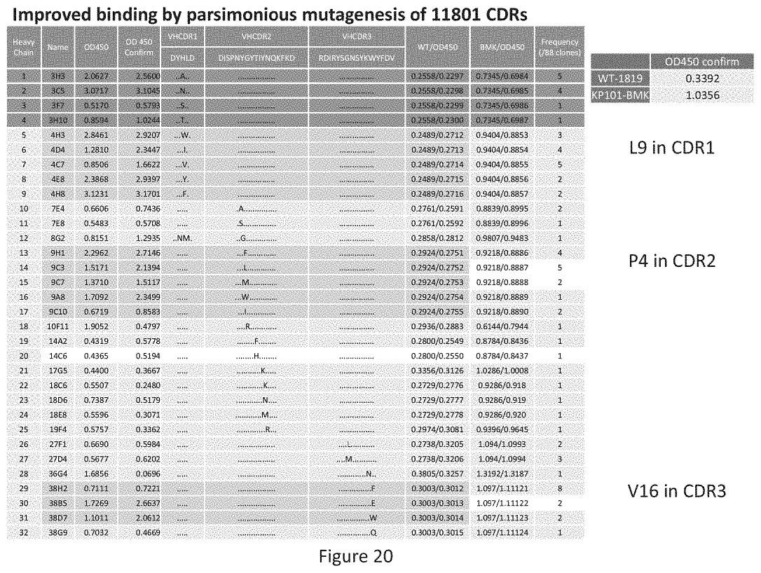

Given that variant mAb H1-8/L1-9 exhibited the best pH-binding differential (i.e., antigen dissociation occurs much faster at pH 5.8 than pH 7.4 (FIG. 12)), but lost much of its C5 blocking activity (because antigen dissociation at pH 7.4 also increased), parsimonious mutagenesis was performed to randomly substitute each residue in the 6 CDRs of H1-8/L1-9 (while conserving the histidine substitutions of H1-8 and L1-9). Three “hot spots” were identified in VH that significantly improved pH 7.4 binding in ELISA plate assays of ScFV constructs. These residues were leucine at position #9 (i.e., L9) in VH CDR1, relative to SEQ ID NO:20, proline at position #4 (i.e., P4) in VH CDR2, relative to SEQ ID NO:4, and valine at position #16 (i.e., V16) in VH CDR3, relative to SEQ ID NO:5. The substitutions exhibiting the greatest improved binding were L9→W9 (i.e., L9W), L9→I9 (i.e., L9I), L9→V9 (i.e., L9V), L9→Y9 (i.e., L9Y), L9→F9 (i.e., L9F), P4→F4 (i.e., P4F), P4→L4 (i.e., P4L), P4→M4 (i.e., P4M), P4→W4 (i.e., P4W), P4→I4 (i.e., P4I), V16→F16 (i.e., V16F), V16→E16 (i.e., V16E) and V16→W16 (i.e., V16W) (FIG. 20).

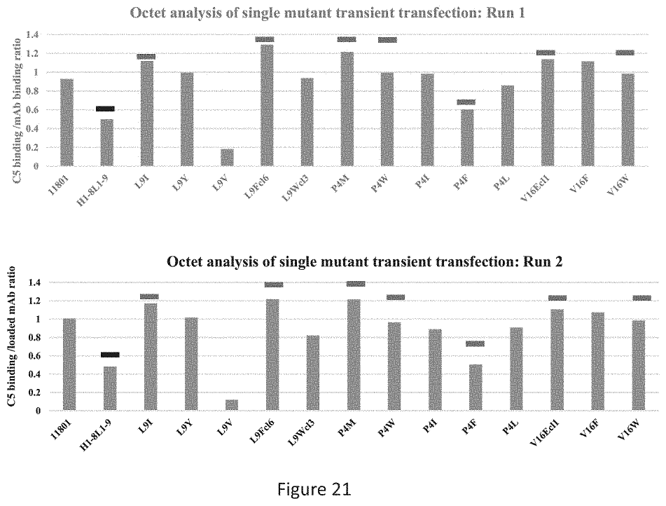

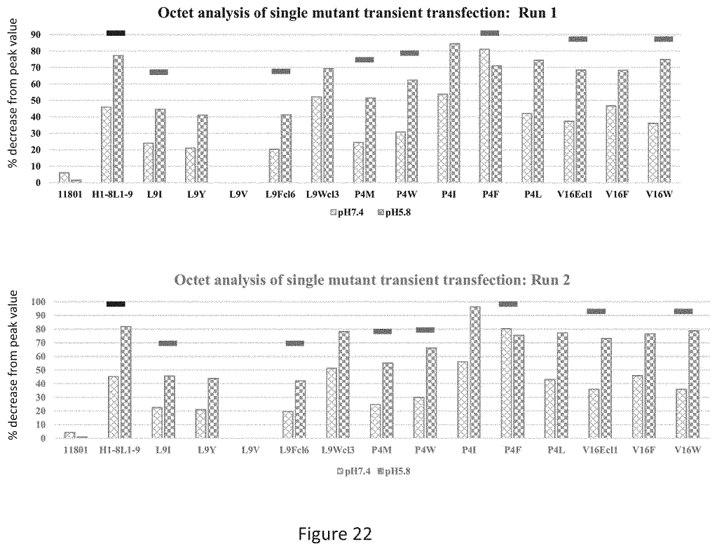

These 13 substitutions (i.e., L9→W9 (i.e., L9W), L9→I9 (i.e., L9I), L9→V9 (i.e., L9V), L9→Y9 (i.e., L9Y), L9→F9 (i.e., L9F), P4→F4 (i.e., P4F), P4→L4 (i.e., P4L), P4→M4 (i.e., P4M), P4→W4 (i.e., P4W), P4→I4 (i.e., P4I), V16→F16 (i.e., V16F), V16→E16 (i.e., V16E) and V16→W16 (i.e., V16W)) were generated and evaluated for their pH-dependent binding property as IgG4. For each variant, a VH and VL plasmid was constructed and then transiently transfected into Expi-CHO cells. mAb in the cell culture supernatant was tested for binding at pH 5.8 and pH 7.4. FIG. 21 and FIG. 22 summarize the C5 binding properties of these variants. FIG. 21 shows the ratio of C5 binding relative to mAb binding signals, and FIG. 22 shows the % decrease of binding signal from peak value in pH 7.4 and pH 5.8 after switching from association phase to dissociation phase. For any given mutant, a high ratio on FIG. 21 is preferred; and a lower % of dissociation at pH 7.4 (crosshatch bars) and higher % of dissociation at pH 5.8 (checkered bars) on FIG. 22 are preferred.

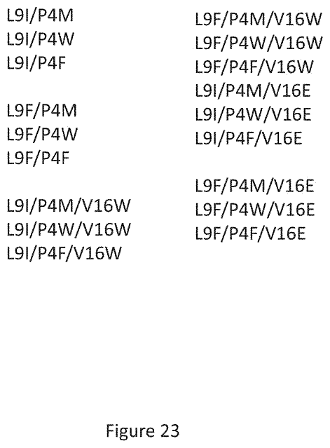

From this screen of the 13 mAb H1-8/L1-9 single histidine substitution variants, seven variants were selected for generating the 18 combination substitution variants listed on FIG. 23 (i.e., L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4M/V16E, L9I/P4W/V16E, L9I/P4F/V16E, L9F/P4M/V16E, L9F/P4W/V16E, and L9F/P4F/V16E). These 18 combination substitution variants were generated and evaluated for their pH-dependent binding property. For each variant, a VH and VL plasmid was constructed and then transiently transfected into Expi-CHO cells. mAb in the cell culture supernatant was tested for binding at pH 5.8 and pH 7.4.

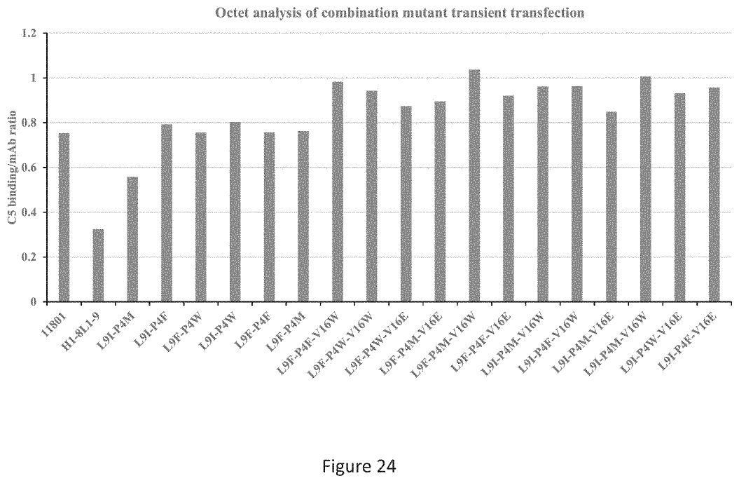

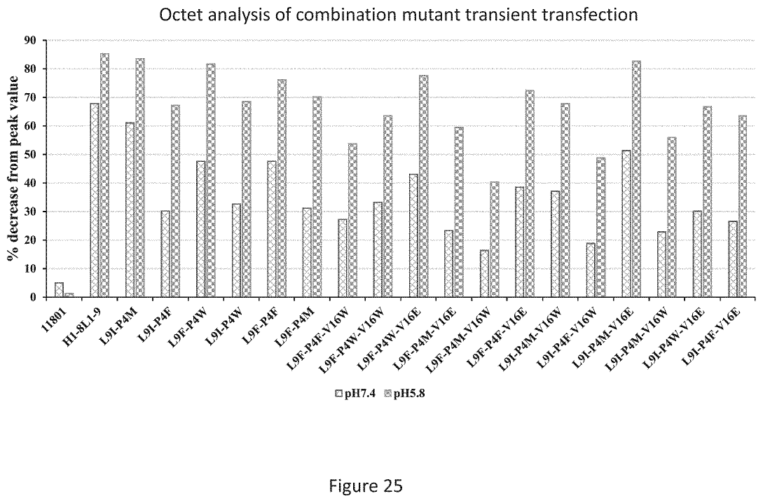

FIG. 24 and FIG. 25 summarize the C5 binding properties of these 18 combination substitution variants. FIG. 24 shows the ratio of C5 binding relative to mAb binding signals, and FIG. 25 shows the % decrease of binding signal from peak value in pH 7.4 and pH 5.8, respectively, after switching from association phase to dissociation phase. For any given mutant, a high ratio on FIG. 24 is preferred; and a lower % of dissociation at pH 7.4 (crosshatch bars) and higher % of dissociation at pH 5.8 (checkered bars) on FIG. 25 are preferred.

### Example 2

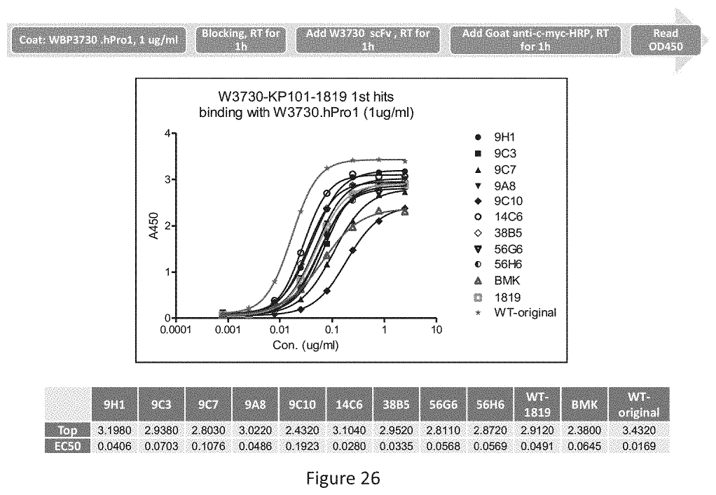

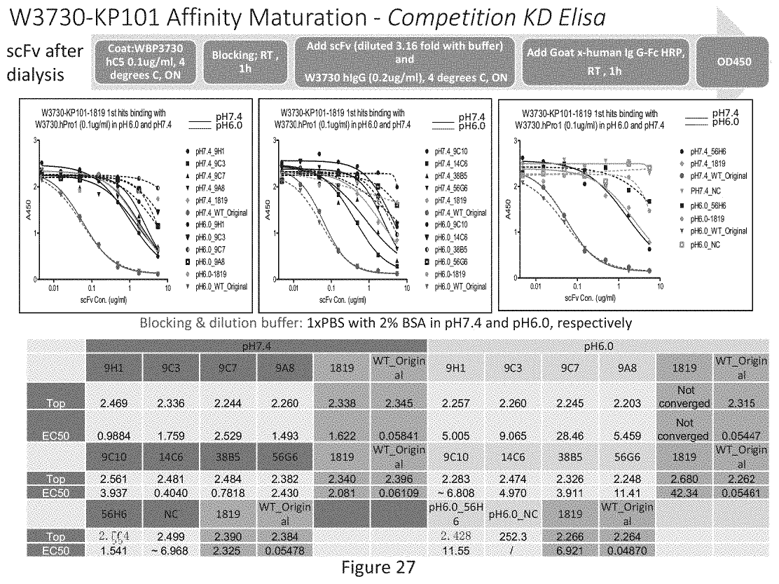

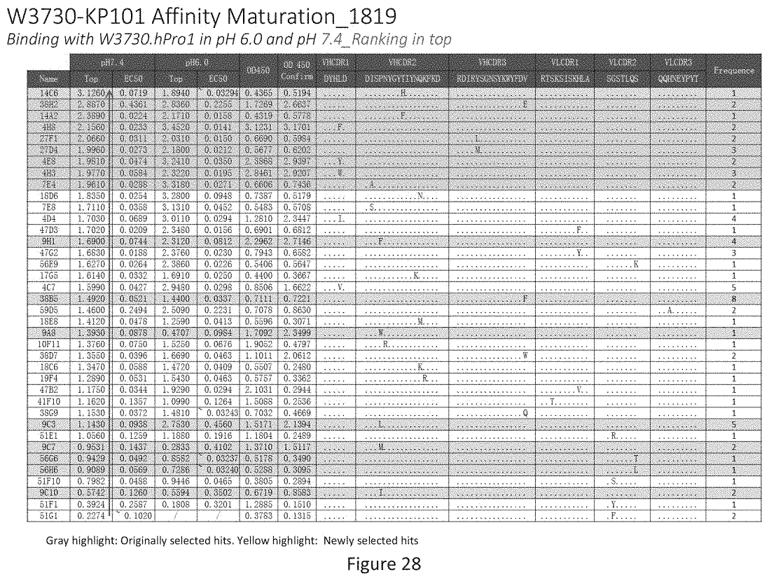

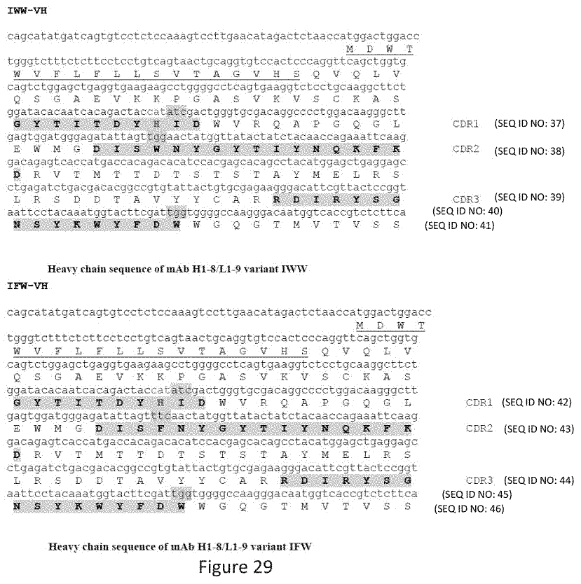

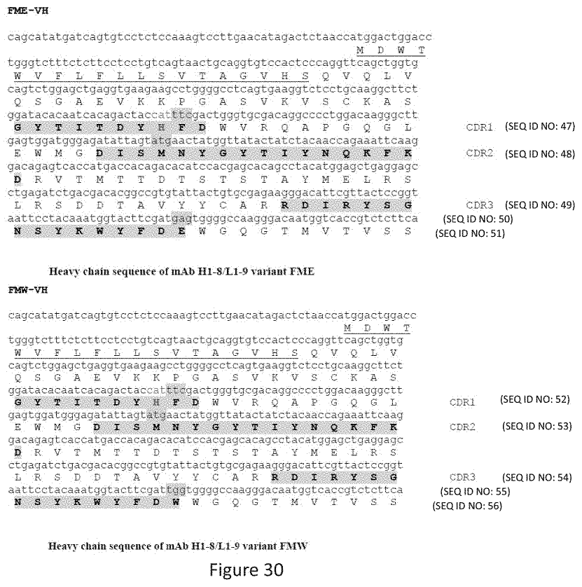

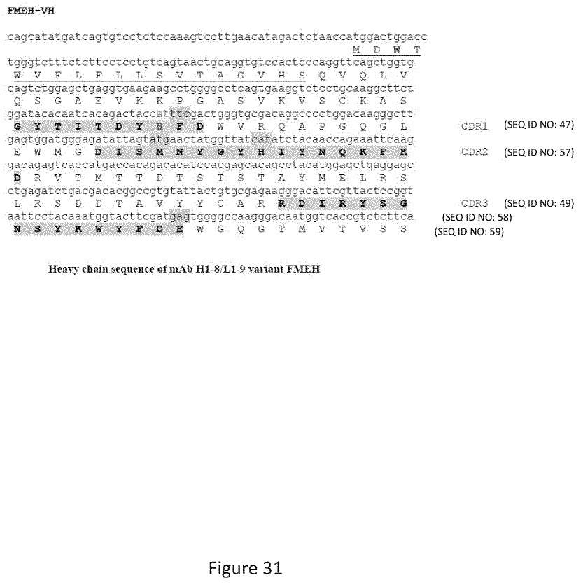

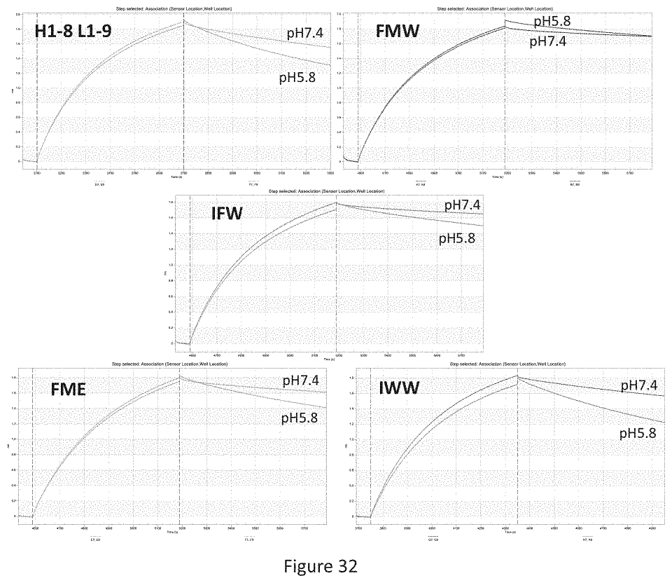

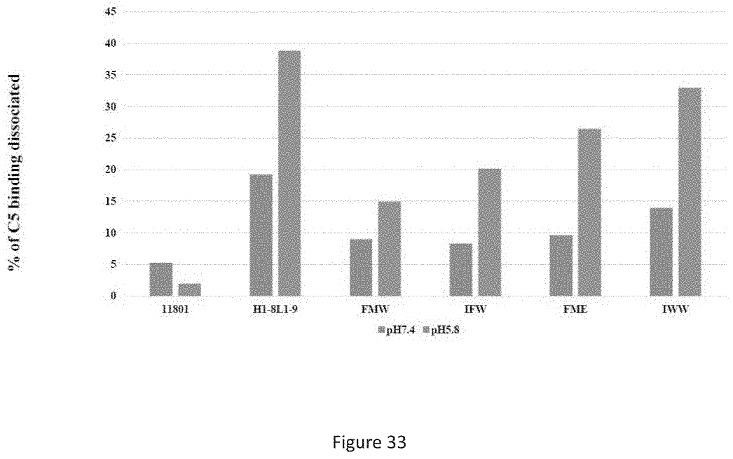

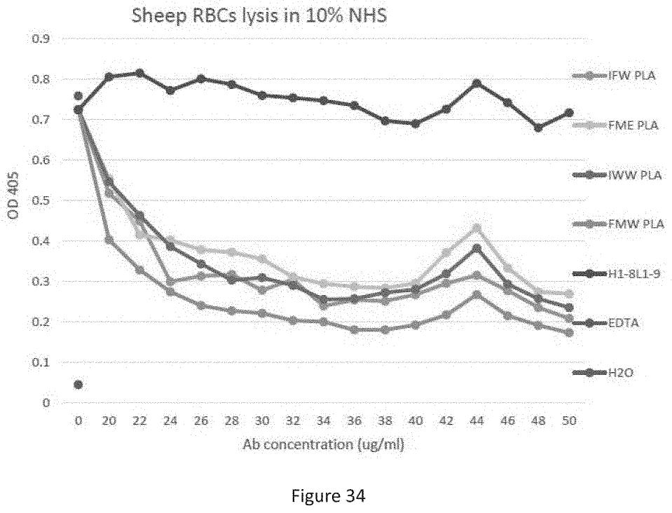

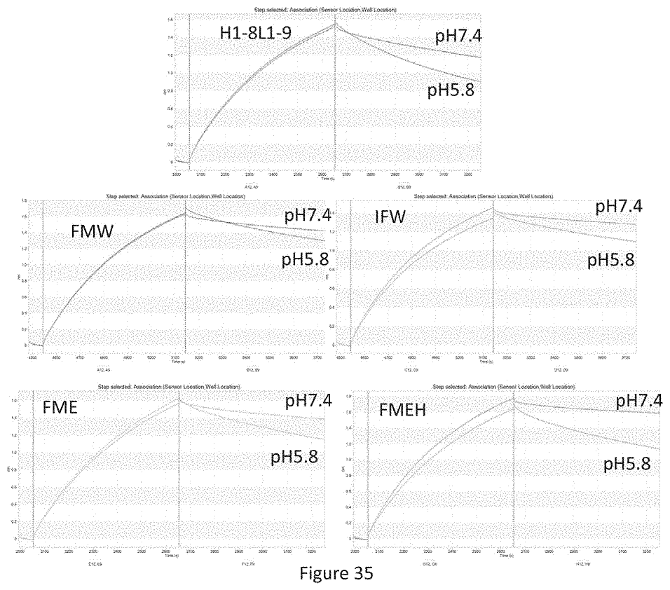

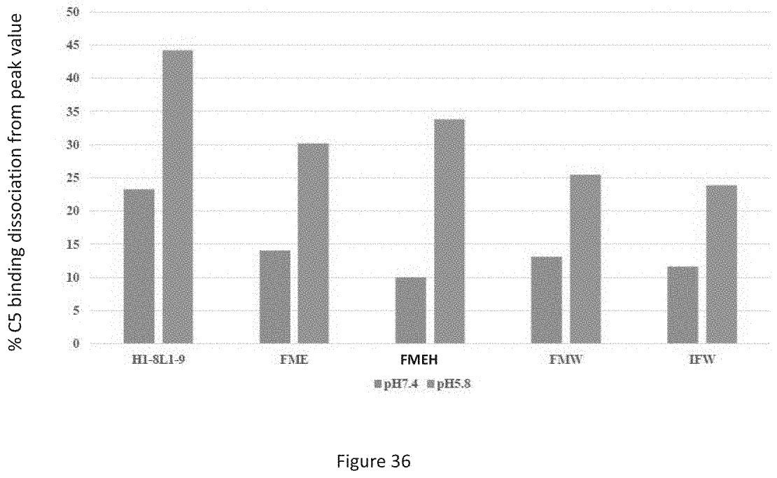

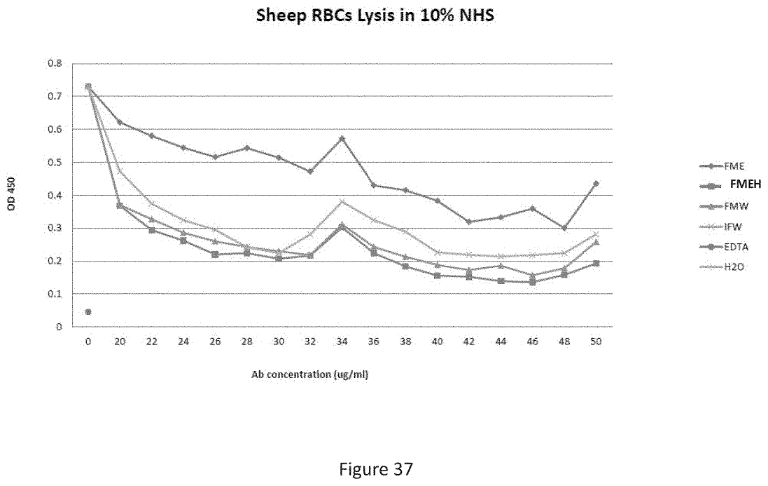

Based on the maximum pH 7.4 vs pH 5.8 binding differential and best pH 7.4 binding, variants IWW, IFW, FME and FMW were selected as the top 4 triple mutant clones of V1-8/L1-9 for further characterization. Additional analysis and data mining of affinity maturation project were performed and identified T to His mutation at CDR2 position 9 as another promising random mutation to repair the pH 7.4 binding of V1-8/L1-9 (FIG. 26 through FIG. 28). IWW, IFW, FME and FMW were selected as the top 4 triple mutant clones of V1-8/L1-9. IWW, IFW, FME and FMW were expressed and the proteins were purified to confirm pH-dependent binding properties. C5 inhibiting activity in hemolytic assay was also tested (FIG. 29, FIG. 30, FIG. 32 through FIG. 34). The above 4 triple mutants were further combined with the new His mutation (CDR2 of VH, position 9, T to H mutation) to produce 4 quadruple mutants (IWWH, IFWH, FMEH and FMWH; e.g., FIG. 31). These new mutants were tested for their pH-dependent binding properties and C5-inhibiting activities. Of them, the quadruple mutant FMEH was identified as an improved mutant over its triple mutant parent molecule FME (FIG. 35 through FIG. 37).

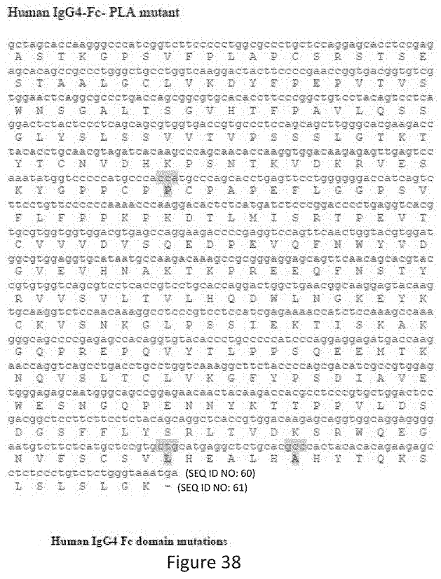

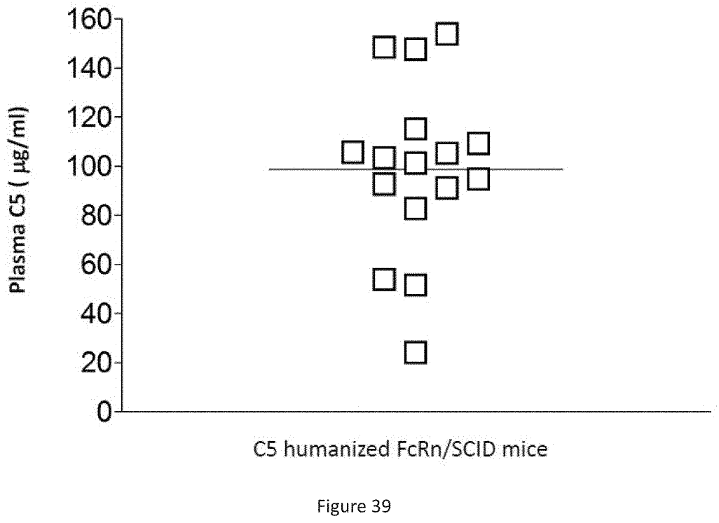

In addition to the C5 humanized mice on NOD/SCID mice, described in Example 1 and generated by hydrodynamic injection to test in vivo PK/PD of some of the engineered mutants of anti-C5 mAbs, C5 humanized mice on SCID/FcRn transgenic mice (human FcRn transgenic and mouse FcRn knockout) background was also generated. The latter strain enabled the testing of the combined effect of pH-dependent anti-C5 mAb mutants with further engineered Fc domain of IgG4 antibody construct (three mutations in Fc domain) (FIG. 38 and FIG. 39).

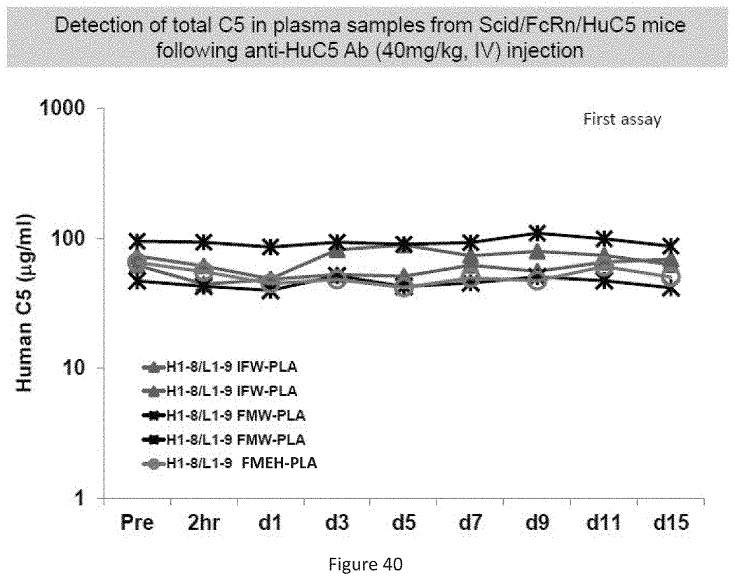

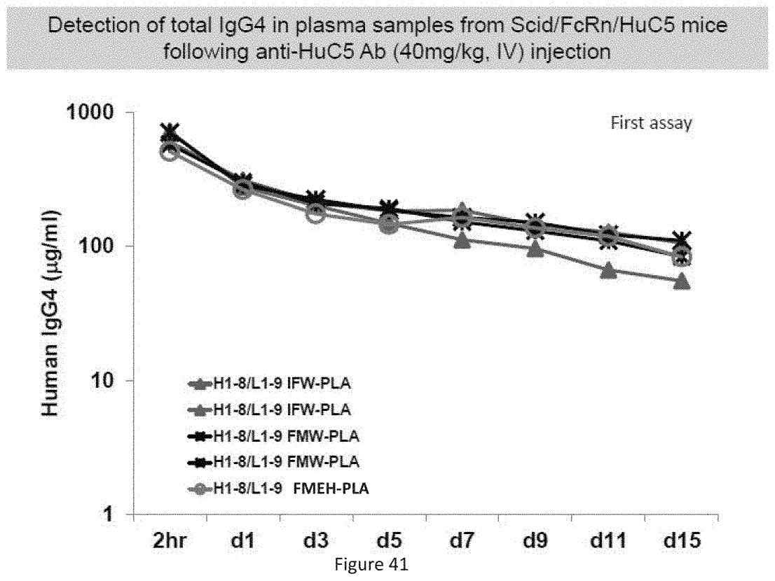

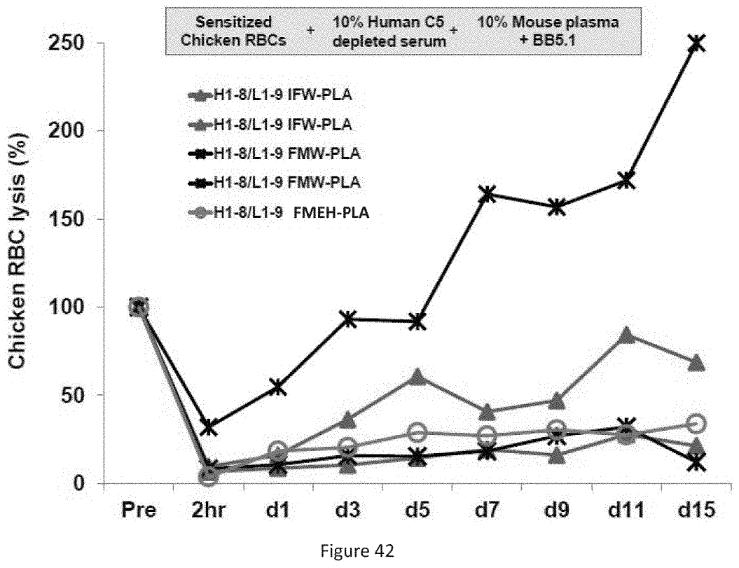

Using C5 humanized mice on SCID/FcRn transgenic background, PK/PD of the H1-8/L1-9 triple mutants IFW, FMW, and FMEH was also tested in IgG4 format with three residue mutations in Fc domain (PLA). The data showed significantly improved PK/PD activity over the original humanized anti-C5 mAb 11801 (from which H1-8/L1-9 was derived) (FIG. 40 through FIG. 42).

### Example 3

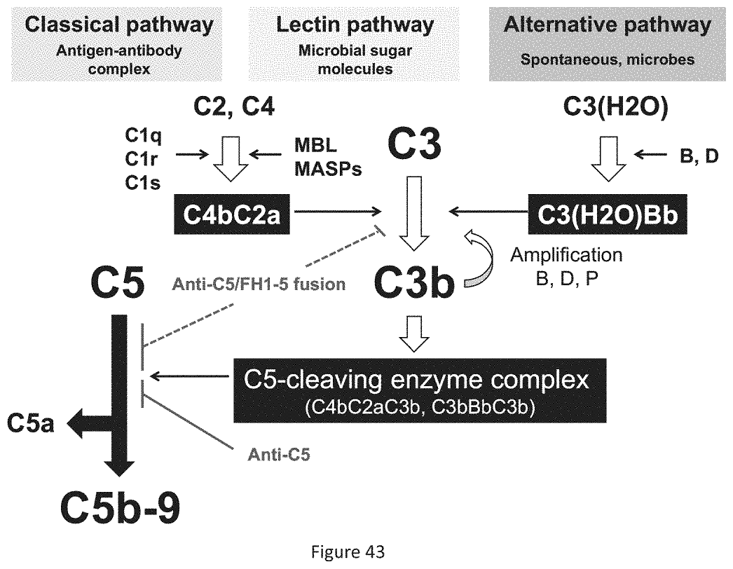

Complement activation involves a cascade of target recognition and proteolytic cleavage. It can be activated via three different pathways, all of them converge at the C3 activation step (FIG. 43). These pathways are the classical, alternative and lectin pathways (FIG. 43). The CP is activated by antigen-antibody complex and involves the sequential activation of C1 and C4/C2 before merging with other pathways at the C3 activation step. The LP is triggered by certain pattern recognition molecules, such as MBL, collectins and ficolins upon their binding to microbial surface sugar molecules. It involves the activation of MASPs which then cleaves C4/C2 and joins the other pathways at the C3 activation step (FIG. 43). The AP is constitutively active at a low level due to spontaneous hydrolysis and activation of C3 to produce C3(H2O). The latter can associate with factor B, and upon proteolytic activation by factor D, produces the initial C3 cleaving enzyme complex C3(H2O)Bb (FIG. 43). In the absence of regulatory proteins, the product of C3(H2O)Bb complex, C3b, can associate with factor B in the same way as C3(H2O) does, and thus starts another cycle of self-amplifying C3 activation.

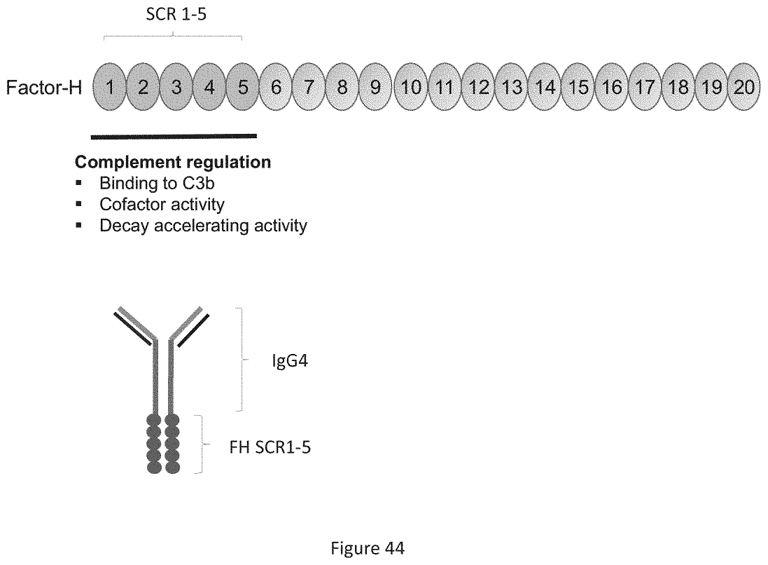

Activation of C3, by any of the three pathways, will invariably trigger the alternative pathway. Thus, in many complement-mediated disease, inhibiting the alternative pathway activation is the key. C3 activation also leads to the generation of C5-cleaving enzyme complexes and initiates the terminal complement activation pathway, culminating in the production of the potent pro-inflammatory mediator C5a and the membrane attack complex C5b-9 which can cause cell lysis and death. To prevent complement from causing indiscriminate injury, host cells express a number of membrane-anchored regulators that function to block complement activation and amplification. Some of these regulators, including decay-accelerating factor (DAF, CD55) and MCP, work to inhibit C3 activation, while others such as CD59 work at other steps of the complement activation cascade. In addition to membrane-anchored complement regulators, there are also fluid phase regulators in the blood which act to preferentially protect the host tissues. The fluid phase inhibitors include FH and factor I (FI), which are critical inhibitors of the alternative pathway of complement activation, and C4BP and C1 inhibitor (C1INH) which inhibit the classical pathway complement activation. Both fluid phase and membrane-anchored complement regulatory proteins are often composed of multiple conserved SCR domains. For example, FH is composed of 20 SCRs (FIG. 44) and the extracellular segment of DAF (CD55) contains 5 SCR domains.

FH inhibits alternative pathway complement activation by serving as a cofactor for FI-mediated cleavage of C3b and by accelerating the decay of C3bBb complex, both mechanisms prevent the amplification loop of the AP. Structure-function studies have revealed that within FH, the complement regulatory activity is located to SCR1-5, whereas other SCR domains, particularly SCR19-20 play a key role in recognition of host cells and surface-deposited C3b. Likewise, the extracellular SCR domains of DAF (CD55) and MCP (CD46) have been shown to be responsible for their complement inhibiting activity and soluble forms of DAF and MCP or their murine homologs have been demonstrated to have complement regulating activities. Mutations leading to absent expression or defective function of complement regulatory proteins are associated with several complement-dependent diseases. For example, gene mutations in enzymes involved in GPI anchor biosynthesis result in the lack of expression of DAF and CD59 on affected hematopoietic stem cells, and corresponding red blood cells (RBC), platelets and leukocytes, of PNH patients. This leads to alternative pathway-mediated complement attack and lysis of affected PNH RBCs and platelet activation, leading to devastating disease pathologies such as anemia and thrombosis in PNH patients. Likewise, mutations in the genes encoding FH and MCP are associated with aHUS. Both PNH and aHUS are now treated in the clinic with a humanized anti-C5 mAb drug Eculizumab as standard therapy. However, there are still significant unmet medical needs and it remains desirable to develop more effective and more convenient anti-complement drugs, both in the treatment of PNH and aHUS and in other complement-mediated diseases.

One of the challenges in developing drugs targeting complement proteins are their high plasma concentration and/or fast turnover. For example, the plasma concentrations of human C3 and C5 are approximately 1 mg/mL and 80 ug/mL, respectively. This means that inhibitors for such proteins need to be administered at a high dose and/or frequently. Indeed, the anti-C5 mAb drug Eculizumab is required to be given every two weeks through intravenous injection in PNH and aHUS patients at a maintenance dose of 900 mg and 1200 mg, respectively. Although a longer lasting second generation anti-C5 mAb Ravulizumab has been developed to lessen the injection frequency to every 8 weeks, the maintenance dose of Ravulizumab has increased to 3600 mg per injection. Furthermore, neither Eculizumab nor Ravulizumab was able to normalize LDH and hemoglobin levels in PNH patients. In PNH patients on standard Eculizumab therapy, breakthrough lysis are frequently observed and 20-30% patients are still transfusion-dependent. These unmet medical needs in PNH patients are related to the fact that deficiency of DAF and CD59 on affected blood cells makes them susceptible to C3 activation as well as MAC-mediated injury. Although anti-C5 mAbs, such as Eculizumab and Ravulizumab, can inhibit C5-mediated hemolysis, they do not prevent C3 activation on affected RBCs and as a result, C3b opsonization of RBCs still happens and this leads to the well-recognized phenomenon of EVH, a process caused by phagocytosis of C3b-opsonized RBCs in the reticuloendothelial system. In addition, studies have shown that blocking complement activation on RBCs at the C5 step has its limitation in efficacy because if too many C5 convertases are already assembled on the cell surface, it becomes impossible to block C5 cleavage completely with mAbs of a finite avidity. This may explain the breakthrough lysis phenomenon in PNH patients treated with Eculizumab and why anti-C5 mAbs are not capable of preventing complete hemolysis of rabbit and PNH RBC cells in ex vivo assays, as both PNH and rabbit RBCs are exceedingly sensitive to C3 complement activation via the AP and can easily assemble abundant C5 convertases on the surface. Although there are ongoing efforts to target C3 activation in complement-dependent diseases, e.g., through the use of C3-inhibitory cyclic peptides or recombinant short variants of FH, such molecules have very poor pharmacokinetics and require large and frequent (e.g., daily) dosing.

This invention, in part, describes improvement in the humanized anti-C5 mAb drug by introducing mutations to VH and VL in order to gain pH-dependent binding to human C5 to improve its pharmacokinetics, but without compromising the C5 blocking activity of the mAb. These mutations in the VH and VL, when combined with IgG4 Fc domain mutations, produced significantly improved pharmacokinetics and pharmacodynamics as assessed in C5 humanized mice on SCID/human FcRn transgenic mice.

The strategy of this invention is based, in part, on the concept of an anti-C5 mAb-FH fusion protein. SCR domain 1-5 was used in FH which, as introduced above, are the domains involved in regulating C3 activation. The goal was not to make a fusion protein with the full-length FH because it is a very large and complex glycoprotein and a full fusion protein with anti-C5 mAb would have manufacture challenges. The N-terminal SCR 1-5 of FH was attached to the C-terminal of the Fc domain of human IgG4 (FIG. 44). There is no linker added between Fc and FH fragment, after the last amino acid of IgG4 Fc, it is linked directly to the N-terminal sequence of mature FH. This anti-C5 mAb fusion protein is bi-functional, it functions as a regular anti-C5 mAb to block C5 activity and the FH domain acts as a C3 complement activation inhibitor.

The other important advantage of this construct is that it used long-lasting (re-cycling) anti-C5 mAbs to construct the FH fusion proteins. The long lasting anti-C5 mAbs are engineered to have pH-dependent binding to C5 (faster dissociation from C5 at pH 5.8 than at pH 7.4), coupled with IgG4 Fc domain mutations (S228P, M428L and N434A, referred to as PLA).

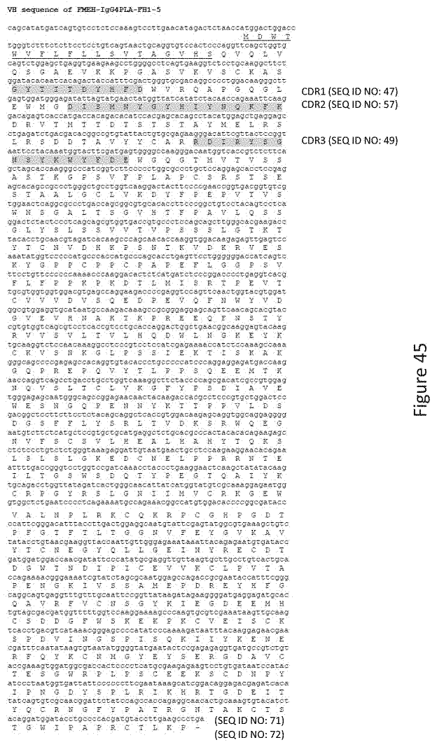

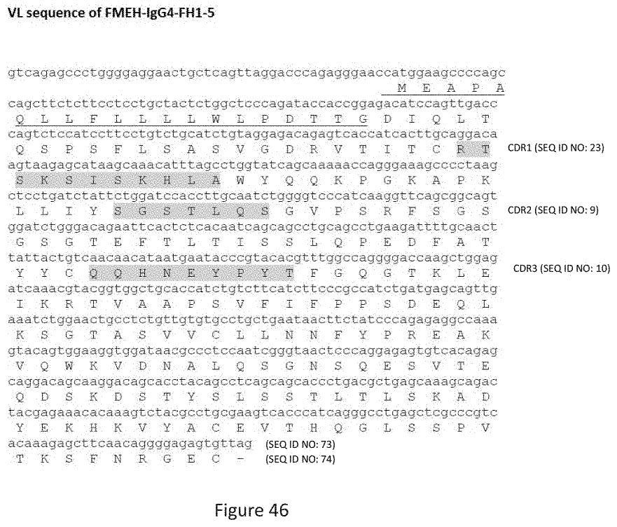

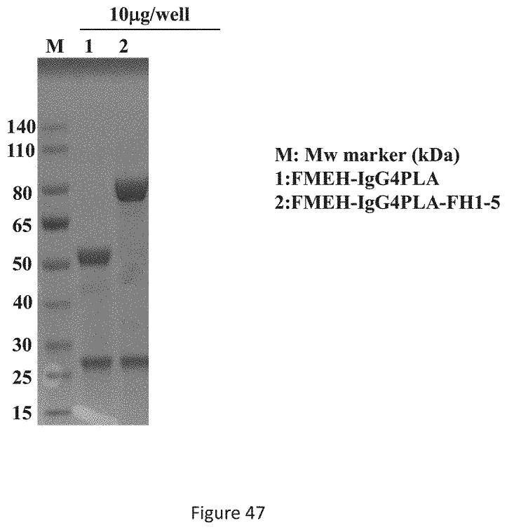

The data presented herein includes the VH and VL sequences of an anti-C5 mAb-FH fusion protein based on the anti-C5 mAb FMEH-IgG4PLA (FIG. 45 and FIG. 46). SDS gel showed the expression VH chain of fusion proteins displaying the expected larger size and had good integrity (FIG. 47).

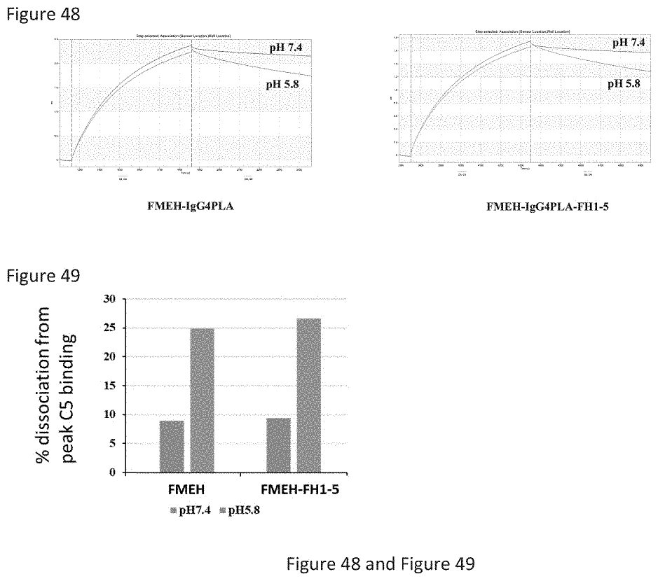

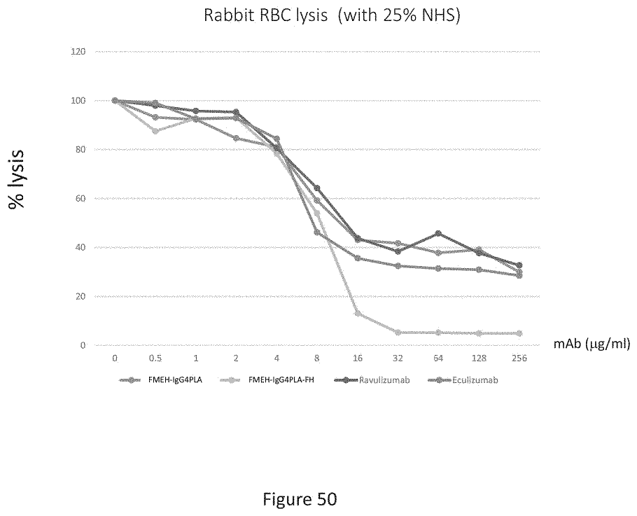

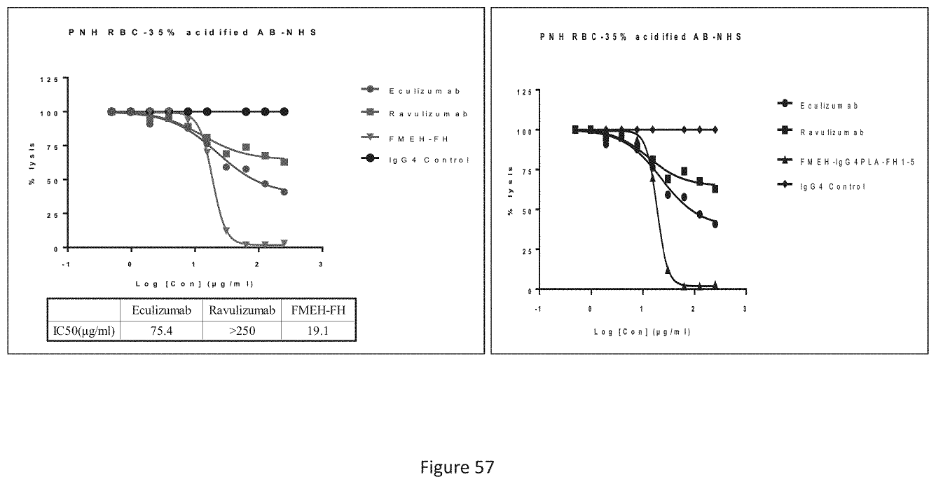

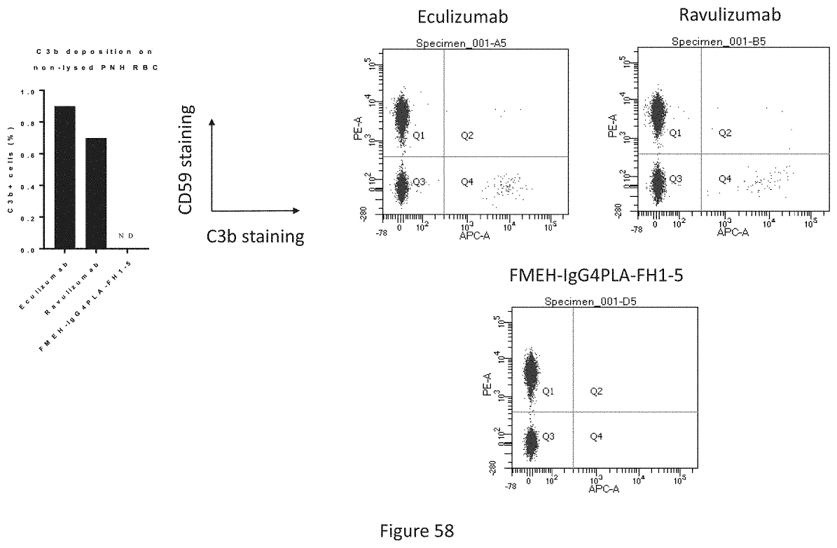

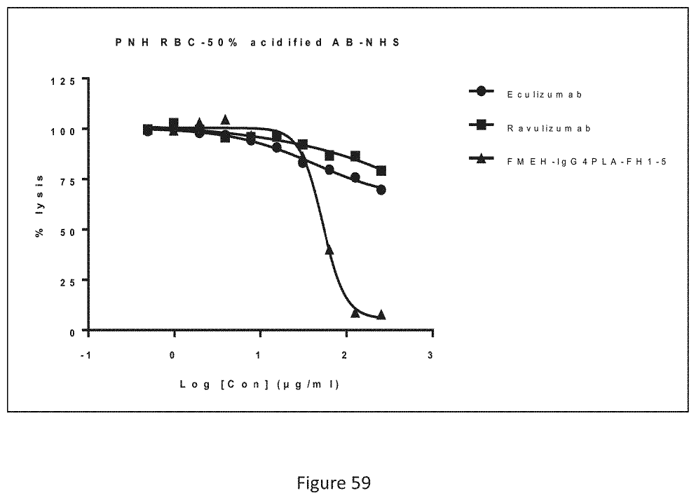

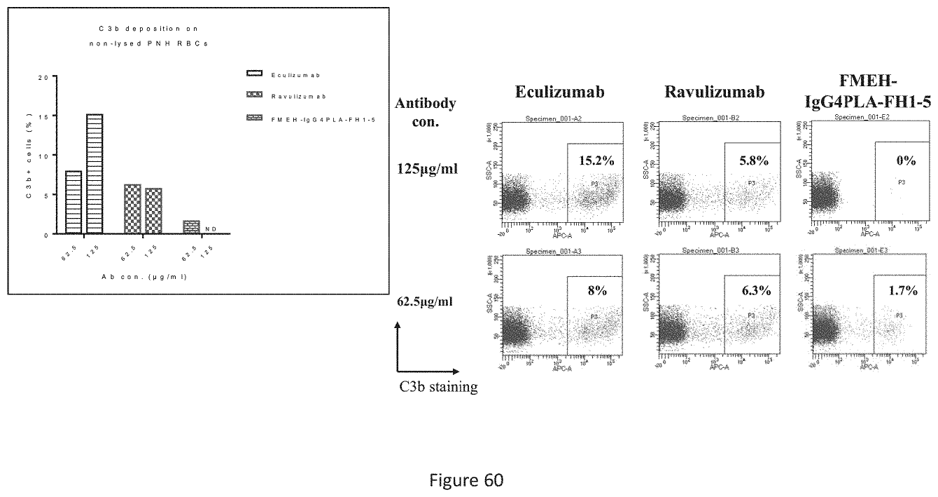

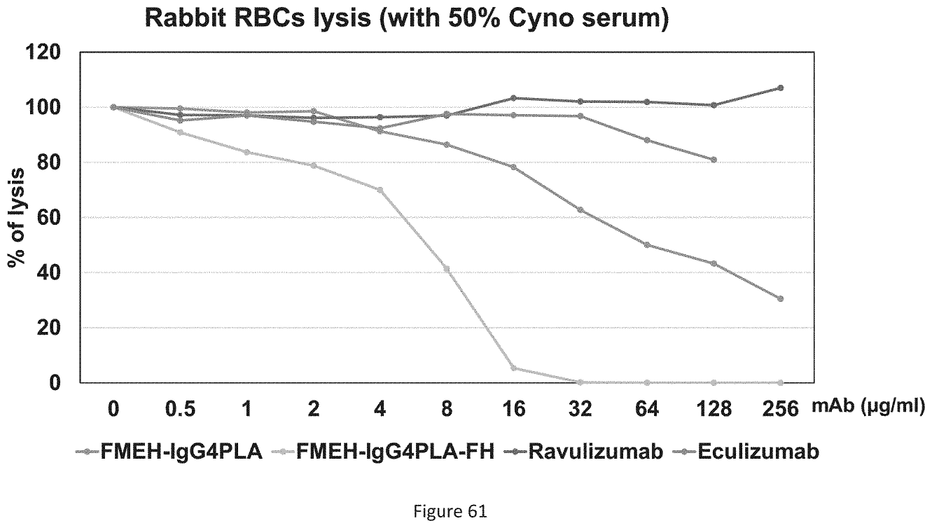

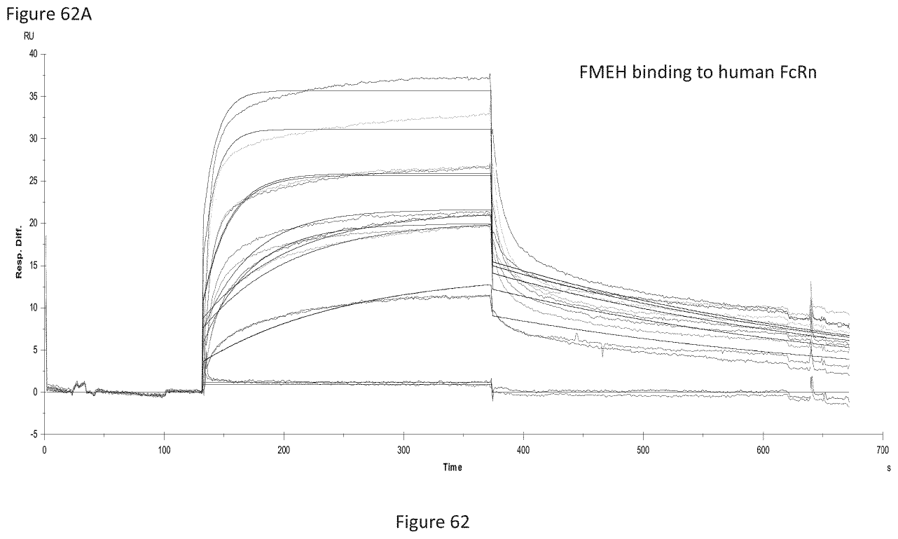

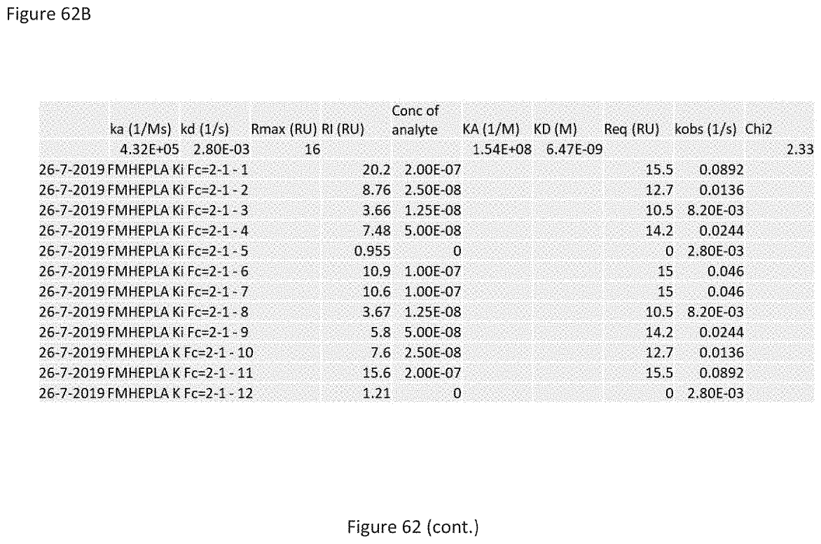

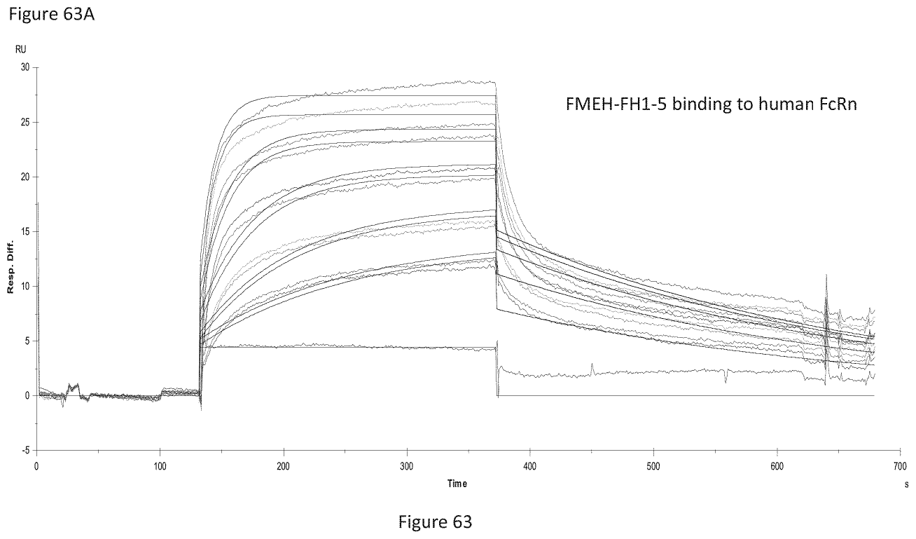

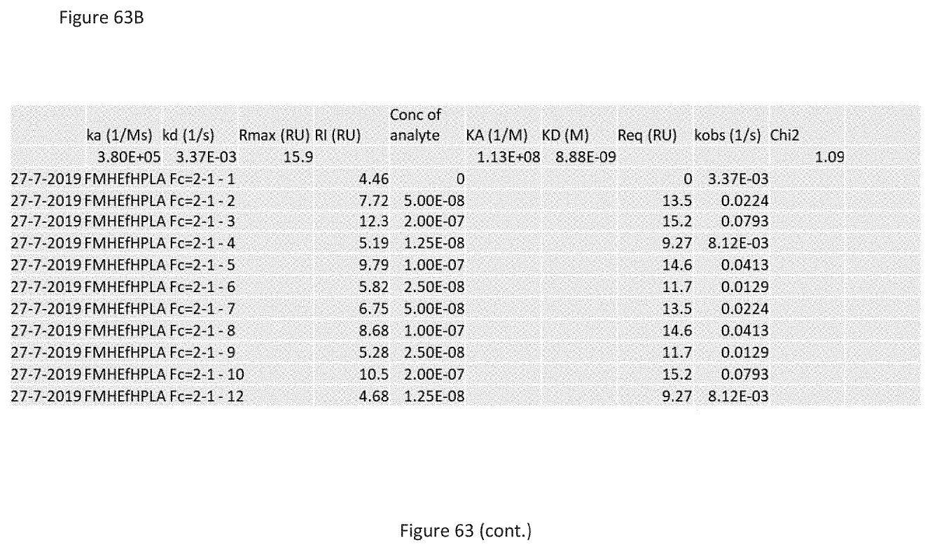

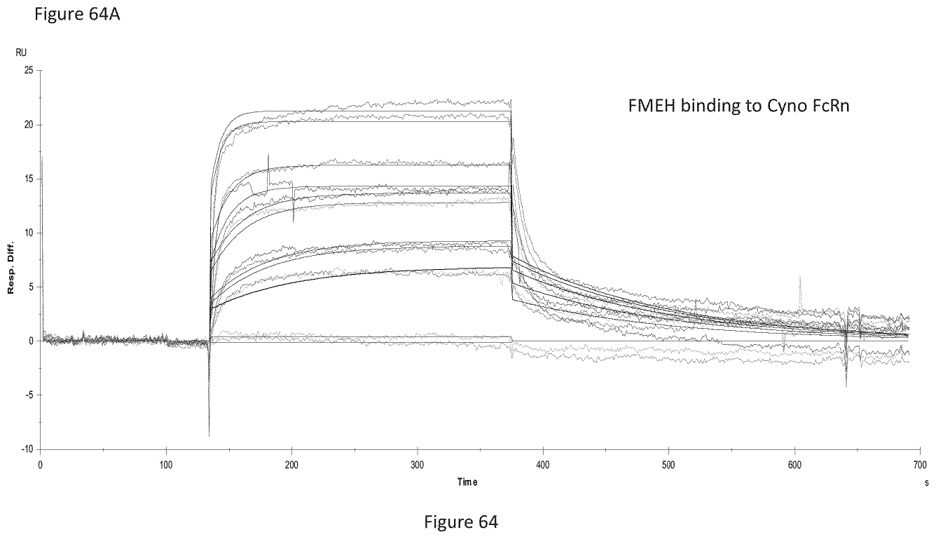

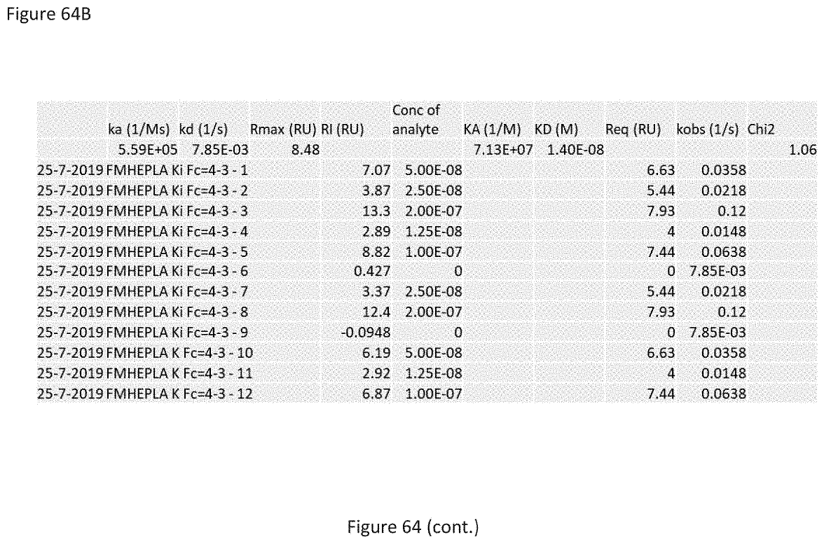

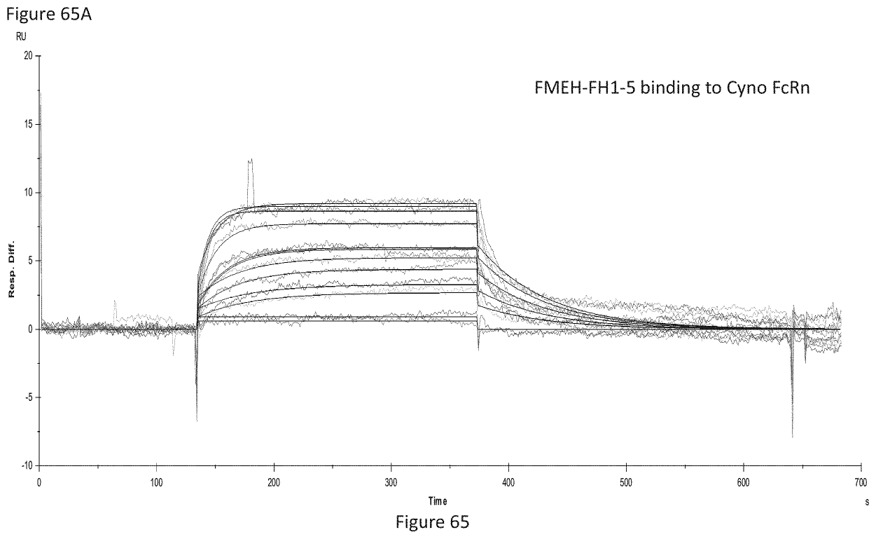

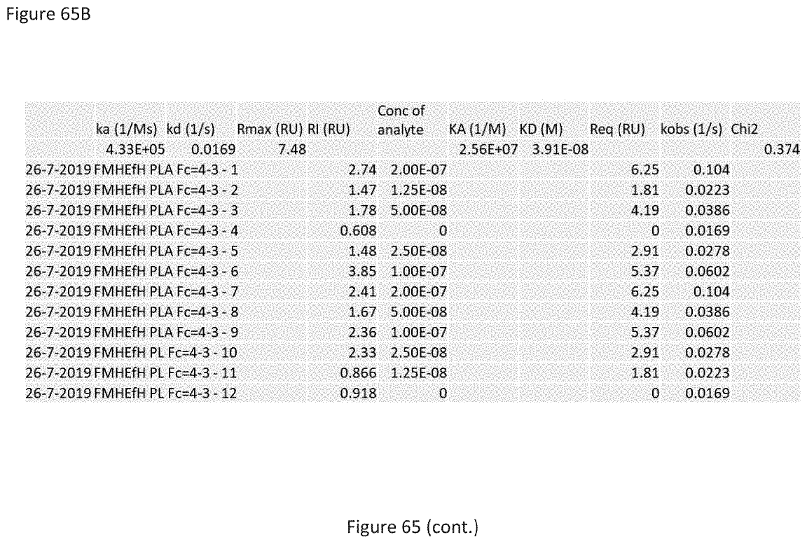

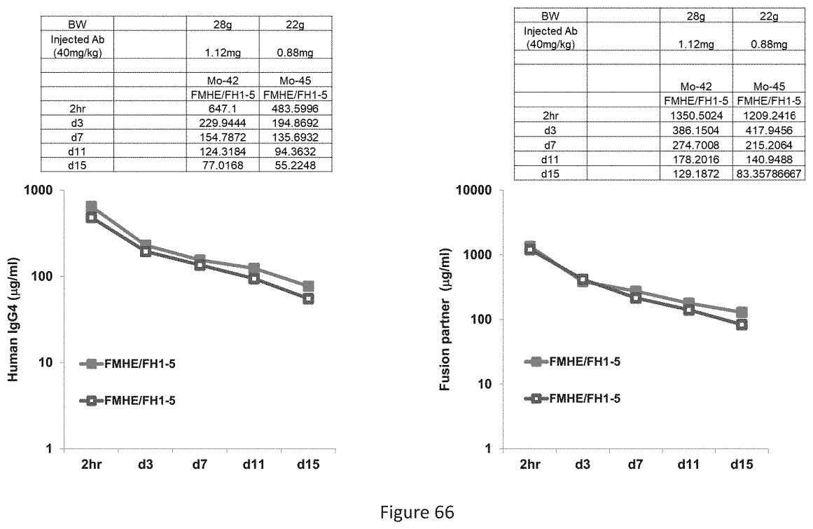

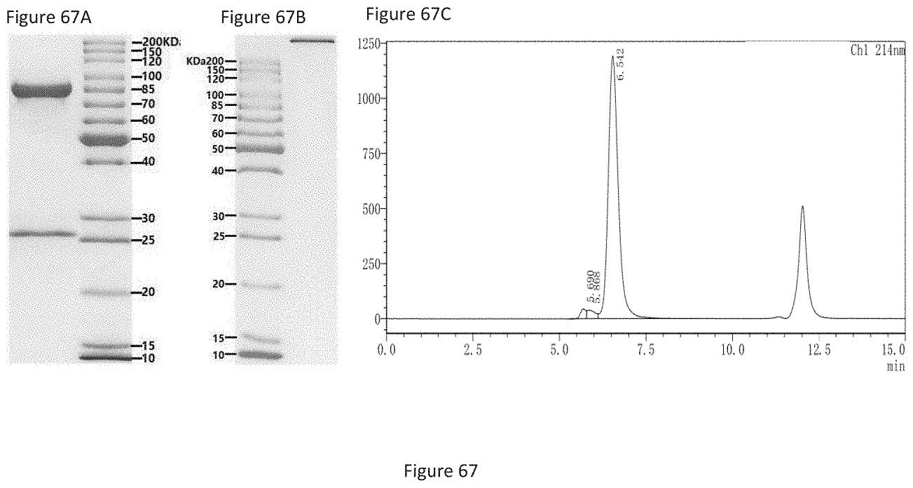

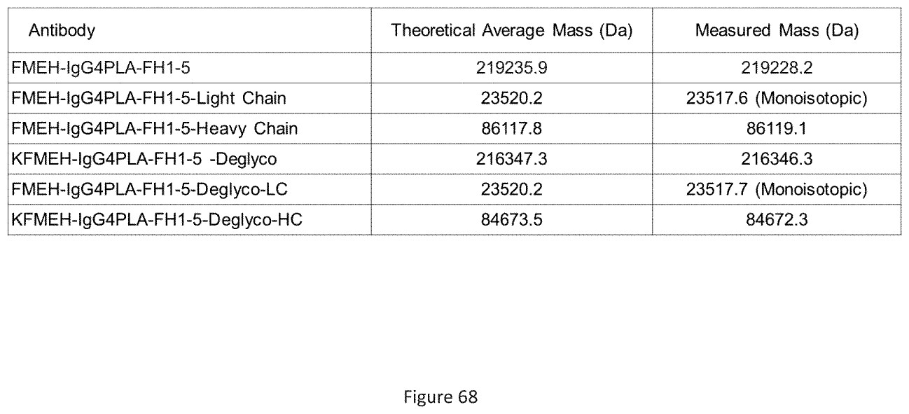

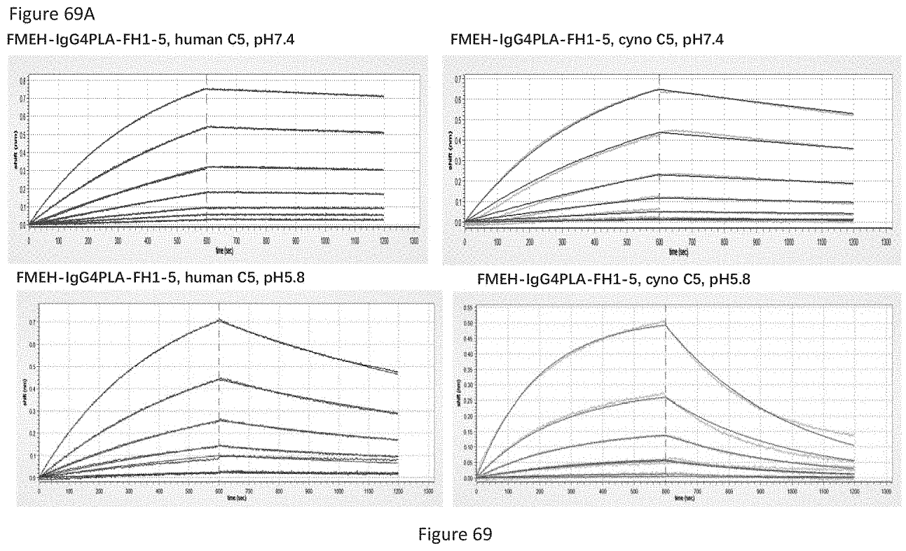

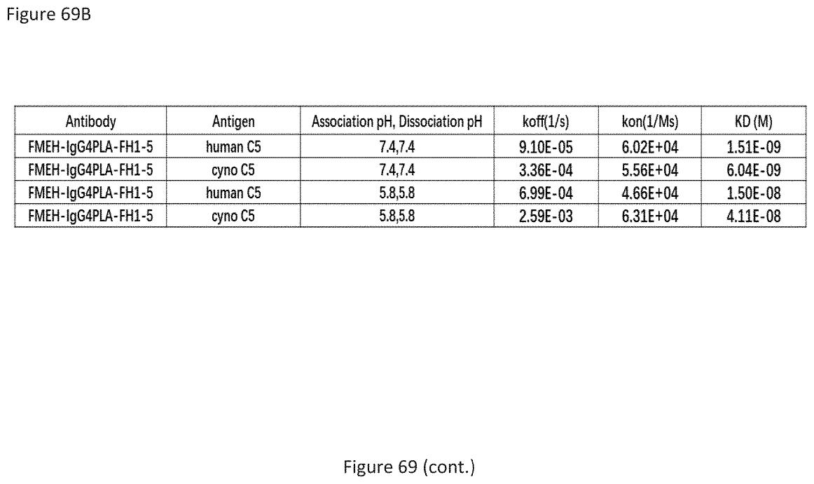

The mAb-FH fusion proteins still maintained the pH-dependent binding properties of the parent anti-C5 mAbs (FIG. 48 and FIG. 49), which is critical because such fusion proteins needs to behave as long lasting drugs, similar to the parent anti-C5 mAb (e.g., FMEH). The anti-C5 mAb-FH fusion protein was much more potent than their corresponding parent mAb (e.g., FMEH-IgG4PLA vs FMEH-IgG4PLA-FH SCR1-5) and benchmark regular anti-C5 mAbs (Eculizumab and Ravulizumab) in inhibiting rabbit RBC (FIG. 50) and PNH RBC lysis (FIG. 57). The anti-C5 mAb-fusion protein, but not the corresponding parent anti-mAb or benchmark anti-C5 mAbs (Eculizumab and Ravulizumab), was further shown to inhibit C3 opsonization of rabbit and PNH RBCS (FIG. 51 and FIG. 58).

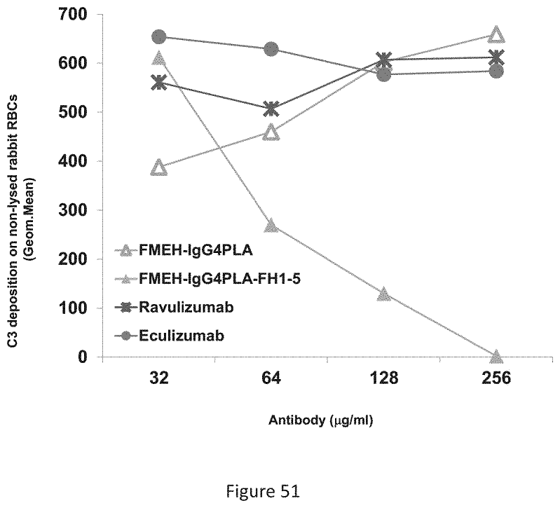

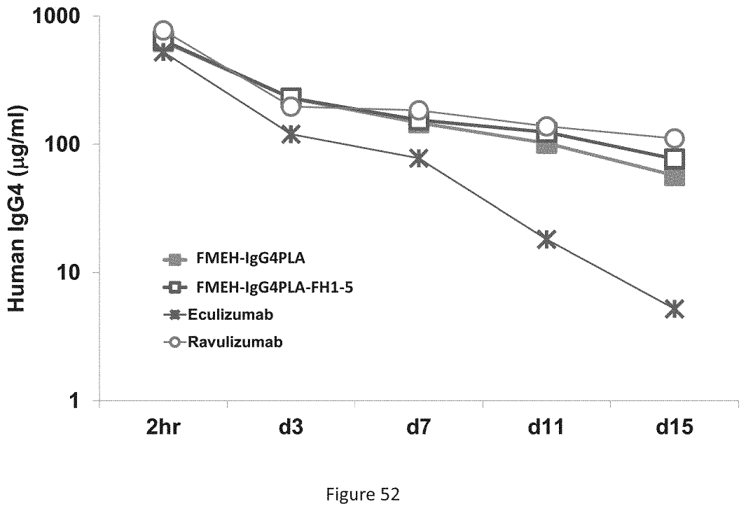

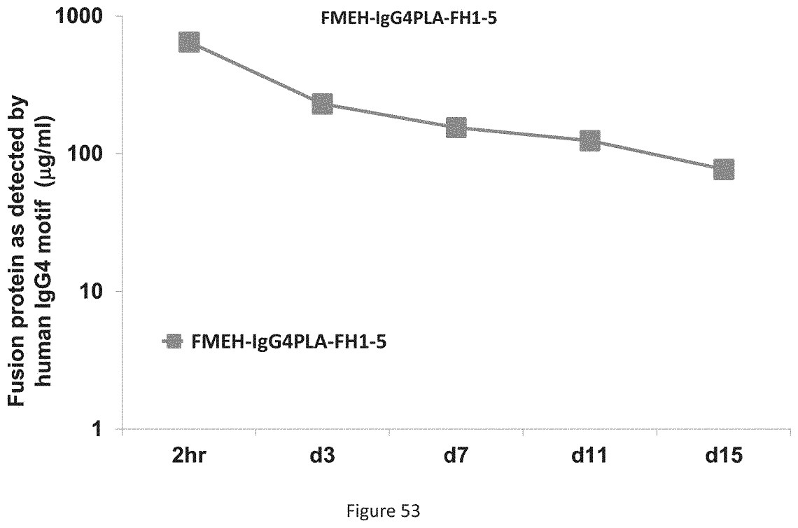

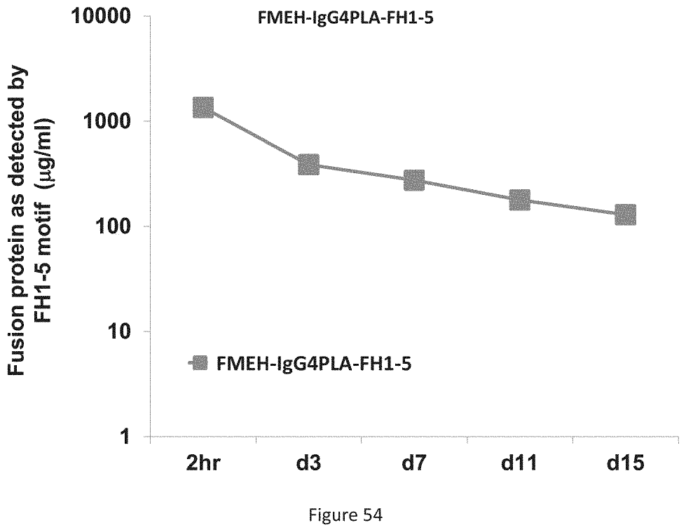

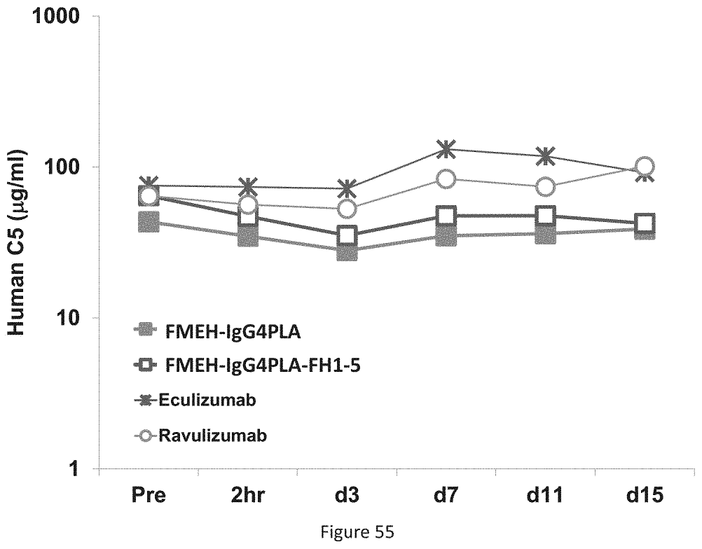

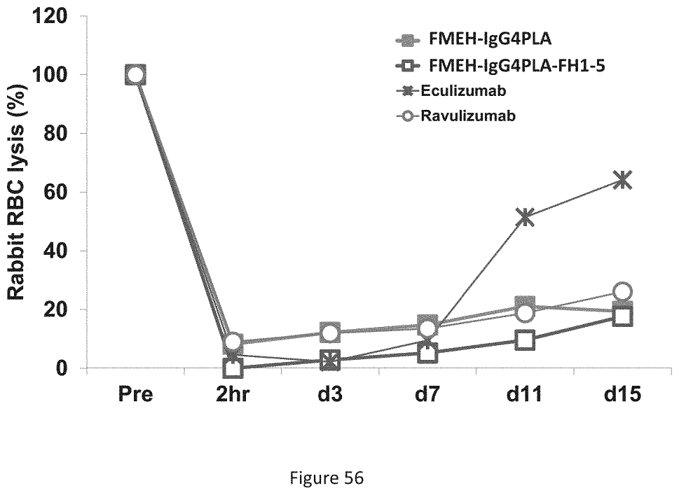

Moreover, anti-C5 mAb-FH fusion protein was demonstrated to have better PK (half-life) in C5 humanized mice than conventional benchmark anti-C5 mAb (Eculizumab) and similar PK to 2nd generation long lasting benchmark anti-C5 mAb (Ravulizumab), but better PD (pharmacodynamics) in rabbit RBC lysis test than both benchmark anti-C5 mAbs (due to its bi-functional property) (FIG. 52 through FIG. 56).

The materials and methods used in this Example are now described.

Sandwich ELISA for detection of human C5 in mice: Sandwich ELISA for detection of human C5 in NOD/SCID or FcRn/SCID mice expressing human C5 after hydrodynamic injection of human C5 cDNA plasmid: 96-well plates were coated with an anti-human C5 antibody (Quidel, A217) at a final concentration of 2 μg/mL in bicarbonate buffer at 37° C. for 1 hr. Following washes with PBS containing 0.05% Tween-20, the plates were incubated with diluted plasma samples in blocking solution at RT for 1 hr. After washing, the plates were incubated with biotinylated anti-human C5 mAb 9G6) in blocking solution at RT for 1 hr, washed again and incubated with avidin or streptavidin conjugated to horseradish peroxidase (BD pharmigen) in blocking solution at RT for 1 hr. After final washing, the plates were developed with HRP substrate for 3 min. The reaction was stopped with 2N H2504 and the plate was read at 450 nm in a micro plate reader. Sandwich ELISA for detection of human IgG4 in mice.

Sandwich ELISA for detection of human IgG4: Sandwich ELISA for detection of human IgG4 in mice treated with anti-human C5 IgG4 mAbs or anti-C5 mAb-FH SCR1-5 fusion proteins: 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with diluted plasma samples in blocking solution at RT for 1 hr. After washing, the plates were incubated with anti-human IgG4 HRP (1:2000 dilution, Invitrogen, A10654) in blocking solution at RT for 1 hr. After washing, the plates were developed with HRP substrate for 3 min. The reaction was stopped with 2N H2504 and the plate was read at 450 nm in a micro plate reader.

Sandwich ELISA for detection of human IgG4→FH1-5 fusion in mice treated with anti-human C5 IgG4→FH1-5 fusion: 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with blocking solution at 37° C. for 1 hr. After washing, the plates were incubated with diluted plasma samples in blocking solution at RT for 1 hr. After washing, the plates were incubated with biotin-conjugated anti-human FH (1:100 dilution, Thermo Scientific, MA5-17735) in blocking solution at RT for 1 hr. After washing, the plates were incubated with streptavidin-HRP (1:1000 dilution, BD Biosciences, 554066) in blocking solution at RT for 1 hr. After washing, the plates were developed with HRP substrate (Thermo Scientific, 34029) for 3 min. The reaction was stopped with 2N H₂SO₄ and the plate was read at 450 nm in a micro plate reader.

Sandwich ELISA for detection of intact FMEH-IgG4PLA-FH1-5 fusion protein in mouse plasma samples: 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with blocking solution at 37° C. for 1 hr. After washing, the plates were incubated with diluted plasma samples in blocking solution at RT for 1 hr. After washing, the plates were incubated with biotin-conjugated anti-human factor-H antibody (1:100 dilution, Thermo Scientific, MA5-17735) in blocking solution at RT for 1 hr. After washing, the plates were incubated with streptavidin-HRP (1:1000 dilution, BD Biosciences, 554066) in blocking solution at RT for 1 hr. After washing, the plates were developed with HRP substrate (Thermo Scientific, 34029) for 3 min. The reaction was stopped with 2N H₂SO₄ and the plate was read at 450 nm in a micro plate reader.

Sheep red blood cell lysis test: Sheep RBCs (1×10⁷ cells per assay sample prepared in PBS, Complement Technology Inc) were incubated at 37° C. for 20 min with 50% normal human serum (NHS, from Complement Technology Inc) in gelatin veronal buffer (GVB2+, Sigma; total assay volume: 100 μL). Before addition to the sheep RBCs, NHS was pre-incubated with anti-C5 mAbs for 1 hr at 4° C. Lysis reaction was stopped by addition of ice-cold 40 mM EDTA in PBS. The incubation mixtures were centrifuged for 5 min at 1500 rpm and the supernatant was collected and measured for OD405 nm. Samples without NHS or with EDTA added were used as negative lysis controls, and a sample of sheep RBCs lysed completely with distilled water was used as a positive control (100% lysis) against which % lysis in other samples was normalized.

Chicken red blood cell lysis assay: Chicken RBC (Rockland Immunochemicals Inc #R401-0050) were sensitized with anti Chiken Rabbit RBC antibody (Rockland Immunochemicals Inc #103-4139) (150 ug/mL) for 30 min and washed two times with GVB buffer. C5 humanized mouse lepirudin plasma pre-treated with mAb BB5.1 to block murine C5 activity and C5 depleted human serum (Quidel #A501) samples were diluted to 10% in GVB buffer (i.e., 5 mL to 50 mL final assay volume) and mixed with 5 mL of Antibody sensitized cRBC cells ul (5×10⁸/mL) in final volume of 50 mL and incubated at 37° C. for 30 min. Reaction was stopped with 100 mL of cold 10 mM EDTA in PBS. Cells were centrifuged at 1500 rpm for 5 min at 4° C. Collected supernatant was measured OD at 405.

Rabbit red blood cell lysis test: Rabbit RBCs (Rockland Immunochemicals Inc cat# R403-0100) (1×10⁷ cells per assay sample prepared in PBS, Complement Technology Inc) were incubated at 37° C. for 30 min with 25% normal human serum (NHS, from Complement Technology Inc) or 50% cynomolgus monkey serum (CMS, Innovative research) in gelatin veronal buffer (GVB2+EGTA, Sigma; total assay volume: 100 μL). Before addition to the rabbit RBCs, NHS or CMS was pre-incubated with anti-C5 mAbs or anti-C5 mAb-fH SCR1-5 fusion proteins for 1 hr at 4° C. Lysis reaction was stopped by addition of ice-cold 40 mM EDTA in PBS. The incubation mixtures were centrifuged for 5 min at 1500 rpm and the supernatant was collected and measured for OD405 nm. Samples without NHS or CMS or with EDTA added were used as negative lysis controls, and a sample of rabbit RBCs lysed completely with distilled water was used as a positive control (100% lysis) against which % lysis in other samples was normalized.

Rabbit red blood cell lysis test using hybrid human and mouse complement systems for pharmacodynamics study: To test the pharmacodynamics of various anti-hC5 mAbs and their fH SCR1-5 fusions in C5 humanized FcRn/SCID mice, rRBC lysis assay was done using C5-depleted human serum mixed with plasma of the testing C5 humanized FcRn/SCID mice (as a source of human C5). Mouse C5 in the assay mixture was blocked with 2 anti-mouse C5 antibodies BB5.1 and 21A9 (400 ug/mL each in final reaction). Mouse lepirudin plasma and C5-depleted human serum(Quidel #A501) samples were diluted to 10% in GVB EGTA buffer and mixed with 5 uL of rabbit RBC cells ul (5×10⁸/mL) in final volume of 50 uL and incubated at 37° C. for 30 min. Reaction was stopped with 100 uL of cold 10 mM EDTA in PBS. Cells were centrifuged at 1500 rpm for 5 min at 4° C. Collected supernatant was measured OD at 405.

Human C5 transposon plasmid construction: C5 cDNA from pCMV Sport6 sub cloned into pCAGGS vector at EcoRI site. At 5′ site of the enhancer at Hinc II site SB IR/DR(L) transposon recognition sequence and at 3′ site after rBG Stu I site SB IR/DR(R) recognition sequence was cloned by infusion cloning method. For hydrodynamic injection into NOD/SCD mice 2 μg of pCMV-T7-SB100 plasmid and 25 μg of hC5 in pCAGGS with transposon sites were injected by tail vain. Transfection efficiency was checked after day 1 by hC5 ELISA. Stable integration was again checked hC5 ELISA by 2 weeks post injection.

Histidine Scanning: Histidine Scanning of humanized mAb 2G1 (VH-11801 and VL-1901):HEK cells in 24 well plates and transfected with 1 μg of VH and 2 μg of VL plasmid using 8 μL of X-TremeGene HP DNA Transfection Reagent (Roche#6366546001). Supt was collected 2 days post transfection and used for Octet assay. pH dependent dissociation of 11801 histidine mutants were analyzed by Biolayer interferometry on an Octet Red E instrument (ForteBio Inc.). Anti-human IgG Biosensors (ForteBio#18-5060). Antibody was captured onto sensors by dipping them into 200 μL of transfection supernatant for 600 sec. Later these biosensors were incubated with hC5 for 600 Sec fallowed by dissociated at pH 7.4 and pH 5.8.

Parsimonious mutagenesis: To identify point amino acid substitution mutation that improves C5 binding at pH 7.4, all 6 complementary-determining regions (CDRs) of mAb H1-8/L1-9 was individually mutated to other 19 amino acids by a site-directed mutagenesis method. Oligonucleotides encoded designed mutation for each position are used to introduce mutations to the targeted CDR position by QuikChange® (Agilent) or Q5® Site-Directed Mutagenesis (NEB). To screen binding improved variant, 88 clones from each transfected reaction were picked and scFv secreted from the E. coli cell media is tested in a capture ELISA. In the capture ELISA, titrated amount of anti-Fd antibody is used to coat the wells to capture scFv from the bacteria supernatant. This was followed by incubation with biotinylated antigen. Binding signal is detected using HRP conjugated anti-Lc antibody followed by incubation with TMB substrate. The reaction is quenched with 0.2 M H2504, and the plates were read at 450 nm. Clones exhibiting an optical density (OD) 450 nm signal greater than the parental clone were picked. ScFv from regrown supernatant are re-assayed by ELISA (as above) in duplicate to confirm positive results. The binding improved clones are further confirmed in a direct binding ELISA where antigen is coated in the ELISA wells, and calculated amount of scFv determined by a scFv quantitative ELISA is used. Clones that repeatedly exhibited greater than parental scFv binding were sequenced.

Measurement of pH dependent binding/dissociation: pH dependent binding of affinity matured H1-8L1-9 anti-C5 mAb mutants and corresponding FH SCR 1-5 fusion proteins: 2-3×10⁶/mL ExpiCHO were seeded in to 24 well plates and transfected with 2 μg of VH and 4 μg of VL plasmid using 8 μL of X-TremeGene HP DNA Transfection Reagent (Roche#6366546001). Supt was collected 2 days post transfection and used for Octet assay. pH dependent dissociation of histidine mutants was analyzed by Biolayer interferometry on an Octet Red E instrument (ForteBio Inc.). Streptavidin Biosensors (ForteBio#18-5019) were coated with 3 μg/mL capture-select biotin anti human IgG 4 fab (Thermo fisher #7102902100) for 250-300 Sec and quenched with 2 mM Biocytin for 600 sec. These biosensors were incubated with 10 μg/mL hC5 (Complement tech#A320) for 600 Sec to saturate the non-specific binding of hC5 to biosensors. Antibody was captured onto sensors by dipping them into 200 μL of transfection supernatant for 600 sec. Later these biosensors were incubated with hC5 for 600 sec followed by Dissociated at pH 7.4 and pH 5.8.

Large-scale transfection: Large-scale ExpiCHO transfection protocol: For large-scale production of mAbs and mAb-FH fusion proteins for in vivo study, ExpiCHO transfection system (Gibco#A29133) was used according to manufactures instructions using max-titer protocol. For 200 mL of Expi CHO medium 150 μg VH and 300 μg VL used for transfection.

Affinity analysis of FMEH-IgG4PLA and FMEH-IgG4PLA-FH1-5 to human or Cynomolgus FcRn: Surface Plasmon resonance analysis was used to measure the association and dissociation rate constant for binding of FMEH-IgG4PLA and FMEH-IgG4PLA-FH1-5 to immobilized human or Cyno FcRn, using BIAcore 3000 instrument (Biacore AB, Uppsala, Sweden) and all Biacore experiments were performed at 25° C. The carboxylated dextran matrix of a CM5 sensor chip was used to couple the purified human or Cyno FcRn, by amine coupling chemistry to obtain 280 RU or 200 RU surface density, respectively. The FMEH-IgG4PLA or FMHE-IgG4PLA-FH1-5 was diluted to 200, 100, 50, 25, 12.5, and 0 nM in HBS (HEPES buffer saline) buffer and the samples were injected for 200 s and dissociation of bound analyte was allowed to proceed for 300 s. The data were analyzed by the BIA evaluation software 3.2 assuming bivalent binding model.

Determination of pharmacokinetics in C5-humanized mice of intact FMEH-IgG4PLA-FH1-5 fusion protein: C5 and FcRn humanized SCID mice were injected with 40 mg/kg dose of FMEH-IgG4PLA-FH1-5 fusion protein via retro-orbital route. EDTA plasma was collected at various time points after drug administration as indicated in data graphs.

Characterization of FMEH-IgG4PLA-FH1-5 by SDS-PAGE: SDS-PAGE (sodium dodecyl sulfate-polyacrylamide gel electrophoresis) was used to characterize FMEH-IgG4PLA-FH1-5 protein. FMEH-IgG4PLA-FH1-5 is in PBS buffer with or without reducing agent. Samples were heated at 99° C. for 10 min and 5 ug of the protein marker and 2 ug of the reducing and non-reducing samples were loaded into 12% SDS-PAGE gels. The gels were run at 80 v for 30 min and then run at 170 v for 1 h, followed by staining the gels in the Coomasie Brilliant Blue-R-250 solution. Gel was de-stained and imaged using the Gel Doc XR Imaging system (Bio-Rad Laboratories, Inc., USA).

Purity analysis of FMEH-IgG4PLA-FH1-5 by SEC-HPLC: SEC-HPLC (Size exclusion chromatography-high-performance liquid chromatography) was used to measure purity of FMEH-IgG4PLA-FH1-5 protein. LC-20AT HPLC system (Shimadzu Scientific Instruments, Japan) and a TSK gel G3000SWXL column (Tosoh Corporation, Japan) were used in the experiment. SEC-HPLC experiments were performed under the running conditions of oven temperature of 35° C., flow rate 1 mL/min and mobile phase PBS (Phosphate-buffered saline). A TSK gel G3000SWXL column was connected to the LC-20AT HPLC system and equilibrated with PBS. 0.242 mg of FMEH-IgG4PLA-FH1-5 protein solution was injected and the absorbance at 214 nm was detected for 15 min. The data were analyzed by the Lab Solutions software.

Binding affinity analysis of FMEH-IgG4PLA-FH1-5 to human and Cynomolgus C5: Bio-Layer Interferometry was used to measure binding association and dissociation rate constant for FMEH-IgG4PLA and FMEH-IgG4PLA-FH1-5 to soluble human and Cynomolgus C5. Bio-Layer Interferometry Gator (Probe Life Inc., China) was used and all experiments were performed at 30° C. The HFC (Anti-HIgG Fc) Probes were dipped in K Buffer (Phosphate-buffered saline, containing Bovine serum albumin) for 300 sec. The HFC Probes were then dipped in FMEH-IgG4PLA-FH1-5 to obtain 1 nm surface density. After a 200-sec baseline dip in K Buffer, the HFC Probes were dipped in solutions containing purified human or Cynomolgus C5 at varying concentrations (40, 20, 10, 5, 2.5, 1.25 and 0.625 nM) for 600 sec, followed by a 600 sec dissociation period in K Buffer. The experiment was performed under either pH 7.4 or pH 5.8 buffer condition to determine the effect of pH on the binding kinetics. The data were analyzed by the Gator evaluation software (Probe Life Inc., China) using 1:1 binding model.

Molecular weight analysis of FMEH-IgG4PLA-FH1-5: Mass spectrometry was used to measure molecular weight of FMEH-IgG4PLA-FH1-5. U3000 UPLC system and Q Exactive Plus Mass Spectrometer (Thermo Fisher Scientific Inc., USA) was used in the experiments. De-glycosylated FMEH-IgG4PLA-FH1-5 was prepared using Peptide-N-Glycosidase F and reduced FMEH-IgG4PLA-FH1-5 was prepared using dithiothreitol. Intact, de-glycosylated and reduced FMEH-IgG4PLA-FH1-5 were separated and detected by LC-MASS, respectively, as described by the manufacturer. The data were analyzed by the Biopharma Finder software (Thermo Fisher Scientific Inc., USA).

PNH patient RBC lysis assay: PNH RBCs were suspend in AP buffer (GVB+5 mM Mg²⁺+20 mM EGTA, pH 6.4), and then incubated at 37° C. for 40 min with 0.2 M HCl acidified normal human serum in gelatin veronal buffer (Complement Technology, Texas USA). NHS was pre-incubated with FMEH-IgG4PLA-FH1-5, Eculizumab or Ravulizumab for 1 hr at 4° C. HCl-treated (0.2 M) PNH RBCs and pre-incubated antibody serum was then mixed and incubated xxx min. in a tube. Total reaction volume is 50 ul. The lysis reaction was stopped by addition ice-cold 40 mM EDTA in PBS. The incubation mixtures were centrifuged for 5 min at 1500 rpm and the supernatant was collected and measured for OD405 nm. A sample with EDTA added was used as a negative lysis control, and a sample of PNH RBCs lysed completely with distilled water was used as a positive control (100% lysis). % of lysis was calculated using the formula (OD405_{(specific ab con.)}−OD405_{(40 mM EDTA)})/((OD405_{(water)}−OD405_{(40 mM EDTA)})*100%.

C3b deposition analysis: Un-lysed RBCs following the RBC lysis assay were collected by centrifugation. Cells were washed with PBS and then resuspended in PBS buffer. APC-labeled anti-C3b antibody (Biolegend, California USA) was added and incubated on ice for 30 min in dark. C3b+ staining cells were gated and analyzed using flow cytometer CytoFLEX (Beckman Coulter, Suzhou, China) or FACSCelesta (BD Biosciences, California USA). ND means not detectable.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A fusion protein comprising an antibody that specifically binds to human C5 and a fusion protein partner.

2. The fusion protein of claim 1, wherein the binding of the antibody is pH-dependent, wherein the antibody binds more strongly to C5 at a neutral pH than it does at an acidic pH.

3. The fusion protein of claim 1, wherein the antibody is a bi-functional antibody fusion protein.

4. The fusion protein of claim 3, wherein the antibody fusion protein inhibits C3 activation.

5. The fusion protein of claim 1,
wherein the fusion protein comprises a complement control protein.

6. The fusion protein of claim 1, wherein the fusion protein partner comprises at least one selected from the group consisting of a complement receptor 1 (CR1) or a fragment thereof, a membrane cofactor protein (MCP) or a factor thereof, a C4b-binding protein (C4BP) or a fragment thereof, a decay-accelerating factor (DAF) or a fragment thereof, an Apolipoprotein E (ApoE) or a fragment thereof, a Factor H (FH) protein or a fragment thereof, a human IgG4 or a fragment thereof, a linker, and any combination thereof.

7. The fusion of claim 6, wherein the fragment of FH comprises short consensus repeat (SCR) domains 1-5 of the FH protein.

8. The fusion protein of claim 1, wherein the antibody is selected from the group consisting of:
a) an antibody comprising the CDRs: VH-CDR1: SEQ ID NO:3; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:11, or a variant or variants thereof;
b) an antibody comprising the CDRs: VH-CDR1: SEQ ID NO:3; VH-CDR2: SEQ ID NO:4; VH-CDR3: SEQ ID NO:5; VL-CDR1: SEQ ID NO:14; VL-CDR2: SEQ ID NO:9; and VL-CDR3: SEQ ID NO:10, or a variant or variants thereof;
c) an antibody comprising the CDRs: VH-CDR1: SEQ ID NO:17; VH-CDR2: SEQ ID NO:4; VH-CDR3: SEQ ID NO:5; VL-CDR1: SEQ ID NO:8; VL-CDR2: SEQ ID NO:9; and VL-CDR3: SEQ ID NO:10, or a variant or variants thereof;
d) an antibody comprising the CDRs: VH-CDR1: SEQ ID NO:20; VH-CDR2: SEQ ID NO:4; VH-CDR3: SEQ ID NO:5; VL-CDR1: SEQ ID NO:23; VL-CDR2: SEQ ID NO:9; and VL-CDR3: SEQ ID NO:10, or a variant or variants thereof;
e) an antibody comprising the CDRs: VH-CDR1: SEQ ID NO:3; VH-CDR2: SEQ ID NO:26; VH-CDR3: SEQ ID NO:5; VL-CDR1: SEQ ID NO:8; VL-CDR2: SEQ ID NO:9; and VL-CDR3: SEQ ID NO:29, or a variant or variants thereof;
f) an antibody comprising the CDRs: VH-CDR1: SEQ ID NO:3; VH-CDR2: SEQ ID NO:34; VH-CDR3: SEQ ID NO:5; VL-CDR1: SEQ ID NO:8; VL-CDR2: SEQ ID NO:9; and VL-CDR3: SEQ ID NO:10, or a variant or variants thereof;
g) an antibody comprising the CDRs: VH-CDR1: SEQ ID NO:37; VH-CDR2: SEQ ID NO:38; VH-CDR3: SEQ ID NO:39; VL-CDR1: SEQ ID NO:23; VL-CDR2: SEQ ID NO:9; and VL-CDR3: SEQ ID NO:10, or a variant or variants thereof;
h) an antibody comprising the CDRs: VH-CDR1: SEQ ID NO:42; VH-CDR2: SEQ ID NO:43; VH-CDR3: SEQ ID NO:44; VL-CDR1: SEQ ID NO:23; VL-CDR2: SEQ ID NO:9; and VL-CDR3: SEQ ID NO:10, or a variant or variants thereof;
i) an antibody comprising the CDRs: VH-CDR1: SEQ ID NO:47; VH-CDR2: SEQ ID NO:48; VH-CDR3: SEQ ID NO:49; VL-CDR1: SEQ ID NO:23; VL-CDR2: SEQ ID NO:9; and VL-CDR3: SEQ ID NO:10, or a variant or variants thereof;
j) an antibody comprising the CDRs: VH-CDR1: SEQ ID NO:52, VH-CDR2: SEQ ID NO:53, VH-CDR3: SEQ ID NO:54, VL-CDR1: SEQ ID NO:23, VL-CDR2: SEQ ID NO:9, and VL-CDR3: SEQ ID NO:10, or a variant or variants thereof;
k) an antibody comprising the CDRs: VH-CDR1: SEQ ID NO:47, VH-CDR2: SEQ ID NO:57, VH-CDR3: SEQ ID NO:49, VL-CDR1: SEQ ID NO:23, VL-CDR2: SEQ ID NO:9, and VL-CDR3: SEQ ID NO:10, or a variant or variants thereof;
l) an antibody comprising the CDRs: VH-CDR1: SEQ ID NO:37, VH-CDR2: SEQ ID NO:62, VH-CDR3: SEQ ID NO:39, VL-CDR1: SEQ ID NO:23, VL-CDR2: SEQ ID NO:9, and VL-CDR3: SEQ ID NO:10, or a variant or variants thereof;
m) an antibody comprising the CDRs: VH-CDR1: SEQ ID NO:42, VH-CDR2: SEQ ID NO:65, VH-CDR3: SEQ ID NO:44, VL-CDR1: SEQ ID NO:23, VL-CDR2: SEQ ID NO:9, and VL-CDR3: SEQ ID NO:10, or a variant or variants thereof; and
n) an antibody comprising the CDRs: VH-CDR1: SEQ ID NO:52, VH-CDR2: SEQ ID NO:68, VH-CDR3: SEQ ID NO:54, VL-CDR1: SEQ ID NO:23, VL-CDR2: SEQ ID NO:9, and VL-CDR3: SEQ ID NO:10, or a variant or variants thereof.

9. The fusion protein of claim 1, wherein the antibody comprises a heavy chain comprising an amino acid sequence selected from the group consisting of SEQ ID NO:2, or a variant thereof; SEQ ID NO:19, or a variant thereof; SEQ ID NO:22, or a variant thereof; SEQ ID NO:28, or a variant thereof; SEQ ID NO:36, or a variant thereof; SEQ ID NO:41, or a variant thereof; SEQ ID NO:46, or a variant thereof; SEQ ID NO:51, or a variant thereof; SEQ ID NO:56, or a variant thereof; SEQ ID NO:59, or a variant thereof; SEQ ID NO:72, or a variant thereof; SEQ ID NO:76, or a variant thereof; SEQ ID NO:78, or a variant thereof and SEQ ID NO:80, or a variant thereof.

10. The fusion protein of claim 1, wherein the antibody comprises a light chain comprising an amino acid sequence selected from the group consisting of SEQ ID NO:7, or a variant thereof; SEQ ID NO:13, or a variant thereof; SEQ ID NO: 16, or a variant thereof; SEQ ID NO:25, or a variant thereof; SEQ ID NO:31, or a variant thereof and SEQ ID NO:74, or a variant thereof.

11. The fusion protein of claim 1, wherein the antibody is selected from the group consisting of:
a) an antibody comprising a heavy chain comprising the amino acid sequence of SEQ ID NO:2 and a light chain comprising the amino acid sequence of SEQ ID NO:13, or a variant or variants thereof;
b) an antibody comprising a heavy chain comprising the amino acid sequence of SEQ ID NO:2 and a light chain comprising the amino acid sequence of SEQ ID NO:16, or a variant or variants thereof;
c) an antibody comprising a heavy chain comprising the amino acid sequence of SEQ ID NO:19 and a light chain comprising the amino acid sequence of SEQ ID NO:7, or a variant or variants thereof;
d) an antibody comprising a heavy chain comprising the amino acid sequence of SEQ ID NO:22 and a light chain comprising the amino acid sequence of SEQ ID NO:25, or a variant or variants thereof;
e) an antibody comprising a heavy chain comprising the amino acid sequence of SEQ ID NO:28 and a light chain comprising the amino acid sequence of SEQ ID NO:31, or a variant or variants thereof;
f) an antibody comprising a heavy chain comprising the amino acid sequence of SEQ ID NO:36 and a light chain comprising the amino acid sequence of SEQ ID NO:7, or a variant or variants thereof;
g) an antibody comprising a heavy chain comprising the amino acid sequence of SEQ ID NO:41 and a light chain comprising the amino acid sequence of SEQ ID NO:25, or a variant or variants thereof;
h) an antibody comprising a heavy chain comprising the amino acid sequence of SEQ ID NO:46 and a light chain comprising the amino acid sequence of SEQ ID NO:25, or a variant or variants thereof;
i) an antibody comprising a heavy chain comprising the amino acid sequence of SEQ ID NO:51 and a light chain comprising the amino acid sequence of SEQ ID NO:25, or a variant or variants thereof;
j) an antibody comprising a heavy chain comprising the amino acid sequence of SEQ ID NO:56 and a light chain comprising the amino acid sequence of SEQ ID NO:25, or a variant or variants thereof;
k) an antibody comprising a heavy chain comprising the amino acid sequence of SEQ ID NO:59 and a light chain comprising the amino acid sequence of SEQ ID NO:25, or a variant or variants thereof;
l) an antibody comprising a heavy chain comprising the amino acid sequence of SEQ ID NO:72 and a light chain comprising the amino acid sequence of SEQ ID NO:74, or a variant or variants thereof;
m) an antibody comprising a heavy chain comprising the amino acid sequence of SEQ ID NO:76 and a light chain comprising the amino acid sequence of SEQ ID NO:74, or a variant or variants thereof;
n) an antibody comprising a heavy chain comprising the amino acid sequence of SEQ ID NO:78 and a light chain comprising the amino acid sequence of SEQ ID NO:74, or a variant or variants thereof; and
o) an antibody comprising a heavy chain comprising the amino acid sequence of SEQ ID NO:80 and a light chain comprising the amino acid sequence of SEQ ID NO:74, or a variant or variants thereof.

12.-57. (canceled)

58. The fusion protein of claim 8, wherein the antibody comprises at least one substitution selected from the group consisting of a substitution at proline 4 (P4) in VH CDR2, relative to SEQ ID NO:4, a substitution at threonine 9 in VH CDR2, relative to SEQ ID NO:4, a substitution at valine 16 (V16) in VH CDR3, relative to SEQ ID NO:5, a substitution at leucine 9 (L9) in VH CDR1, relative to SEQ ID NO:20, and a substitution at leucine 9 (L9) in VH CDR1, relative to SEQ ID NO:20.

59. The fusion protein of claim 58, wherein the substitution at proline 4 (P4) is one substitution selected from the group consisting of P4→F4 (P4F), P4→L4 (P4L), P4→M4 (P4M), P4→W4 (P4W), and P4→I4 (P4I).

60. The fusion protein of claim 58, wherein the antibody comprises a substitution at proline 4 (P4) in VH CDR2, relative to SEQ ID NO:4, and a substitution at threonine 9 in VH CDR2, relative to SEQ ID NO:4.

61. The fusion protein of claim 60, wherein the substitution at proline 4 (P4) is one substitution selected from the group consisting of P4→F4 (P4F), P4→L4 (P4L), P4→M4 (P4M), P4→W4 (P4W), and P4→I4 (P4I); and the substitution at threonine 9 (T9) is one substitution selected from the group consisting of T9→H9 (T9H), T9→F9 (T9F), T9→L9 (T9L), T9→M9 (T9M), T9→W9 (T9W), and T9→I9 (T9I).

62. (canceled)

63. The fusion protein of claim 58, wherein the substitution at valine 16 (V16) is one substitution selected from the group consisting of V16→F16 (V16F), V16→E16 (V16E) and V16→W16 (V16W).

64. (canceled)

65. The fusion protein of claim 58, wherein the substitution at leucine 9 (L9) is one substitution selected from the group consisting of L9→W9 (L9W), L9→I9 (L9I), L9→V9 (L9V), L9→Y9 (L9Y), and L9→F9 (L9F).

66. The fusion protein of claim 8, wherein the antibody comprises a substitution of two or more substitutions selected from the group consisting of proline 4 (P4) in VH CDR2, relative to SEQ ID NO:4, threonine 9 (T9) in VH CDR2, relative to SEQ ID NO:4, valine 16 (V16) in VH CDR3, relative to SEQ ID NO:5, and leucine 9 (L9) in VH CDR1, relative to SEQ ID NO:20.

67. The fusion protein of claim 66, wherein the antibody comprises two or more substitutions selected from the group consisting of L9I/P4M, L9I/P4W, L9I/P4F, L9F/P4M, L9F/P4W, L9F/P4F, L9I/P4M/V16W, L9I/P4W/V16W, L9I/P4F/V16W, L9F/P4M/V16W, L9F/P4W/V16W, L9F/P4F/V16W, L9I/P4W/V16E, L9I/P4W/V16E, L9I/P4F/V16E, L9F/P4M/V16E, L9F/P4W/V16E, L9F/P4F/V16E, L9I/P4M/T9H/V16W, L9I/P4W/T9H/V16W, L9I/P4F/T9H/V16W, L9F/P4M/T9H/V16W, L9F/P4W/T9H/V16W, L9F/P4F/T9H/V16W, L9I/P4M/T9H/V16E, L9I/P4W/T9H/V16E, L9I/P4F/T9H/V16E, L9F/P4M/T9H/V16E, L9F/P4W/T9H/V16E, and L9F/P4F/T9H/V16E.

68. A method of treating a complement pathway-mediated disease or disorder in an individual, comprising the step of administering to said individual the fusion protein of claim 1.

69. The method of claim 68, wherein the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, myasthenia gravis, neuromyelitis optica, (NMO), multiple sclerosis, delayed graft function, antibody-mediated rejection, atypical hemolytic uremic (aHUS) syndrome, central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, or any combinations thereof.

70. A method of reducing the activity of a complement system of an individual, wherein the method comprises administering an antibody to the individual via a route of administration selected from the group consisting of enteral administration, parenteral administration, and a combination thereof, and wherein the antibody is the fusion protein of claim 1.

71. The method of claim 70, wherein the antibody is an antibody fragment selected from the group consisting of a Fab, Fab′, F(ab)2, F(ab′)2, scFv, and combinations thereof.

72. A cell comprising the fusion protein of claim 1.

73. The cell of claim 72, wherein the cell produces the fusion protein.

74. The cell of claim 73, wherein the cell is a hybridoma.

75. A genetically modified non-human animal that expresses human C5.

76. The genetically modified non-human animal of claim 75, wherein the non-human animal is a rodent.

77. The genetically modified non-human animal of claim 76, wherein the non-human animal is a mouse.

78. The genetically modified non-human animal of claim 76, wherein the non-human animal is a NOD/SCID mouse.

79. The genetically modified non-human animal of claim 76, wherein the non-human animal is a FcRn/SCID mouse.

80. A fusion protein comprising an anti-C5 antibody moiety and an FH or a functional fragment thereof.

81. The protein of claim 80, wherein the anti-C5 moiety comprises at least one histidine substitution.

82. The protein of claim 80, wherein the anti-C5 moiety comprises an IgG4 chain.

83. The protein of claim 82, wherein the IgG4 chain comprises a PLA mutation.

84. The protein of claim 80, wherein the FH or functional fragment thereof comprises domains 1-5 of the FH protein.
